(19) United States
(12) Patent Application Publication
(10) Pub. No.: US 2003/0134272 A1
(43) Pub. Date: Jul. 17, 2003

(54) Mutation analysis of the NF1 gene

(76) Inventors: Ludwine Messiaen, Gent (BE); Tom Callens, Kortruk (BE)

Correspondence Address: TRASK BRITT, P.O. BOX 2550, SALT LAKE CITY, UT, 84110, US

(21) Appl. No.: 10/128,560

(22) Filed: Apr. 18, 2002

Related U.S. Application Data
(63) Continuation of application No. PCT/EP00/10255, filed on Oct. 18, 2000.

(30) Foreign Application Priority Data
Oct. 18, 1999 (EP) 99870216.1
Jun. 5, 2000 (EP) 00870122.9

(51) Int. Cl. C12Q 1/70; C12Q 1/68

(52) U.S. Cl. 435/5; 435/6

(57) ABSTRACT

A method for mutation analysis of the neurofibromatosis 1 (NF1) gene of a patient includes extracting DNA from peripheral blood lymphocytes of the patient, establishing an EBV transformed B-lymphoblastoid cell line using lymphocytes from the patient, treating the EBV transformed B-lymphoblastoid cell line culture with puromycin, extracting RNA from cultures of the cell line immediately, amplifying the RNA using suitable primers, and obtaining peptide fragments by means of in vitro transcription/translation of the amplified fragments. The invention also relates to the identification of new hotspots and specific NF1 mutations. The invention also includes diagnostic kits for the detection of described specific mutations and hotspot domains, compounds correcting the structure of specific mutated NF1 proteins and in vitro and in vivo systems that may be used to screen for these therapeutic compounds.

### CROSS-REFERENCE TO RELATED APPLICATIONS

This application claims priority to International Application Number PCT/EP00/10255 filed on Oct. 18, 2000, designating the United States of America, International Publication Number WO 01/29251 (Apr. 26, 2001), the contents of which are incorporated herein by reference. Which International Application claims priority to European Patent Application No. 99870216.1 filed Oct. 18, 1999, the contents of which are incorporated herein by reference.

### BACKGROUND OF THE INVENTION

1. Technical Field

The present invention relates to the field of methods for genetic diagnosis of Neurofibromatosis type 1 (NF1). More particularly, the present invention relates to an optimized mutation analysis of the NF1 gene by a faster and more reliable protein truncation analysis leading to the identification of at least 83% of mutations in familial as well as sporadic NF1 patients fulfilling the N.I.H. diagnostic criteria. The current technology allows one to define the mutation profile of the NF1 gene.

2. State of the Art

Neurofibromatosis type 1 is one of the most common autosomal dominant disorders, affecting about 1:3500 individuals in all ethnic groups. The main characteristics are cutaneous and subcutaneous neurofibromas, café-au-lait (CAL) skin spots, iris Lisch nodules and freckling (Huson et al., 1994). Other features found in only a minority of patients include scoliosis, macrocephaly, pseudarthrosis, short stature, malignancies and learning disabilities. One of the most feared complications of NF1 is malignancy. The disorder shares some features common to heritable cancer syndromes due to mutations in a tumor suppressor gene. These are: i) a tissue-restricted occurrence of primary cancers in neural crest and myeloid lineage cells; ii) a decreased age of onset of malignancies compared to the general population; iii) the occurrence of multiple primary tumors in some patients.

The NF1 gene has been mapped to 17q11.2 and was positional cloned (Cawthon et al, 1990; Viskochil et al., 1990; Wallace et al., 1990). The NF1 gene is approximately 350 kb in size, contains 60 exons and codes for an ubiquitously expressed 11- to 13-kb transcript with an open reading frame coding for 2818 amino acids (Marchuk et al. 1991). The central portion of the coding sequence exhibits homology to the GTPase activating proteins (GAPs) and the protein can down regulate the Ras pathway through this GAP-related domain (reviewed by Kim and Tamanoi, 1998).

The mutation rate in the NF1 gene is one of the highest known for human genes (reviewed by Huson and Hughes, 1994) with approximately 50% of all NF1 patients presenting as sporadic cases expected to carry de novo germline mutations. For these patients, only identification of the pathogenic germline mutation allows for presymptomatic/prenatal testing in offspring. Mutation is located in one of both alleles resulting in the synthesis of a mutated form in addition to the WT protein. A decrease in the WT NF1 protein content results in a diseased state of the cell. Since NF1 is a dominant disorder, individuals that are heterozygous for a NF1 mutation may still express NF1 at 50% or reduced levels. In tumors, a second hit (loss of Heterozygosity or point mutation) in the remaining allele further reduces the neurofibromin content in the cells. Despite the high frequency of this disorder in all populations, relatively few mutations have been identified at the molecular level. Mutational analysis in NF1 patients has proven to be laborious and is hampered by the large size of the gene, the large number of exons, the presence of several pseudogenes (Cumming et al., 1993; Hulsebos et al., 1996) and the wide variety of mutations (i.e. nonsense, frameshift and missense mutations, small insertions or deletions, large deletions encompassing the total gene, translocations, etc). Many techniques have been applied for the study of NF1 mutations with detection rates varying from 10 to 65% (Abernathy et al, 1997, Fahsold et al., 2000, Ars et al., 2000).

A limited number of mutational “hotspots” have been reported: R1947X (C5839T) in exon 31, the 4-bp region between nucleotides 6789 and 6792 in exon 37, both implicated in about 2% of the NF1 patients (reviewed by Upadhyaya and Cooper, 1998). Also exon 4b is considered to contain mutational hotspots by others (Toliat et al., 2000). Recently we found that another mutational hotspot resides in exon 10b and harbors a missense mutation associated with aberrant splicing (Messiaen et al., 1999).

So far, no studies attempted to delineate the mutational spectrum in the NF1 gene by extensive analysis using a combined cascade of complementary techniques. A high mutation detection rate is especially important if genetic testing is requested for the offspring of sporadic patients in which only identification of the pathogenic mutation can allow for prenatal/predictive testing. Moreover, once a technology will be available that is able to find the mutation in the NF1 gene with high sensitivity; this will enable to help with the diagnosis of patients that do not fulfill to the N.I.H. diagnostic criteria yet.

The protein truncation test (PTT) is a form of mutation detection first described in 1993 by Roest et al. The PTT allows one to analyze the total coding region of a gene by in vitro transcription and translation of RT-PCR fragments and will specifically detect mutations that result in a truncated protein due to the occurrence of a premature stop codon or due to f.i. an in frame skipping of exons or segments of exons.

The protein truncation test (PTT) based on in vitro protein synthesis, was first applied by Heim et al. (1995) to the whole NF1 coding sequence in 21 unrelated patients and 14 mutations were identified at the cDNA level (66% detection rate). However, the authors were able to identify the mutation at the genomic level in only 11 of the patients, what brings us to a detection level at the genomic level of only 11/21 (52%). No reasons for this failure were discussed in the paper. Park et al. (1998) studied 14 unrelated patients and 10 mutations were disclosed (71%). Here too, one of the alterations seen in the cDNA could not be resolved at the genomic DNA level, what brings us to a detection level of 9/14 (64%). Whereas the experiments of Heim et al. (1995) started from either blood cells or lymphoblastoid cell lines, Park et al. (1998) exclusively used peripheral blood.

In order to be able to implement high-throughput screening using PTT, major improvements of the procedures are required (Den Dunnen and Van Ommen, 1999). Especially, laboratory testing for NF1 mutations is difficult. A protein truncation test is commercially available, but its sensitivity, specificity and predictive value have not been established (Rasmussen and Friedman, 2000).

### BRIEF SUMMARY OF THE INVENTION

The present invention provides an optimized mutation analysis method for the NF1 gene which is faster and more reliable than presently known protein truncation analysis systems. This optimized system can be applied to develop a kit that can be used for fast genetic NF1 diagnosis.

In addition, the invention also relates to the use of this optimized method to characterize new hotspot domains and specific mutations allowing the definition of the mutation profile of the NF1 gene.

The present invention relates more particularly to a method for mutation analysis of the NF1 gene of a patient comprising the steps of (see FIG. 1 and 2):

a) isolating peripheral blood lymphocytes of said patient,

b) establishing an EBV transformed B-lymphoblastoid cell line with said peripheral blood lymphocytes of said patient, or short-term culturing of the blood lymphocytes by phytohaemaglutinin (PHA) stimulation

c) treatment of the EBV transformed B-lymphoblastoid cell line or the short term cultures with a protein synthesis inhibitor,

d) immediate extraction of RNA of cultures of said EBV transformed B-lymphoblastoid cell line,

e) amplifying said RNA using suitable primers generating cDNA fragments covering the whole NF1 gene coding region (RT-PCR), and

f) obtaining peptide fragments by means of in vitro transcription/translation of said amplified fragments of step e).

According to a further embodiment, the present invention also relates to a method as described above wherein f) is followed by at least on of the following steps (see FIG. 1):

g) separation of said peptide fragments;

h) cycle sequencing of the cDNA fragments that resulted in a truncated peptide in the PTT assay. Cycle sequencing of cDNA prepared from EBV transformed B-lymphoblastoid cell lines that were treated with and without a protein synthesis inhibitor are compared. This allows, as explained below, to detect more easily the aberrant fragments and allows to give information on the stability of the mutant transcript in the affected cells.

i) evaluation of the cycle sequencing chromatograms with respect to putative splice sites using “Splice Site Prediction using Neural Networks” (http://www-hgc.lbl.gov/projects/splice.html, Messiaen et al. (2000)) if internal cDNA deletions are seen to allow to determine the plausible site of the genomic alteration.

j) Identification of the genomic mutation by cycle sequencing of the exons of interest.

Genetic analysis creates the possibility of finding gene mutations at an early stage in the disease development so symptoms can be reduced or even stopped in its early phase, if treatment becomes available. The present invention provides a method for the genetic analysis of the neurofibromatosis type 1 (NF1) gene. The large size of the gene and relatively insensitive techniques has made detection of causative mutation difficult especially for this NF1 gene. As NF1 is one of the most common autosomal dominant disorders with a high mutation rate, it is of prime interest to medicine that an efficient and reliable method is available to diagnose this disease. Screening for NF1 mutations, particularly in neonates and young children who are often a/oligosymptomatic, is thus one of the major applications of the present invention. Since the NF1 gene is ubiquitously expressed, test samples of the subject can be obtained from a variety of tissues or blood. An NF1 test can also be included in panels of prenatal tests since NF1 DNA, RNA or protein can also be assessed in amniotic fluid and chorion villi. Further description of the invention will illustrate the technique starting from blood cells, nevertheless, explained principles can also be applied when starting from other cells.

Analysis can be performed on blood sent from any location such as private doctor practices or hospitals. Peripheral blood lymphocytes can be cultured for a short time, using phytohaemaglutinin stimulation. Alternatively, EBV transformed cell lines also allow repetitive analyses in a controlled environment.

Preferably, the present invention provides a method as defined above wherein said protein synthesis inhibitor might be chosen from a group comprising puromycin, actinomycinD, cycloheximide or a possible analogue thereof. All of these prevent nonsense mediated decay of the mutant transcript. Mutations can be missed starting from a culture without puromycin treatment, due to the instability of the mutant transcript and the “premature termination codon induced” mRNA decay. Puromycin is preferred in the proposed method. Puromycin is a tRNA analogue causing chain termination and blocks nonsense-mediated decay in cell lines as was demonstrated to be the case for the hMSH2 gene (Andreutti-Zaugg et al., 1997). The effect of puromycin on the stability of the NF1 mutant transcript was not experimentally investigated before. The present inventors proved for the first time that the addition of puromycin to cell cultures could also improve the analysis of mutant transcripts of the NF1 gene thereby increasing the efficiency of NF1 genetic diagnosis.

Until now, for the NF1 mutation analysis no indication was available when RNA isolation should be started. The inventors point out that production of newly made NF1 messenger RNA in specific conditions is of prime importance. Indeed, according to present invention it is essential that RNA is extracted immediately from the cultures of said EBV transformed B-lymphoblastoid cell lines or short-term cultures of PHA stimulated blood lymphocytes once they are retrieved from the incubator. As shown by the inventors, incubation of cell cultures at room temperature will influence the splicing of the NF1 messenger creating alternatively spliced products which may influence the interpretation of the NF1 analysis. The inventors showed that a crucial parameter in the successful application of the PTT to find the disease causing mutation is the quality of the RNA that is used to start the procedure. It was noticed that starting from RNA extracted from peripheral blood cells, kept for a while at room temperature, very often “spurious” bands were present after RT-PCR as well as on the PTT SDS-PAGE. It is obvious for a skilled person in the art that this would also account for possible artifacts if one incubates said EBV transformed B-lymphoblastoid cell lines at room temperature. Therefore it is also suggested by the inventors that RNA should be immediately extracted from the cell lines once they are removed from the incubator. The incubator creates an optimized environment for cell growth with stable CO₂ pressure and temperature (37° C.). Consequently, if cells are immediately analyzed after removal, epigenetic factors will not have the time to influence the activation of cryptic splice sites leading to the occurrence of these deleted transcripts. The inventors showed that RNA extracted from “aged” blood samples leads to increased skipping of exons and hence mimics—in the absence of a genomic alternation—the presence of a mutation. This results in a wrong interpretation of the genomic background of the patient, which cannot be allowed in medical diagnosis. Although it was already observed that infidelity of the splicing process could occur in specific gene transcripts of TSG101 and FHIT (Gayther et al., 1997) when RNA was isolated from “aged” blood, it seemed that this infidelity is gene specific and not a generalized phenomenon. Indeed this does not occur in other transcripts of other tumor suppressor genes such as BRCA1, BRCA2, BRUSH1, hMSH2, IGF2 receptor, PGDβ and RB (Gayther et al, 1997). Therefore it was not evident to find this phenomenon for the NF1 gene.

According to a preferred embodiment, the present invention also relates to a method as defined above wherein RNA is immediately extracted from immediately isolated peripheral blood lymphocytes of said patient for further analysis of the mutations present in the DNA of said patient (FIG. 1). Also in this case epigenetic factors would not have the time to influence the activation of cryptic sites. The term ‘immediately’ implies not longer than 2 hours after blood collection and preferably as soon as possible. Often RNA cannot be extracted immediately after prelevation of the blood, which is often the case in clinical practice when samples are sent from abroad, lymphocytes are revived by short term (48-168 hours) culture using phytohaemagglutinin (PHA) stimulation at 37° C. Short-term culture moreover allows to obtain a much larger cell population for the extraction of the RNA.

Preferably, the methods according to the present invention involve a reverse transcriptase (RT) step followed by an amplification step, which is a polymerase chain reaction (PCR) (FIG. 1). This step allows the amplification of gene fragments covering the whole coding domain of the NF1 gene, which makes the analysis of a gene consisting of multiple exons more feasible.

Preferably, the present invention provides a method as defined above wherein said RNA extracted in step d) is total RNA. Isolation of total RNA is less expensive compared to the isolation of mRNA and will still result in the amplification of specific gene products when amplification conditions and primers are chosen appropriate as described by the method. Amplification products can be used to verify the corresponding DNA sequence or used to produce corresponding proteins (FIGS. 1 and 2).

The said PCR products can be used in an in vitro translation system, so NF1-peptide fragments can be made. In addition, the present invention preferably provides methods as defined above wherein step f) is followed by a separation of said peptide fragments. This separation can be done by any technique known in the art such as SDS PAGE (one or two dimensional). In previous experiments, as described by Heim et al. (1995) and Park et al. (1998), isotopic ³⁵S-Methionine is incorporated in the peptides so separated peptides can be easily visualized using radiography. Contrarily to the prior art, the inventors changed the label to ³H-Leucine. Changing the label increases the sensitivity of the mutation analysis significantly. This increase can be explained by the fact that the NF1 protein is rich in Leucine residues compared to the number of Methionines present in this protein and allows therefore a higher incorporation of specific label thereby increasing the detection efficiency. Alternatively to SDS-PAGE, mass spectrometry or Malditoff can be used to analyse the peptide population obtained.

The present invention also provides a method that is as sensitive that it is possible to identify the NF1 mutation in sporadic patients presenting as somatic mosaic. More precisely, the sensitivity of the test allows to detect the NF1 mutation if present in at least 10% of the cells that are under investigation (FIG. 17). Up to now, somatic mosaicism could only be detected via FISH analysis and only for patients carrying large deletions. Other studies failed to identify the mutation with equal efficiency in sporadic patients (Ars et al. (1995): 51% detection rate in sporadic patients; Fahsold et al. (2000): 53% detection rate without specification between sporadic versus familial; our data: 83% detection in sporadic cases by PTT alone). So, by increasing the detection efficiency we are able to detect this aberration even when a point mutation is present. This is of uttermost importance as for sporadic patients only the identification of the pathogenic mutation allows for presymptomatic/prenatal diagnosis in future generations.

The present invention also provides a method as defined above wherein in case a truncated peptide is observed by means of protein separation, the amplified cDNA fragment obtained in step e) is analyzed by cycle sequencing allowing the characterization of the effect of the mutation at the mRNA level. It is not excluded that this amplified cDNA fragment can be analyzed without any hint given by such an in vitro PTT system. Moreover, comparison of the cDNA analysis from cells treated with and without puromycin allows to give information on the stability of the mutant mRNA in the affected cells. As stable mRNA may result in the production of a truncated neurofibromin, this information may point to novel putative functional domains in neurofibromin.

Preferably said analysis may be performed by means of cycle sequencing of a suitable fragment by means of suitable primers. From the length of the peptide fragment, it is mostly known which primers will be suitable. Primers for amplification of each of these fragments are known or can be readily developed (see also table 2 and 3 or any given table). Primers that are used for fragment amplification can also be applied to sequence respective amplified fragment. In this latter case, primers are labeled as known by a person skilled in the art.

Preferably such methods according to the present invention will employ as primers of step e) primers as represented in FIG. 2 or in any of the tables or in the Examples or figure legends.

Prefered methods according to the present invention employ non-isotopically labeled primers. Said label is chosen from a group comprising fluorescein, biotin, Cy5, FAM6, TAMRA, ROX.

The generated cDNA fragments may be further analyzed by means of ALF-sequencer (Pharmacia), ABI-370 (Perkin Elmer) or any other sensitive semi- or automatic sequencing system.

Currently, no lab in the world performs routine NF1 mutation diagnostic assays for large numbers of patients. One of the reasons for this is the inability to find high sensitivity and high specificity methodology for routine diagnostic testing of NF1 gene mutations. It is therefore highly desirable to have an improved diagnostic methodfor the presence or absence of NF1 mutation.

By the present optimized Protein Truncation Test >83% of the germline mutations in NF1 patients fulfilling the N.I.H. diagnostic criteria can be identified. The spectrum of mutations that can be detected by PTT is limited to nonsense mutations, frameshift mutations, splice mutations, deletions not encompassing the region flanked by the used primers, all leading to a premature termination codon. Additional methods are needed to detect missense mutations, small (less than 80 nucleotides) in frame deletions and/or insertions, large deletions and cytogenetic abnormalities such as translocations.

The method of the invention relates to a hierarchical system for effective molecular diagnosis of NF1 disease-associated mutations. The spectrum of mutations reveals the high incidence of unusual splice mutations. Many of these mutations will be missed using genomic scanning techniques as many splicing mutations are caused by intronic mutations outside the canonical splice donor/acceptor sequences. Moreover, some mutations called “silent” at the genomic level, create a novel splice donor or acceptor site and are proven to be pathogenic by the RNA-based mutation detection methods.

In the hierarchical system, the second level of analysis for patients that score negative with the optimized PTT system includes methods to detect missense mutations and/or small in frame insertions and/or deletions. These analyses can be performed by means of heteroduplex analysis (HA) and/or single stranded conformation polymorphism (SSCP) analysis and/or denaturing gradient gelelectrophoresis (DGGE) and/or conformation sensitive gelelectrophoresis (CSGE) and/ or immediate cycle sequencing (with or without subcloning).

Said HA or single stranded confirmation analysis is performed to detect aberrant migrating PCR fragments which are then further analyzed by cycle sequencing.

A preferred combined approach for the characterization of an NF1 germline mutation according to the present invention involves a protein truncation test from EBV transformed cell lines as detailed above and in the examples and figure legends followed by direct cDNA and gDNA sequencing, heteroduplex analysis followed by direct gDNA sequencing, Southern blot analysis using probes GE2-FF13-FF1-FB5D-AE25-P5-B3A as described in Marchuk et al, 1991 (These clones were a kind gift of Francis Collins), FISH (fluorescence in situ hybridization) analysis using intragenic cosmid or PAC clones and cytogenetic analysis.

Preferably, the present invention provides a method for mutation analysis of the NF1 gene of a patient as defined above wherein said primers are located flanking exon 4b, 7, 10a-10c, 13, 23.2, 27a, 29, 37 or 39 of the NF1 gene respectively, as represented on FIG. 7 and 8. We have found that the mutation spectrum of NF1 and the identification of mutational hotspots as defined before was biased by the limitations of the technology that was used as well as by the fact that the total coding region of the NF1 gene was not screened in older studies. We describe as examples thereof several hotspot domains for mutations in the NF1 gene which were not identified before: i.e. exon 7, exon 10a-10b-10c, exon 13 (2033insC), exon 23.2 (R1362X), exon 27a (R1513X), exon 29 (R1849X), exon 39 (2266delNF). Assay kits for screening and diagnosis of mutations within these specific novel hotspots in accordance with the principles of the present invention are also provided. Focusing on these domains will improve the speed in which mutations can be diagnosed.

The present invention also provides a method for detecting previously published mutations as well as the following novel specific frame shifts, nonsense or splice mutations (see table 1): K33K (99del105), C93Y (278G>A), C187Y (560G>A), R192X (574C>T), 603-604insT (idem), Q209X (625C>T), 819-821delCCT (idem), 889-454del474nt(888del174), 987-988insA (idem), 1261-19G>A (1260insTTTGTTTTTCTCTAGTC (SEQ ID NO 1)), W425X (1275G>A), R461X (1381C>T), Y489C (1465del62), 1466insC (idem), 1527+5G>A (1392del135), E524X (1570G>T), 1605insA (idem), S536X (1607C>A), 1642-3C>G (1641del80), 2305insT (idem), 2585insA (idem), 2836insT (idem), 2850+2del6 (2617del233), 2851-6del4 (2850del140), Q959X (2875C>T), Q963X (2887C>T), 2990+3A>C (2850del140), Y1044X (3132C>A), 3193delC (idem), V1093M (3277G>A/ 3274 del40), 3108-3C>G (3314del182), E1123X (3367G>T), 3457delCTCA (idem), Q1174X (3520C>T), 3704delA (idem), 3708+1G>C (3496del212), 4026delG (idem), 4299delC (idem), Q1494X (4480C>T), 4515-2A>T (4515-14ins14/4515-17ins17), 4773-2A>T (4772del433/ 4772del293), 5033delG (idem), 5117delT (idem), R1849 (502del341/5205del544), S1755X (5264C>G), S1765X (5215del90/5294C>A), 5567delT (idem), 5798delC (idem), Q1966X (5896C>T), 6577delGAGgta (6364del215), R2237X (6709C>T), 6858G>C (6756del102), 7127-12T>A (7126del132/7127-10ins10), 7268delCA (idem), K2401X (7201A>T), R2429X (7285C>T), 7884-7885delGT (idem), 8016delA (idem). Nomenclature for the mutations found for the NF1 protein is as recommended by Antonarakis (1998). Effect of the mutation at the mRNA level is between parentheses. The letters and numbers refer to the mutation at the amino acid level which is mentioned first and the effect of said mutation at the mRNA level is mentioned in the parentheses or the genomic mutation t(14;17)(q32;q11.2) interrupting the NF1 gene. All mutations were verified to be present at the genomic DNA level. The novel balanced translocation t(14;17)(q32;q11.2) interrupted the NF1 gene: PAC928b9 was found on the der(17) and PAC1002g3 was found on der(14).

The present invention identifies a number of regions in the NF1 gene that can be skipped “in frame” by specific mutations in the genomic DNA and result in the production of a stable mRNA. A number of missense mutations were also identified. As both types of mutations may lead to the production of a truncated/altered neurofibromin, these mutations may point to novel functional domains of neurofibromin.

Thus far, only the central GAP-related domain has been well characterized (GRD in FIGS. 7 and 8). A region involved in cAMP-mediated signaling exists in Drosophila and probably in humans as well but its location in the NF1 gene is not yet defined. Neither has the region that mediates the association of neurofibromin to the microtubules been defined. Careful mutation analysis like the study presented here may point to the regions involved in these and other functions of the NF1 gene. Specifically the following regions may be important and are first described now: In frame skipping of the last 105 nt of exon 2 (mediated by K33K), C83Y (exon 3), C93Y (exon 4b), 274delL (exon 6), In frame skipping of exon 10b (mediated f.i. by 1527+5G>A), In frame skipping of both exon 11 and 12a (mediated f.i. by IVS12a+1G>T), L847P (exon 16), In frame skipping of the first 90 nt of exon 29 (mediated by f.i. S1765X), In frame skipping of exon 37 (mediated f.i. by 6792C>A, 6792C>G, K2286N), in frame skipping of exon 40 (mediated f.i. by IVS39-12T>A). The established EBV-cell lines harboring these specific mutations are valuable tools to perform further protein research to define additional functional regions onto the NF1 protein, e.g., specific transcripts can be isolated from these EBV cell lines and used to setup two-hybrid screenings.

The present invention also relates to a diagnostic kit for mutation analysis of the NF1 gene of a patient comprising primers specifically amplifying the gene domains containing the novel specific mutations or the novel mutation hotspot regions as mentioned above.

The present invention also relates to a diagnostic kit for mutation analysis of the NF1 gene of a patient comprising probes specifically detecting the gene domains containing novel mutation hotspots regions or specific mutations as mentioned above.

The term “nucleic acid” refers to a single stranded or double stranded nucleic acid sequence present in a biological sample, said nucleic acid may consist of deoxyribonucleotides or ribonucleotides or may be amplified cDNA or amplified genomic DNA.

The term “probe” refers to single stranded oligonucleotides and may consist of deoxyribonucleotides or ribonucleotides, nucleotide analogues or modified nucleotides, or may be amplified cDNA or amplified genomic DNA.

The probes used in the process of the invention can be produced by any method known in the art, such as cloning of recombinant plasmids containing inserts including the corresponding nucleotide sequences, if need be, by cleaving the latter out from the cloned plasmids upon using the appropriate nucleases and recovering them (e.g., by fractionation according to molecular weight). The probes can also be synthesized chemically, for instance, by the conventional phopho-triester method.

The probes of the invention can optionally be labelled using any conventional label. This may include the use of labelled nucleotides incorporated during the polymerase step of the amplification or by any other method known to the person skilled in the art.

The term “primer” refers to a single stranded nucleotide sequence capable of acting as a point of initiation for synthesis of a primer extension product which is complementary to the nucleic acid strand to be copied. The length and the sequence of the primer must be such that they allow to prime the synthesis of the extension products. Preferably the primer is about 5-50 nucleotides. Specific length and sequence will depend on the complexity or the required DNA or RNA targets, as well as on the conditions of primer use such as temperature and ionic strength.

The present invention also advantageously provides nucleic acid sequences of at least approximately 15 contiguous nucleotides of the NF1 gene or mutant versions thereof, preferably from 15 to 50 nucleotides. These sequences may, advantageously be used as probes to specifically hybridize to sequences of the invention as defined above or primers to initiate specific amplification or replication of sequences of the invention as defined above, or the like. They may also be used in diagnostic kits or the like for detecting the presence of a nucleic acid according to the invention. These tests generally comprise contacting the probe with the sample under hybridizing conditions and detecting the presence of any duplex or triplex formation between the probe and any nucleic acid in the sample.

Identification of specific mutations in the NF1 gene also has therapeutic implications. A method for identifying a compound correcting the defective structure of the mutated NF1 protein is one of the examples. A mutated NF1 protein can result from a specific mutation of the NF1 coding region as described above. Modulation of NF1 function can be accomplished by the use of therapeutic agents or drugs. These can be designed to interact with different aspects of the NF1 protein structure or function; a drug can correct its defective structure or increase its affinity for a substrate or cofactor. Efficacy of a drug or agent can be identified by a screening program in which nodulation is monitored in vitro using cell systems in which a defective NF1 protein is expressed. Alternatively, drugs can be designed to modulate NF1 activity from knowledge of the structure correlation of the NF1 protein and from knowledge of the specific defect in the various NF1 mutant proteins (Capsey et al., 1988).

This invention also relates to model systems comprising an NF1 gene mutation, as defined above, which can be used to screen for therapeutic agents. In both in vitro and in vivo models mutant NF1 proteins are expressed and used to screen for correction of the mutant NF1 activity. In the in vitro tests both purified NF1 protein or cell lines expressing the mutant NF1 protein can be used; in the in vivo models, transgenic animals expressing the mutant NF1 protein can be employed.

Transgenic mice carrying a mutation in one of the NF1 genes show clear pathological symptoms. Vogel et al. (1999) described that cis-Nf1^{±}:p53^{±} mice exhibit a significant incidence of soft tissue sarcomas. The presence of the heterozygous NF1 mutation accelerates tumorigenesis and alters the tumor spectrum in the content of the p53^{±} background. In addition, chimeric mice composed in part of Nf1^{-/-} cells carrying homozygous NF1 alterations do develop neurofibromas (Cichowski et al., 1999). Consequently, both mouse models provide the means to test therapeutic strategies.

The following examples merely serve to illustrate the invention and are by no way to be understood as limiting the present invention.

### BRIEF DESCRIPTION OF THE DRAWINGS

FIG. 1: Overview of the optimized NF1 genetic mutation analysis. Peripheral blood lymphocytes are taken from the patients and DNA is extracted from the lymphocytes. Concomitant an EBV-transformed lymphoblastoid cell line is initiated or a short term culture of PHA stimulated lymphocytes is started. Once the culture is well established, the culture is split and 1 culture (P+ culture; wherein puromycin is added) is incubated with 200 μg/ml puromycin for 16 hours, the second culture (P− culture; wherein no puromycin is added) is further incubated in the RPMI 1640 culture medium. In order to get a good signal to noise ratio for the cycle sequencing of cDNA, total RNA from the P+ culture is extracted. cDNA is prepared using random hexamers and the coding region is amplified in 5 overlapping fragments using a modified forward primer containing a T7 promotor sequence, the KOZAK sequence and a methionine start codon in frame with the sequence to be analyzed. Afterwards in vitro transcription/translation peptide fragments are separated by SDS-PAGE. If a truncated peptide is present, the RT-PCR fragment leading to this truncated peptide is analyzed by cycle sequencing. Once the cDNA sequence pattern has been interpreted we continue the analysis at the genomic level by cycle sequencing of the DNA extracted directly from the lymphocytes covering the whole coding domain of the NF1 gene. Hence, it can be excluded that the mutation that is identified was introduced due to the EBV transformation itself.

FIG. 2: Schematic overview of the total coding region of the NF1 gene (60 exons drawn to scale) and position of the 5 overlapping RT-PCR fragments used for in vitro transcription/translation. The position of the 5 overlapping RT-PCR fragments is denoted. Exon numbers are indicated. Overlap between the neighboring fragments is indicated in amino acids (AA). Small vertical bars indicate the position of the sequencing primers that are used to perform direct cycle sequencing of the RT-PCR fragments. Underneath are pictures from the SDS-PAGE showing the normal peptides obtained after in vitro transcription/translation of the 5 fragments. On every gel a protein marker (¹⁴C Methylated proteins CFA626, Amersham) is loaded. 69 kDa denotes the size of the largest peptide band from the protein marker. Smaller fragments of the protein marker are 46 kDa, 30 kDa and 14.3 kDa in size. GRD denotes the Gap-related domain, this is a domain with catalytic GTPase-stimulating activity (Kim and Tamanoi,1998). The GRD spans the amino acids 1172-1538 of the protein.

FIG. 3: Influence of puromycin on the analysis of NF1 exon 20. RNA was extracted from an EBV transformed lymphoblastoid cell culture with and without incubation with 200 μg/ml puromycin for 15 hours.

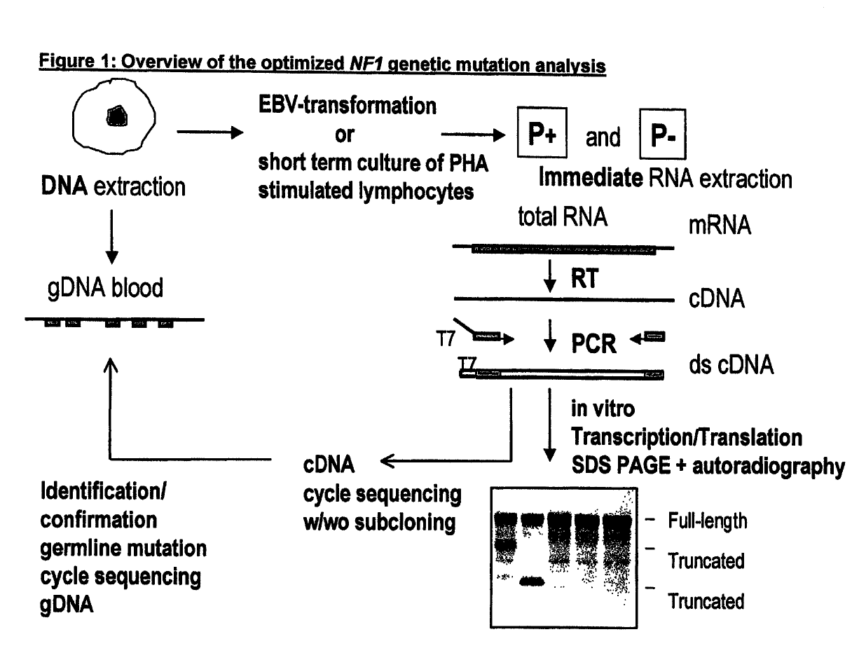

A. PTT from Pplus and Pmin EBV cultures from NF1 patient NF-039. Lane 1: protein markers (sizes in kDa). Lanes 2, 3, Pplus and Pmin cultures with and without puromycin treatment, the presence of a truncated peptide was discerned by PTT analysis. The intensity of the truncated peptide compared to the wild type peptide was greatly enhanced after puromycin treatment.

B. Starting from the cultures that were not treated with puromycin, direct cycle sequencing of the cDNA exon 20 without subcloning was unable to identify unambiguously he presence of the nonsense mutation in exon 20, due to the nonsense-mediated mRNA decay.

C. Starting from the puromycin treated cultures, direct cycle sequencing of the cDNA without subcloning gave unambiguous results leading to the clearcut identification of a mutation in Exon 20: 3367GtoT, E1123X.

FIG. 4: Influence of puromycin on the analysis of NF1 exon 10c. PTT from Pmin and Pplus EBV cultures from a normal control and NF1 patient NF-033. Lane 1: protein markers (sizes in kDa). Lanes 2, 4, Pplus cultures. Lanes 3, 5,

A. Pmin cultures. Lanes 2 and 3: normal control showing only wild-type (WT) NF1. Lanes 4 and 5: patient NF-033 showing truncated peptide due to the presence of NF1 stop codon mutation at amino acid 524.

B. cDNA sequence chromatograms of patient NF-033 of Pmin (upper panel) and Pplus (lower panel) EBV cultures. Arrow, heterozygous peak: GAA>TAA at amino acid 524.

Another example illustrating the PTT results obtained starting from RNA extracted from EBV transformed lymphoblastoid cell cultures immediately after removal from the incubator and treated with and without puromycin. Both with and without puromycin treatment, the presence of a truncated peptide was discerned. The intensity of the truncated peptide compared to the wild type peptide was greatly enhanced after puromycin treatment. Sequencing results are only shown for the fragments leading to the peptides shown in lanes 4 and 5. Here too, starting from the puromycin treated cultures, direct cycle sequencing of the cDNA without subcloning gave unambiguous results leading to the clearcut identification of a mutation in Exon 10c: 1570GtoT, E524X. The mutation would again have been missed if only direct cycle sequencing would be performed starting from the culture without puromycin treatment, due to the instability of the mutant transcript and the “premature termination codon induced” mRNA decay. Due to this decay the mutant messenger is only present in small quantities and relevant seuence information is lost as it does not peak above the background/noise peaks.

FIG. 5: Automated Laser Fluorescence (ALF) based fragment analysis of NF1 exon 7-skipping that is present in “aged” blood and in EBV cell lines carrying a specific nonsense mutation in exon 7, but that is not present in fresh blood nor in EBV cell lines not carrying a NF1 mutation in exon 7. FIG. 5 illustrates the importance of extracting the RNA immediately after prelevation for unprocessed blood or after removal from the incubator for cell cultures. Total RNA was extracted from Pplus and Pmin EBV cultures and from peripheral blood lymphocytes either processed immediately (“fresh”), or kept at room temperature and processed 48 hours after prelevation (“aged”) and semi-quantitative RT-PCR analysis of E7 skipping using a 5′-fluorescein labelled forward primer in E6 (5′-TTGACTTGGTGGTGGTTT-3′ (SEQ ID NO 2)) and a reverse primer in E8 (5′-TTGAGAATGGCTTACTTGGA-3′ (SEQ ID NO 3)). Lane 1, fresh peripheral blood lymphocytes. Lane 2, “aged” peripheral blood lymphocytes. Lane 3 and 5, Pmin EBV culture from patient NF-027 (A) and NF-064 (B) both carrying the mutation R304X. Lane 4 and 6, Pplus EBV culture from patient NF-027 (A) and NF-064 (B) both carrying the mutation R304X. Lane 7 and 8, Pmin (lane 7) and Pplus (lane 8) EBV culture from patient NF-019 carrying the mutation R2264X.

Fragment analysis of RT-PCR products using a 5′ fluorescein labeled forward primer located in exon 6 and a reverse primer located in exon 8. 20 cycles of amplification were performed, i.e. the exponential phase of the PCR well before a plateau is reached. Hence the experiments are semi-quantitative. RT-PCR fragments were separated on a 5% denaturing polyacrylamide gel on an ALF automated DNA sequencer (Pharmacia). Lengths of the fragments were evaluated with Fragment Manager software using internal and external markers (M). The quantity of each transcript was determined as the area under the curve, which was also sized by means of Fragment Manager software (Pharmacia) and normalized against the sum of all the fragments obtained for a particular sample. This is expressed as skip/total.

The analysis showed that ˜9% of the transcripts in “aged” blood (48 hours at room temperature) did not contain the exon 7 (exon 7 skipping) (lane 2).

Patient A and B, both carrying an identical nonsense mutation in the exon 7 showed a slightly higher level of exon 7 skipping in their transcripts (lane 3 to 6).

Exon 7 skipping was not present (at least not in amounts that are detectable with the technology used) in fresh blood samples in which RNA was extracted immediately after prelevation lane 1), nor in EBV cell lines from patients that had no mutation in NF1 exon 7 (lanes 7 and 8) (patient C).

P+ denotes with puromycin treatment, P− denotes without puromycin treatment. M denotes internal size markers.

FIG. 6: ALF based fragment analysis of NF1 exon 37-skipping that is present in “aged” blood and in EBV cell lines carrying a specific nonsense mutation in exon 37, but that is not present in fresh blood nor in EBV cell lines carrying a NF1 mutation in another exon. FIG. 6 provides another illustration of the importance of extracting the RNA immediately after prelevation for unprocessed blood or after removal from the incubator for cell cultures. Fragment analysis of RT-PCR products using a 5′ fluorescein labeled forward primer located in exon 36 and a reverse primer located in exon 38. 20 cycles of amplification were performed. RT-PCR fragments were separated on a 5% denaturing polyacrylamide gel on an ALF automated DNA sequencer (Pharmacia). Lengths of the fragments were evaluated with Fragment Manager software using internal and external markers (M). The quantity of each transcript was determined as the area under the curve, which was also sized by means of Fragment Manager software (Pharmacia) and normalised against the sum of all the fragments obtained for a particular sample. This is expressed as skip/total.

The analysis showed that ˜14% of the transcripts in aged blood (48 hours at room temperature) did not contain the exon 37 (exon 37 skipping). P+ denotes with puromycin treatment, P− denotes without puromycin treatment. M: denotes internal size markers. Patient C, carrying a nonsense mutation in the exon 37 showed a significantly higher level of exon 37 skipping in his transcripts.

Exon 37 skipping was almost absent (˜2%) in fresh blood samples (lane 1) and not detectable in EBV cell lines from patients that had a mutation in another exon of NF1 (lanes 5 and 6).

FIG. 7: Distribution of the mutations identified by the protein truncation test (PTT) of the total coding region of the NF1 gene by analyzing 105 patients. The figure gives a schematic representation of the total coding region of the NF1 gene drawn to scale. In 85 of the 105 patients a mutation in the NF1 gene could be visualised using PTT (see Table 1). Exon numbers are indicated as described by Viskochil D. (1998). A mutational hotspot is defined by the occurrence of at least 2 independently arisen mutations in 2 unrelated persons at the same nucleotide. Examples are R304X (exon 7), R440X (exon 10a), R461X (exon 10a), Y489C (exon 10b), 2033-2034insC (exon 13), R1362X (exon 23.2), R1513X (exon 27a), R1849Q (exon 29), 2366delNF (exon 39). Also the finding of two different mutations at the same spot (e.g. 1465-1466insC and 1466A>G in exon 10b) is in itself indicative of a mutational hotspot (Cooper et al., 1998) Moreover, if the number of mutations identified in the number of patients analysed is weighted against the size of the exons, a number of mutation dense regions stand out: i.e. exon 7, 10a-10b-10c and exon 37. Exon 31 was previously described to contain a mutational hotspot, i.e. R1947X (Upadhyaya and Cooper, 1998). We only found this mutation once so far, but it was found already by several research groups and hence it is a recurrent mutation. The denomination of this mutation as a mutational hotspot may however be due to a bias, as till recently only limited portions of the gene were investigated by research groups and preferentially, groups searched in those regions where mutations were reported by others before.

FIG. 8: Distribution of all mutations identified so far by us using the PTT for the total coding region of the NF1 gene in 105 patients, and heteroduplex analysis, FISH analysis, Southern blot analysis and cytogenetic analysis in patients in which no mutation was identified by PTT. The heteroduplex analysis has not yet been completed for all exons in all patients that were negative in the PTT assay. So far, 6 interesting missense mutations and/or in frame deletions were disclosed: C93Y (exon 3), C187Y (exon 4b), 274delL (exon 6), L847P (exon 16), 991delM (exon 17), 2366delNF (exon 39). Their distribution is given on top of the bar representing the total coding region of the NF1 gene. Besides, a deletion of the total gene was found in 4 patients and 1 translocation t(14;17)(q32;q11.2).

All mutations identified so far by analysing all together 105 patients are summarized in Table 1.

FIG. 9A: Overview of the “mutation rich” regions in the NF1 gene. In our study exons 7, 10a, 10b, 10c and 37 stand up as particularly mutation-rich regions. In order to define the percentage of the coding region of a certain exon, we made the ratio between the number of nucleotides of a given exon and the number of nucleotides of the total coding region (i.e. 8457 nt). In the denominator the total coding region from ATG to TGA was taken, with the exception of the alternatively spliced exons 9br, 23a and 48a, in which no mutations have ever been found.

FIG. 9B: Overview of the recurrent mutations found in this study. Between parentheses is denoted: the exon that is prone to the recurrent mutation, number of patients found in this specific study with the specific mutation, whether the patients are sporadic (S) or familial (F). If 2 apparently unrelated patients with an identical mutation are found and patients are familial cases, haplotype analysis was performed to confirm that both patients are indeed unrelated and hence that the mutations arose independently.

FIG. 10. Illustration of the power of the current methodology detecting 2 different cryptic splice acceptors in IVS26, activated by mutation IVS26-2A>TT Genomic DNA, cDNA and subcloned cDNA sequence chromatograms of 4515-2A>T (IVS26-2A>T) in patient NF-044.

A. genomic DNA sequencing chromatogram showing presence of mutation 4515-2A>T.

B. starting from puromycin treated EBV cell lines, direct cycle sequencing chromatograms of the exon 26 & 27 region showed that besides normal transcipts containing exon 27 derived from the Wild Type allele, also 2 mutant transcript populations were present: a fraction contain an insertion of the last 14 nt of IVS26 (mutant transcripts) and another fraction contain an insertion of the last 17 nt of IVS26 (mutant transcripts). Mutant transcripts are formed due to use of two different cryptic splice acceptors in IVS26 (see Table 9).

C. cDNA sequencing chromatograms of the 2 mutant transcript populations after subcloning formed in patient NF-044. A fraction contains an insertion of the last 14 nt of IVS26 (left panel) and another fraction contain an insertion of the last 17 nt of IVS26 (right panel).

FIG. 11. Illustration of the power of the current methodology detecting 2 different misspliced transcripts that are formed due to presence of the mutation IVS39-12T>A. PTT, cDNA and genomic DNA sequence chromatograms of IVS39-12T>A in patient NF-005.

A. PTT results. Lanes 1 and 2: peptides synthesized in vitro from normal control EBV cultures. Lane 3: peptides synthesized in vitro from a Pmin EBV culture of patient NF-005. Arrowheads indicate presence of 2 different truncated peptides: one derived from transcripts in which exon 40 is skipped, another derived from transcripts formed by use of a novel splice acceptor in IVS 39 leading to insertion of the last 10 nt of IVS39.

B. cycle sequencing of the mutant cloned cDNA transcripts. Upper panel, transcripts with an insertion of the last 10 nucleotides of IVS39 due to use of the novel splice acceptor (gtttgtttgtttgtttagtttutagtag (SEQ ID NO 4)) created by 7127-12T>A. Transcripts lead to a truncated peptide of 209 amino acids after in vitro translation. Lower panel, transcripts with E40 skipping resulting in a peptide shortened by only 44 amino acids after in vitro translation.

C. cycle sequencing of the splice acceptor site genomic region of E40. Arrow, heterozygous peak showing the 7127-12T>A mutation.

FIG. 12: genomic and cDNA analysis of a patient with the mutation Y489C or 1466A>G in exon 10b resulting in the creation of a novel splice door site. Exon 10b of the NF1 gene: 1466A to G (Y489C) is a “missense mutation” masquerading a splicing mutation. The mutation creates a novel splice donor site that succesfully competes with the normal unaltered splice donor leading to skipping of the last 62 nt of exon 10b.

A. Cycle sequencing without subcloning of the genomic region of exon 10b in patient NF-017 showing transition of A to G at nt 1466 resulting in the formation of a splice donor site CT/gtaag;

B. Cycle sequencing of the mutant cloned cDNA allele in patient NF-017. The last 62-bp of exon 10b are skipped and immediately followed by exon 10c, resulting in the formation of a stop codon at amino acid 489;

C. Schematic diagram of the genomic region surrounding exon 10b. Shaded boxes represent exons, normal and novel splice donor sequences are denoted. By the transition of A to G at nt 1466 a novel splice donor is formed.

FIG. 13: genomic and cDNA analysis of a patient showing mutation V1093M or 3277G>A in exon 19b resulting in the formation of a novel splice donor site that is used by the splicing machinery. Exon 19b of the NF1 gene: 3277GtoA (V1093M) is a “missense mutation” masquerading a splicing mutation. The mutation creates a novel splice donor site that competes with the normal unaltered splice donor leading to skipping of the last 40 nt of exon 19b. Novel information is obtained on splice preferences in the NF1 gene using Splice Site Prediction using Neural Networks (http://www-hgc.lbl.gov/projects/splice.html). Evaluation of the sequences using this in silico prediction shows that wild type exon 19b contains a very weak splice donor and can be inactivated even by creation of another weak splice donor upstream.

A. Cycle sequencing without subcloning of the genomic region of exon 19b in patient NF-063 showing substitution of G to A at nt 3277 resulting in the formation of a weak splice donor site TG/gtatg; see Table 9

B. Direct cycle sequencing of the mutant cDNA alleles in patient NF-063. The last 40-bp of exon 19b are skipped and immediately followed by exon 20;

C. Schematic diagram of the genomic region surrounding exon 19b. Shaded boxes represent exons, normal and novel splice donor sequences are denoted. By the substitution of G to A at nt 3277 a novel splice donor is formed.

FIG. 14: 5294C>A (S1765X) nonsense mutation. Exon 29 of the NF1 gene: 5294CtoA (S1765X is a “nonsense mutation” masquerading a splicing mutation. The mutation creates a novel weak splice acceptor site that competes with the normal unaltered splice acceptor leading to skipping of the first 90 nt of exon 29. Novel information is obtained on splice preferences in the NF1 gene using Splice Site Prediction using Neural Networks (http://www-hgc.lbl.gov/projects/splice.html). Exon 29 contains a strong splice acceptor that however already can be inactivated even by creation of a weak splice acceptor downstream.

A. Cycle sequencing without subcloning of the genomic region of exon 29 in patient NF-009 showing substitution of C to A at nt 5294 resulting in the formation of a novel splice acceptor site; see Table 9

B. Direct cycle sequencing of the mutant cDNA alleles in patient NF-009. The first 90-bp of exon 29 are;

C. Schematic diagram of the genomic region surrounding exon 29. Shaded boxes represent exons, normal and novel splice acceptor sequences are denoted. By the substitution of C to A at nt 5294 a novel splice donor is formed.

FIG. 15: effect of nonsense mutations on splicing. PTT, cDNA and gDNA sequencing results of patient NF-027 with mutation R304X and PTT results of patient NF-003 and NF-019 with mutation Y2264X.

A. PTT results using primers encompassing NF1 exons 28-38. Lane 1 & 2 normal and truncated peptides formed in patients NF-003 (lane 1) and in patient NF-019 (lane 2). We identified the first nonsense mutations causing skipping of an exon containing these specific nonsense mutations (6792C>A and 6792C>G; published in Messiaen et al., 1997). Hoffmeyer et al. (1998) subsequently claimed that other nonsense codons in the NF1 gene are disparate splice effectors. We show using our detailed mutation analysis technology that only few nonsense mutations in the NF1 gene are inducing exon skipping, i.e; the formerly described 6792C>A and 6792C>G. We disagree with Hoffmeyer et al that the predominant effect of R304X is skipping of exon 7, see FIG. 15 B and C. Our experiments showed that the predominant effect of presence of R304X is formation of a stopcodon, as clearly evidenced on PTT assay and by cycle sequencing of the cDNA.

B. PTT results using primers encompasing NF1 exons 1-12a. Analysis of 2 patients with a truncated peptide using the abovementioned primers starting from EBV transformed lymphoblastoid cell cultures not treated with puromycin and immediate RNA extraction after removal from the incubator. Lane 2, 3, 4 are normal controls and show presence of the normally observed bands not further discussed or relevant in this figure. Lane 5 shows presence of a truncated peptide in another patient carrying a mutation in the region between exons 28-38. Lane 6 shows presence of a truncated peptide of approximately 33 kDa in patient NF-027. Lane 1: protein marker.

C. cycle sequencing without subcloning of (a) gDNA of patient NF-027 showing presence of the 910C>T substitution changing R304 into a stopcodon, (b) cDNA of patient NF-027 Pmin EBV culture. Arrow: presence of the TGA allele in a minor fraction of the transcripts (unequal expression), (c) cDNA of patient NF-027 Pplus EBV culture. Arrow: presence of the TGA allele present in equal amounts due to the inhibition of the nonsense mediated mRNA decay. 33 kDa is the size expected in the PTT reaction when the nonsense codon is retained and the optimized PTT could correctly identify the major effect of R304X on transcription.

FIG. 16: Use of the combined cascade of testing allows making distinction between (even very rare) polymorphism and bonafide pathological mutations. The evaluation of the pathological effect of missense mutations is very difficult in the absence of knowledge of all functional domains in a protein. Often missense mutations are reported as bona fide mutations although firm data underscoring these conclusions are missing (Lambert et al., 2000). We identified a missense mutation in exon 45 of the NF1 gene, R2616Q: this mutation was not found in 300 normal control chromosomes, the amino acid Arginine is conserved in Drosophila, mouse, rat and Fugu and change of arginine for glutamine is predicted to cause a dramatic change in the polypeptide chain . Still, the missense mutation did not segregate with the disorder in the family that was studied. By PTT of the total coding region the real pathogenic lesion was identified, i.e. R304X in exon 7, illustrating the strength of the technology. Efficient and correct molecular mutation analysis is extremely important, as the most immediate result of the current findings is the ability to provide presymptomatic and/or prenatal diagnosis.

FIG. 17: Somatic mosaicism for R2429X in a sporadic NF1 patient NF-075. Patient NF-075 is a male patient born in 1989 and has 2 small CAL spots (<5mm), subcutaneous neurofibromas supraclavicular and a plexiform neurofibroma surrounding the R atrium and septum and invading the pericard, and multiple internal neurofibromas in the mediastinum, freckling in left axilla and 2 isch noduli in left eye.

A. schematic representation of fragment 5, small bars denote the presence of sequencing primers to study this fragment, asterisk denotes the position of the stopcodon found in patient NF-075.

B. panel 1: PTT analysis from fragment 5 and separation of the peptides on a 15% SDS-PAGE and 20 hrs exposure of autoradiograms in 2 normal control cell lines treated with puromycin (lane 2, 3) , in patient NF-055 EBV cell lines treated with (lane 4) and without (lane 5) puromycin. This patient NF-055 carries the germline mutation R2429X. In patient NF-075 a weak truncated band at exactly the same position as the truncated peptide previously detected in another patient NF-055 was revealed. Only with an optimal signal to noise ratio it is possible to discern such a faint truncated band. PTT, starting from RNA extracted immediately after taking the EBV cell line from the incubator, and using the very sensitive ³H-Leucine incorporation, can effectively pinpoint the region of interest for further molecular study.

panel 2: PTT analysis from fragment 5 and separation of the peptides on a 10% SDS-PAGE and 20 hrs exposure of autoradiograms in cell lines from patient NF-055 (lanes 1 and 2) and patient NF-075 (lanes 2 and 4), treated with puromycin (lane 1 and 3) and without puromycin (lanes 2 and 4)

panel 3: same gel as in panel 2 but with a longer exposure time (60 hrs instead of 20 hrs). Using the longer exposure time the truncated peptide in patient NF-075 can be more readily seen.

C. (1) genomic DNA direct cycle sequencing chromatograms of NF-055 reveals presence of mutation R2429X in his blood lymphocytes. Equal quantity of mutant versus wild type allele is present, as can be expected for a germline mutation present on 1 NF1-copy in all cells; (2) genomic DNA direct cycle sequencing chromatograms of a normal control person; (3) Genomic DNA direct cycle sequencing of NF-075 reveals at that sequence the presence of a small signal that might indicate presence of a T nucleotide at position 7285 in a fraction of the cells. Cycle sequencing in itself is not sensitive enough to give any pathological significance to such a signal; (4) Further analysis of the genomic DNA of patient NF-075 by subcloning revealed presence of mutation R2429X in a fraction of his blood cells. This is the first sporadic patient that could be identified to be a “somatic mosaic” for a nonsense mutation in the NF1 gene. Fragment analysis (not shown) showed that the mutation is present in <10% of the blood cells.

### DETAILED DESCRIPTION OF THE INVENTION

### EXAMPLES

### Example 1

### Patients

From all patients EBV transformed B-lymphoblastoid cell lines and/or short-term phytohaemaglutinin stimulated blood lymphocyte culutes were established to allow for extensive RNA and DNA based studies. DNA was extracted directly from the lymphocytes as well. Patients were ascertained in the Center of Medical Genetics of Ghent, Brussels and Liege (Belgium). Before the analysis was initiated a detailed clinical evaluation was performed and the clinical features of all patients were documented using the “NNFF International NF1 Mutation analysis Consortium” form. Only patients that fulfilled the diagnostic criteria as proposed by the NIH Consensus Statement in 1988 (Stumpf et al.) and updated in 1997 (Gutmann et al.) were admitted to the study. The study was approved by the ethics committee of the University Hospital Ghent (Belgium).

### Example 2

### DNA Isolation, RNA Isolation and cDNA Synthesis.

EBV-transformed cell lines were grown in RPMI 1640. Prior to RNA isolation, the EBV transformed cell culture was split. In order to prevent nonsense mediated mRNA decay, one subculture was maintained in the presence of puromycin (16 hours, 200 μg/ml puromycin (Sigma, p7255), further called Pplus culture), while in the other subculture no puromycin was added (further called Pmin culture). RNA was extracted from both types of cultures for all patients.

Total cellular RNA was extracted with TRIzol LS Reagent (Gibco BRL, 10296-010) using the manufacturer's instructions.

cDNA was synthesized with 2-3 ug total RNA using random hexamers (Amersham Pharmacia Biotech) and 200 U Superscript II Reverse transcriptase (Gibco BRL).

### Example 3

### Protein truncation test

Primers used for the protein truncation test (PTT) assay have been described by Heim et al. (1995). PTT was performed using an optimized protocol (Claes et al., 1998). The sensitivity of the technique was further enhanced using puromycin treated EBV-transformed cell cultures and/or phytohaemaglutinin stimulated blood lymphocyte cultures and RNA extraction immediately after removal from the incubator. We analyzed all protein samples on a 10% and 15% SDS-PAGE gel in order to maximize the detection of the very large and small abnormal peptide fragments.

### Example 4

### Improved PTT Results

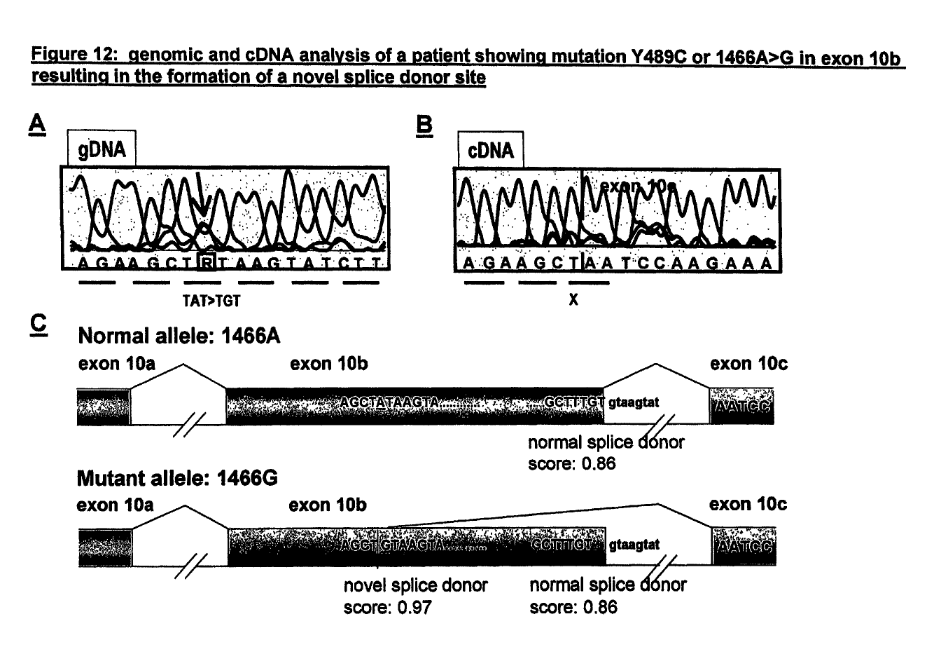

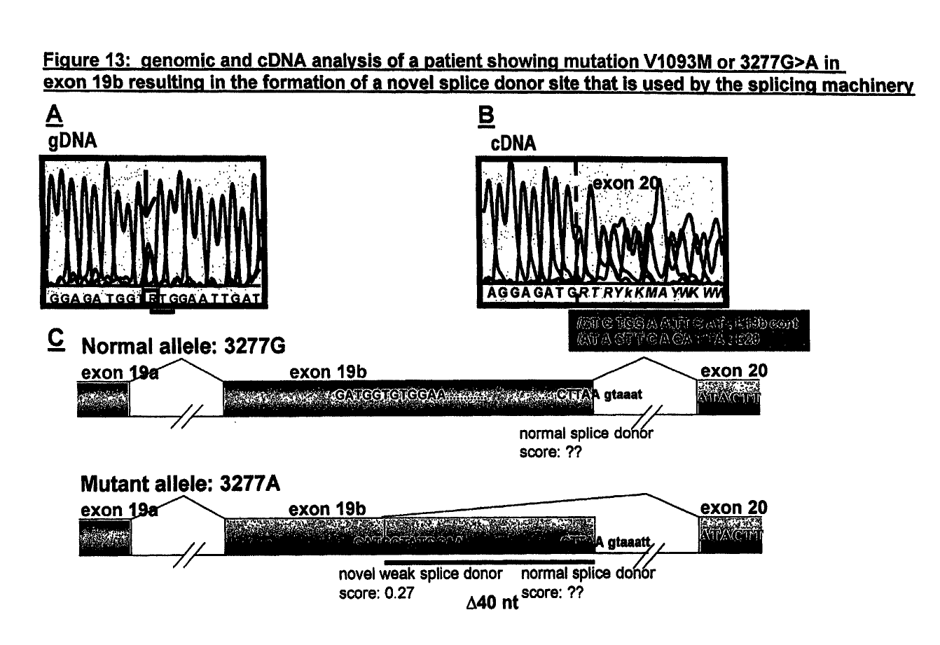

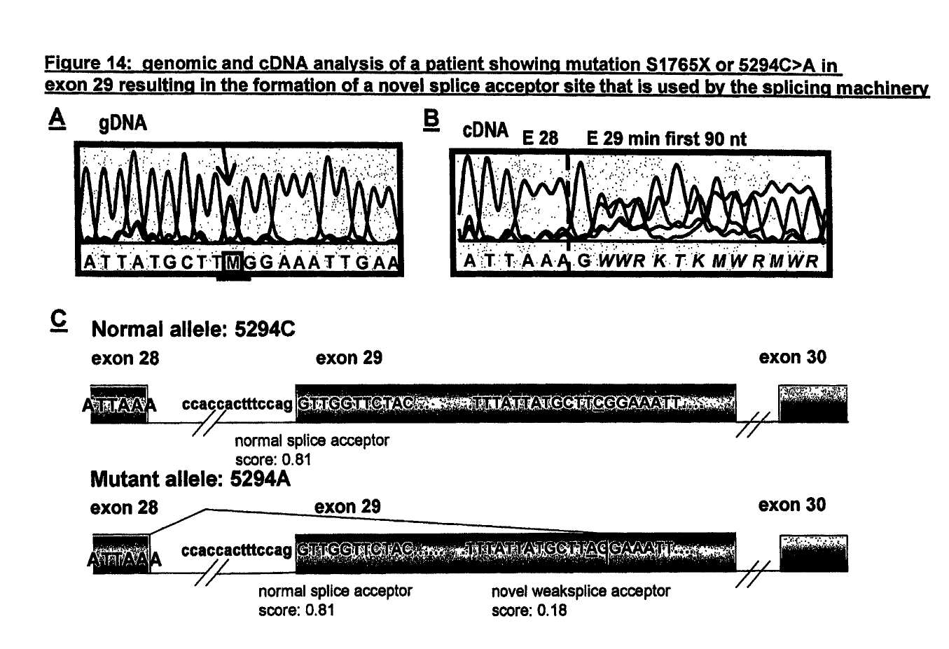

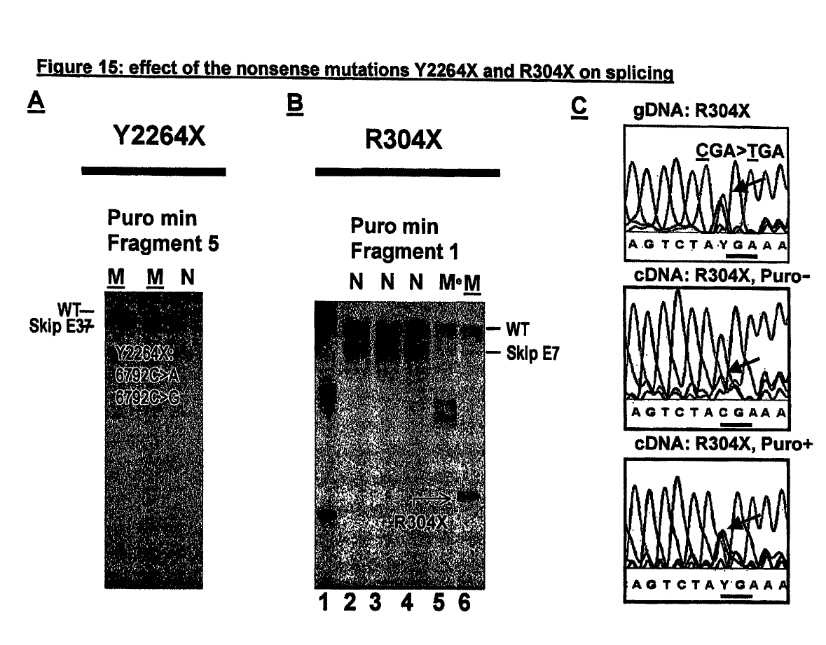

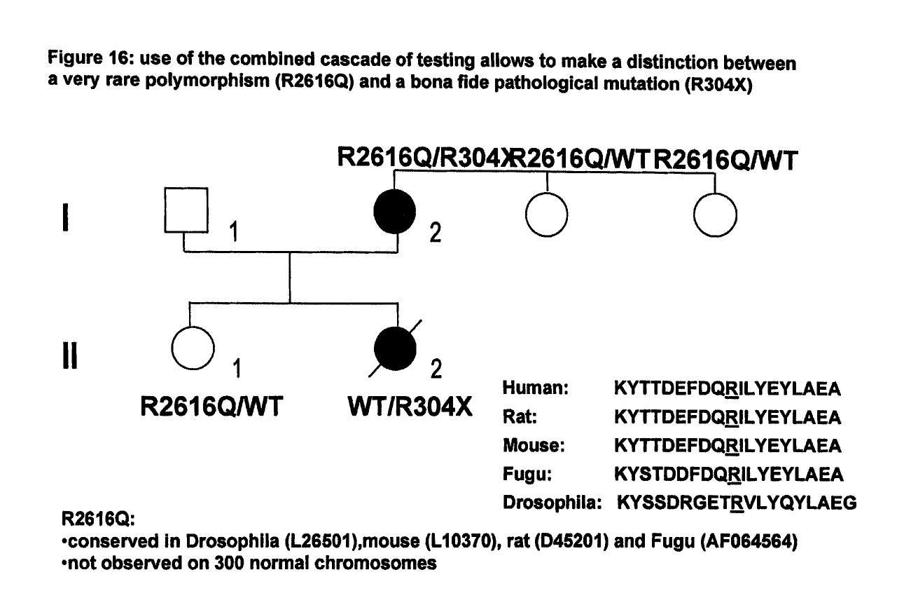

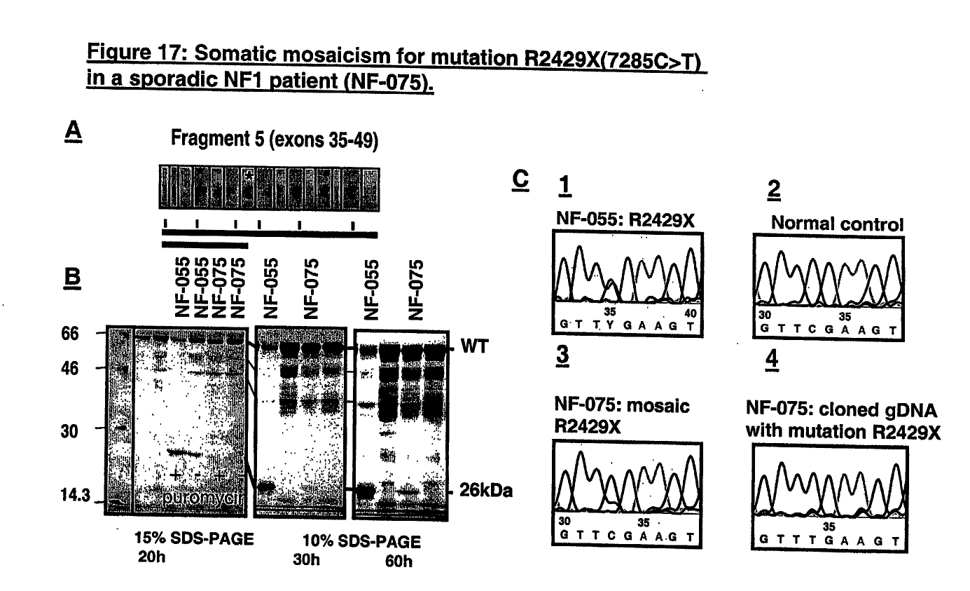

As the vast majority of mutations reported so far are predicted to generate a premature stop codon, it was chosen to start with the protein truncation test (PTT) in the first step of a combined mutation approach consisting of a cascade of techniques applied in the mutation analysis of the NF1 gene. However, the efficiency to detect truncating mutations by PTT depends on the stability as well as on the purity of the mutant mRNA under investigation. As nonsense-mediated mRNA decay has been documented in mutant NF1 alleles (Hoffmeyer et al, 1995) it was decided to also develop an optimized PTT for the NF1 gene using puromycin-treated EBV cell lines. Puromycin is a tRNA analogue causing chain termination and blocks nonsense-mediated decay in cell lines as was demonstrated to be the case for the hMSH2 gene (Andreutti-Zaugg et al., 1997). The effect of puromycin on the stability of the NF1 mutant transcripts was not investigated before. 67 EBV cell lines from NF1 patients were established. Established cell lines were grown until a T25 culture flask contained approximately 100 clusters with a diameter of 0.2-0.3 mm/cm² and were then divided in two separate wells (10 cm²): one well was treated for 15 hours with puromycin (200 μg/ml), the other well was further incubated in the RPMI-1640 tissue culture medium. Total RNA was extracted from both cultures and further analyzed by the protein truncation test. Although we did not miss a truncated peptide using RNA from the cultures without puromycin treatment—mainly due to the fact that a PTT system based on isotopic incorporation of ³H-Leucine is very sensitive—the direct cycle sequencing of all fragments leading to a truncating peptide was surprisingly greatly facilitated starting from cDNA prepared from the puromycin treated cultures (FIG. 3, 4 and 15). By the optimized PTT analysis, the germline mutation at the cDNA and gDNA level in 83% of the patients studied was succesfully identified.

Another crucial parameter in the successful application of the PTT to find the disease causing mutation is the quality of the RNA that is used to start the procedure. It was noticed that starting from RNA extracted from peripheral blood cells, very often “spurious” bands were present after RT-PCR as well as on the PTT SDS-PAGEs.

The origin of these spurious bands was further explored by comparing the transcripts obtained when RNA is extracted from the peripheral blood lymphocytes directly after prelevation or after 48 hours incubation at room temperature. The rationale for these experiments were twofold: i) in the natural situation the blood sample often stays for some time in the doctor's room and is transported hereafter to the lab. The time needed for this transport depends on the place from where the sample is referred, but can take more than 24 hours; ii) it has been reported that some tumor suppressor genes such as TSG101 and FHIT often show deletions at the cDNA level for which no mutations in the DNA can be found. Especially in tumor tissues and in “aged” (means that the blood sample was kept at room temperature for 60 hours before RNA was extracted) blood samples it was seen that both genes exhibit infidelity of the splicing process. Noteworthy, the authors observed that the breakpoints of the deleted transcripts coincide with cryptic splice donor or acceptor sites or with the skipping of entire exons. The splicing infidelity was observed to be gene specific and did not occur in other gene transcripts that were analyzed, i.e. BRCA1, BRCA2, hMSH2, IGF2, RB amongst others. Epigenetic factors may influence the activation of these cryptic sites leading to the occurrence of these deleted transcripts. Therefore, semi-quantitative experiments on the ratio of transcripts skipping of exons 7 and 37 compared to transcripts that do contain exons 7 and 37 were carried out. RNA was extracted from different sources, including fresh blood, “aged” blood at room temperature for 48 hours, EBV cell lines from normal control persons, EBV cell lines from patients harboring specific mutations in exon 7 and 37 leading to increased skipping of the respective exons (FIGS. 5 and 6). These experiments clearly demonstrated that RNA extracted from “aged” blood samples leads to increased skipping of these exons and hence mimics—in the absence of a genomic alteration—the presence of a mutation. The NF1 gene has to be considered to be a gene that is prone to alterations in the RNA processing in response to epigenetic factors.

As all missense mutations, small in frame insertions/deletions as well as large deletions and chromosomal rearrangements necessarily escape detection by the PTT analysis, all patients in which no mutation was identified in the first step were further analysed with a second battery of analyses. This second step included: DNA heteroduplex analysis of all 60 exons, FISH analysis using 3 intragenic cosmid/PAC clones, Southern blot analysis using 5 intragenic probes and finally karyotyping.

The combined approach surprisingly led to identification of the germline mutation in 64 out of 67 patients (>95%, Table 5). By PTT alone the germline mutation (after cDNA and genomic sequencing) could be identified in 56 out of the 67 patients analysed (83%). This is the highest detection rate reported so far.

This study indicates that 29% of the germline mutations in the NF1 gene are associated with aberrant splicing, a frequency that is much higher than that reported in surveys of other human genetic disorders (Krawczak et al., 1992 and Ruttledge et al., 1996), but is reminiscent of the situation found in the ATM gene (Teraoka et al., 1999). Given the fact that splicing errors as the cause for Neurofibromatosis type 1 are particularly frequent in the NF1 gene it is of paramount importance to avoid the occurrence of spliced transcripts that arise due to epigenetic factors such as incubation at room temperature of the blood lymphocytes outside their natural habitat (the bloodstream).

This can be obtained by working with EBV lymphoblastoid cell lines and extraction immediately after cultures are withdrawn from the incubator.

### Example 6

### Exon 10b of the NF1 gene represents a mutational hotspot and harbors a recurrent missense mutation Y489C associated with aberrant splicing

With the current technology several novel mutational hotspots were identified. One such a mutational hotspot resides in exon 10b. This region harbors a missense mutation that masquerades a splicing mutation.

Material and methods

NF1 patients

For all patients, the diagnosis of NF1 was based upon the presence of two or more of the diagnostic criteria proposed by the NIH Consensus Statement in 1988 (Stumpf et al., 1988) and updated in 1997 (Gutmann et al., 1997). The study was approved by the Institutional Ethical Committees and informed consent was obtained from the patients studied. Patients were recruited randomly without bias as they were seen for medical follow up and genetic advise. Patients were recruited as part of a general mutation study. 37 patients were contributed by the Dpt of Medical Genetics of Gent and by the Service de Genetique, Hopital Erasme Brussels.

Nucleic acid extraction

DNA and RNA samples were obtained from 37 unrelated NF1 patients by extraction from EBV-transformed lymphoblastoid cell lines. Total cellular RNA and genomic DNA was isolated as described (Messiaen et al., 1997).

cDNA analysis and in vitro transcription/translation analysis

First strand cDNA was synthesised by random priming (Messiaen et al., 1997) and cDNA was amplified using 5 primer pairs for amplification of the total coding region (10). 4 μl PCR product was used in an optimized in vitro transcription/translation reaction as described (Messiaen et al., 1997; Claes et al., 1998). An identical truncated peptide fragment of 55 kDa was observed in 2 out of 37 patients by in vitro transcription/translation of the fragment spanning exons 1 to 12a and the corresponding cDNA was analysed by cycle sequencing with and without subcloning using 0.15 μM fluorescein isothiocyanate (FITC) labeled primers, designated by nucleotide positions: 5′-CTTCGGAATTCTGCCTCT-3′ (SEQ ID NO 5) (400-418), 5′-CTGATATGGCTGAATGTG-3′ (SEQ ID NO 6) (719-736), 5′-GCCTGTGTCAAACTGTGT-3′ (SEQ ID NO 7) (967-984) and 5′- CACACCCAGCAATACGAA -3′ (SEQ ID NO 8) (1367-1384) and the Thermo Sequenase fluorescent labelled primer cycle sequencing kit (Amersham). Samples were loaded on a 6% LongRanger gel (FMC) containing 7M urea and analysed on an ALF automated DNA sequencer. In order to check for the presence of the missense mutation Y489C in a fraction of the cDNA, RT-PCR fragments were cloned using the pCR-TOPO cloning kit (Invitrogen) and 90 individual clones were further analysed by cycle sequencing.

Genomic DNA analysis

Exon 10b was amplified using the primer pair as described (Purandare et al., 1994) and PCR products were further analysed by cycle sequencing without subcloning (Messiaen et al., 1997). Mutations are reported according to the recommendations of the Nomenclature Working Group (Antonarakis, 1998), with the start site of translation denoted as nucleotide 1 both for cDNA and genomic alterations.

Results

The total coding region of the NF1 gene was analysed by the protein truncation test in 37 unrelated NF1 patients from which an EBV lymphoblastoid cell line was available (Heim et al., 1995). In 2 patients an identical shortened fragment of approximately 55 kDa was discerned in the region encompassing the exons 1 to 12a. In both patients in vitro transcription/translation for the other regions only showed normal sized fragments. By electrophoresis of the RT-PCR fragments from patient 1 two discrete bands were discerned on a 1.5% agarose gel, i.e. a normal sized band of 1868-bp and a band that was approximately 60-bp smaller. In patient 2 however only a normal sized band was seen, indicating that the truncated protein of identical size was formed in a different way in this patient. cDNA sequencing in this region indeed revealed a different mutation in both patients. In patient 2, an insertion of C at nt 1465-1466 in exon 10b was found, immediately resulting in the creation of a stop codon at this site. In patient 1, a deletion/skipping of the last 62 nucleotides of exon 10b was observed in RNA from both lymphocytes and the EBV-lymphoblastoid cells (FIG. 12B). Here too, the immediate result is formation of a stop codon at this site, explaining the identical picture seen by protein truncation analysis. Further analysis of exon 10b at the genomic level confirmed the presence of an insertion 1465insC in patient 2. In patient 1 however, a missense mutation was identified: A1466G, changing the codon for Tyr to Cys (Y489C) (FIG. 12A). Both parents of this sporadic patient did not carry this missense mutation. This missense mutation masquerades a splicing defect: indeed substitution of A to G at position 1466 of the genomic DNA creates a new splice donor site (CT/GTAAG) (FIG. 12C).

Analysis of the normal and mutant sequence using the program for splice site prediction by neural network (http://www-hgc.lbl.gov/projects/splice.html) showed a 0.86 score for the normal exon 10b donor site (GCTTTGT/gtaagtat (SEQ ID NO 9)) and a higher 0.97 score for the new donor site created by the missense mutation Y489C (AGAAGCT/gtaagtat (SEQ ID NO 10)). RT-PCR fragments from an EBV lymphoblastoid cell line of patient 1 were cloned and 90 individual clones were further analysed by cycle sequencing in order to check for the presence of the missense mutation in a fraction of the cDNA. In 50 cDNA clones showing a normal sized band of 1868-bp on a 1.5% agarose gel, only the wild type sequence was found and in none of them the missense mutation was present. In 40 clones containing a slightly smaller insert (approximately 60-bp) as evidenced by agarose gel electrophoresis, the smaller size was due to the skipping of the last 62 nucleotides of exon 10b along with intron 10b in the mRNA (FIG. 12B). This indicates that the major outcome of the mutation Y489C at the cDNA level is skipping of the last 62 nucleotides of exon 10b. Although Y489C and 1465-1466insC are different mutations at the genomic DNA level, both result in the formation of a premature stop codon at exactly the same position well before the GAP domain of neurofibromin. As the finding of two different mutations at the same spot (i.e. 1465-1466insC and 1466A>G) is in itself indicative of a mutational hotspot (Cooper et al., 1998) these findings prompted us to analyze exon 10b in a larger patient population. We now identified the mutation Y489C in 4 unrelated NF1 patients on a total number of investigated proven NF1 patients of 105, allowing to estimate prevalence of this mutation in the NF1 population to be about 4%.

### Example 7

### Exhaustive mutation analysis of the NF1 gene allows the identification of 95% of mutations and reveals a high frequency of unusual splicing defects.

Patients, Materials and Methods

Patient samples

In this prospective study, 67 unrelated index patients seen at the Departments of Medical Genetics of Ghent University Hospital, Université de Liège, Vrije Universiteit Brussel and Université Libre de Bruxelles for clinical follow-up and genetic counseling were included. Clinical features of all patients were documented using the “NNFF International NF1 Mutation analysis Consortium” form. Only patients fulfilling the diagnostic criteria as proposed by the NIH Consensus Statement in 1988 (Stumpf et al., 1988) and updated in 1997 (Gutmann et al., 1997) were included. The ethics committee of the Ghent University Hospital approved the study. From all patients EBV transformed B-lymphoblastoid cell lines were established. 38 patients presented as de novo cases and 29 patients were familial. The familial or sporadic nature of the mutation was verified by analysis of family members. All mutations were verified on a second independent sample. The mutations were absent on 100 unrelated normal chromosomes.

RNA Isolation and cDNA Synthesis

Prior to RNA isolation, the EBV transformed cell culture was split. In order to prevent nonsense mediated mRNA decay, one subculture was maintained in the presence of puromycin (16 hours, 200 μg/ml puromycin (Sigma, p7255), further called Pplus culture), while in the other subculture no puromycin was added (further called Pmin culture). RNA was extracted from both types of cultures for all patients.

Total cellular RNA was extracted with TRIzol LS Reagent (Gibco BRL, 10296-010) using the manufacturer's instructions.

cDNA was synthesized with 2-3 μg total RNA using random hexamers (Amersham Pharmacia Biotech) and 200 U Superscript II Reverse transcriptase (Gibco BRL).

RT-PCR and PTT

Primers used for the amplification of the total NF1 cDNA in 5 overlapping fragments (F1-F5) were as previously described (Heim et al., 1995). 3-5 μl PCR product, 20 μM amino acid mix minus Leucine (Promega, L4610) and 1.6 μl ³H Leucine (specific activity 1 mCi/mmol; Amersham) were added to the TNT™ Coupled Reticulocyte Lysate System (Promega). Reactions were performed as described (Claes et al., 1998). Samples were subjected to electrophoresis in a 10% and 15% SDS-polyacrylamide gel (Protean II Bio-rad, 20×24 cm gels) and run for 16 h at 30 mA (10% gels) and 40 mA (15% gels). ¹⁴C methylated protein (Amersham Pharmacia Biotech CFA626) was used as a protein-weight marker. Synthesized polypeptides were visualized by autoradiography after 20 and 60 h exposure to X-ray film.

Semi-quantitative analysis of splicing variants not associated with mutations

cDNA was subjected to 20 cycles of PCR using the following primer pairs: 5′-FITC-TTGACTTGGTGGATGGTTT-3′ (SEQ ID NO 11) (cDNA 749-777) and 5′-TTGAGAATGGCTTACTTGGA-3′ (SEQ ID NO 12) (cDNA 1096-1077) for analysis of exon 7 (E7) skipping; 5′-FITC- GGGCAGATAAAGCAGATAAT-3′ (SEQ ID NO 13) (cDNA 6721-6740) and 5′-CCGGATTGCCATAAATAC-3′ (SEQ ID NO 14) (cDNA 7029-7012) for analysis of E37 skipping.

Semi-quantitative analysis of the transcripts was performed on a 5% denaturing acrylamide gel on an ALF automated DNA sequencer (Amersham Pharmacia Biotech) as described (Lambert et al., 1998). The nature of shortened transcripts was verified after subcloning by direct cycle sequencing as described (Messiaen et al., 1997).

Splice site scores

The sequence environment of all splice mutations was analyzed using Splice Site Prediction by Neural Network (SSPNN) and a Splice Site Score (SSS) was obtained (URL adress: http://www.fruitfly.org/seq_tools/splice.html). For all 5′ and 3′ splice sites (ss) the consensus values (CV) were calculated as developed by Shapiro and Senepathy (1987).

cDNA sequencing

Autoradiograms from PTT gels allowed to predict the most plausible position of the mutations causing the specific truncated peptides and RT-PCR fragments were cycle sequenced in the corresponding regions with the Thermo Sequenase™ fluorescent labeled primer cycle sequencing kit (Amersham Pharmacia Biotech) using 5′-fluorescein or 5′-Cy5 labeled sequencing primers distributed along the coding sequence (sequences available upon request).

Heteroduplex Analysis (HA)

Exons were amplified from genomic DNA. For some exons PCR primers were developed using OLIGO V5 software (Table 6). For other exons PCR primers were as described (Purandare et al., 1994; Hoffmeyer et al., 1998, Maynard et al., 1997; Abernathy et al., 1997; Cawthon et al., 1990; Li et al., 1995). Exons 1 and 49 were not yet studied. For the larger exons 16, 21, 28, 29, 31, 33, 35, 37 and 38 the sensitivity of the HA was improved by digestion with a specific RE in order to obtain fragments with an optimal size between 200-300 nt. After amplification, fragments were denatured at 98° C. for 5′ and allowed to reanneal at 68° C. for 1 hour. 2-4 μl of the PCR product was mixed with 8 μl loading buffer (25% bromophenolblue, 25% xyleencyanol, 30% glycerol) and loaded on a 1 X MDE gel (FMC, Rockland, Me.) containing 10% glycerol. After electrophoresis, gels were stained with EtBr (0.5 μg/l) and evaluated under a transilluminator. Aberrant fragments were further analyzed by cycle sequencing using the forward amplification primer or a nested primer for sequencing.

Cytogenetic analysis and fluorescence in situ hybridisation (FISH)

Patients in whom no mutation was found by PTT and HA were analyzed by cytogenetic analysis and FISH. Cytogenetic analysis was performed on PHA stimulated G-banded metaphases according to standard procedures. To detect submicroscopic deletions dual color FISH was performed according to Van Roy et al (1994) using PAC clones 22 (926B9; 5′ NF1) and 13 (1002G3; 3′ NF1) described by Correa et al (1999).

Southern Blot analysis

6 μg of genomic DNA was digested with 30 U of EcoRI and BglII (both Gibco BRL) for 6 hours at 37° C. Digested DNA was electrophoresed in 0.8% agarose and transferred to positively charged Nylon membranes (Hybond N⁺ Amersham). Hybridisation was carried out using standard procedures (Sambrook et al., 1989) using cDNA probes GE2, FF13, FB5D, P5 and B3A as described (Marchuk et al., 1991).

Results

Mutation detection rate using the combined approach and mutational spectrum

We identified a bona fide pathogenic mutation in 64 of 67 unrelated NF1 patients (95.5%) (Table 5), including all 29 familial cases and 35 of 38 sporadic patients.

By the optimized PTT starting from puromycin-treated EBV-lymphoblastoid cell lines, the mutation was completely characterised both at the cDNA and gDNA level in 56 patients (56/67 patients; 83.5%): 25 were nonsense (25/67; 37%), 12 frameshift (12/67; 18%: 5 insertions and 7 deletions of one or a few basepairs) and 19 in-frame or out-of-frame splice mutations (19/67; 28%).

Further investigation of the remaining patients by HA resulted in the identification of 6 missense mutations and/or deletions of single amino acids (9%): C93Y, C187Y, L847P, 2970-2972delAAT or 991delM and 7096-7101delAACTTT or 2266delNF.

18 of the 44 (41%) single base-pair substitutions were due to a C>T or G>A transition at CpG dinucleotides, known to be prone to mutation if methylated.

A deletion of the entire NF1 gene was found by FISH analysis as evidenced by the absence of the 5′ (926B9) and 3′ (1002G3) PAC clone. Cytogenetic and FISH analysis showed a balanced translocation in a large NF1 family: t(14;17)(q32;q11.2), interrupting the NF1 gene as PAC 926B9 was found on the der (17) and PAC 1002G3 on the der (14) (data not shown).

32 of the mutations identified (including the translocation) are novel compared to the most recent data (International NF1 Mutation Analysis Consortium before, March 1999; URL address: http://www.nf.org/nflgene/nflgene.home.html), Ars et al, 2000, Fahsold et al., 2000 and most recent overview of published data by Upadhyaya and Cooper, 1998).

Translation inhibition facilitates detection of Premature Termination Codons (PTCs) by PTT and direct cycle sequencing

Nonsense mediated mRNA decay compromises most RNA-based mutation detection methods, but can be circumvented using puromycin (Andreutti-Zaugg et al., 1997). Starting from RNA extracted from Pmin EBV cultures, PTT detects truncated peptides even if mutant transcripts are highly unstable. However, direct cycle sequencing of cDNA fragments using fluorescent dyes is severely impaired by the nonsense mediated decay and the signal-to-noise ratio is far better starting from Pplus EBV cultures. Representative results are shown in FIG. 4B.

We compared the sensitivity of PTT and direct cycle sequencing in 13 EBV lymphoblastoid cell cultures treated with and without puromycin (6, FIG. 3, 4, 15 and 17). By PTT all truncated peptides from both types of cultures were discerned after 60 h exposure of autoradiograms. However only in 7 of the Pmin EBV cultures the mutant transcript was unambiguously identified by direct cDNA sequencing. In the remaining 6 cultures, the expression of the mutant transcripts was highly reduced compared to the normal transcripts with the ratio of mutant to wild-type peak height in sequencing chromatograms being <0.35. Direct cDNA cycle sequencing can not reveal unambiguously the pathogenic mutation in these cases. In contrast, in all Pplus EBV cultures the mutation was identified and the ratio between mutant and wild-type peak height in sequencing chromatograms varied between 0.8 and 1.00.

Distribution of the mutations, mutational hot spots and recurrent mutations

Mutations seem to be equally distributed along the gene. However, some exons may have a higher mutation density (FIG. 9A). In 15% of the patients studied, a mutation was found within the exons 10a-10b-10c, although this region comprises only 4.5% of the coding region. In this region 3 recurrent mutations (R440X, R461X and Y489C) were found. In E37, comprising only 1.2% of the coding region, a mutation was found in 5.9% of the patients. Ten recurrent mutations were identified in 20 unrelated patients and together account for 30% of the mutations found in this study (5): R304X, R440X, R461X, Y489C, 2033-2034insC, R1362X, R1513X, R1849Q, Y2264X and 7096-7101delAACTTT (FIG. 9B). As NF1 haplotypes were different in both families carrying the mutation R1849Q, recurrence can not be due to identity by descent.

R304X, R440X, R461X, R1362X, R1513X, R1849Q are C>T or G>A substitutions at CpG dinucleotides, which may explain their recurrence. There is no clue to explain the recurrence of 1466A>G (Y489C) looking at the sequences surrounding this mutational hotspot. We found this mutation in 5/232unrelated patients (Messiaen et al., 1999). The recurrence of 2033insC may be caused by slippage of the polymerase in a stretch of 7 cytosines. Mutation Y2264X (C6792A and C6792G) resides in a sequence environment containing direct AC-repeats as well as palindromic sequences. The recurrence of 7096delAACTTT may be caused by slipped mispairing between two AACTTT tandem repeat sequences.

Missense mutations in the NF1 gene and their pathogenicity

We identified 6 genuine missense mutations or deletions of single amino acids, i.e. C93Y, C187Y, L847P, 2970-2972delAAT and 7096-7101delAACTTT. These were absent on 300 normal control chromosomes, conserved during evolution in Rat (D45201), Mouse (L10370), Fugu (AF064564) and Drosophila (L26501), segregated with the disorder in 5 familial cases or verified to be de novo in 1 sporadic case. If the total coding region would be studied uniquely at the genomic level 4 more mutations could erroneously be considered to be missense mutations: Y489C (2X), V1093M (1X) and R2616Q (1X). Y489C was documented as a splice mutation (Messiaen et al., 1999). V1093M acts similarly as a splice mutation by creating a novel splice donor in the middle of E19b. R2616Q, found in a familial patient NF-027, was not found in 300 control normal chromosomes, is predicted to cause a dramatic change in the polypeptide chain and is conserved in Rat, Mouse, Fugu and Drosophila (FIG. 16). However, this alteration did not segregate with the disorder within the family (FIG. 16). The index patient was compound heterozygous for R2616Q and R304X, the latter identified by PTT. R304X is the genuine pathogenic mutation in this family as her healthy daughter inherited the R2616Q allele and the affected daughter the R304X mutation. This finding underscores the importance of the analysis of the total coding region for truncating mutations before firm conclusions can be made on the pathogenicity of missense mutations.

Mutations resulting in splicing defects

Splicing errors were detected in 19/67 (28%) patients (Table 5 and 7).

Only 4 splice mutations were at the canonical GT splice donor or AG splice acceptor, i.e. IVS12a+1G>T, 6577delGAGgta, IVS26-2A>T and IVS27b-2A>T.

6 mutations were at less conserved positions of the 5′ or 3′ splice site (ss): IVS16+3delaaagtg, R1849Q, K2286N, IVS16-6delcut, IVS19b-3C>G and IVS39-12T>A.

One nonsense and 2 missense mutations create a novel 5′ or 3′ ss and are splice mutations, i.e. S1765X, Y489C and V1093M.

Y2264X (C6792A and C6792G) result in E37 skipping and R304X results partially in E7 skipping besides retention of the nonsense codon (FIG. 5). Both mutations do not alter the existing normal ss nor create novel ones and may exert their effect by altered interaction between an exonic splice enhancer and mRNA splicing factors (Messiaen et al., 1997, Hoffmeyer et al., 1998). The remaining 25 nonsense mutations were not associated with splicing defects and hence nonsense mediated exon skipping is rather exceptional in NF1.

Consensus values (CVs) according to Shapiro and Senepathy (1987) and splice site scores (SSSs) according to Splice Site Prediction by Neural Networks (SSPNN) were calculated for all splice sites involved in splicing mutations (Table 7).

Simple skipping of an exon due to a mutation at the 5′ or 3′ consensus ss of that exon was observed in only 4 cases, i.e. 6577delGAGgta, K2286N, IVS16-6delcttt and IVS19b-3C>G. Only K2286N results in a transcript that, if translated, would leave the reading frame intact. Remaining splice mutations had complex effects.

IVS12a+1G>T leads to skipping of both E11 and 12a. Noteworthy, CV and SSS for the normal 5′ and 3′ ss of E11a and for the 3′ ss of E12a are very weak which may explain the concerted skipping of both exons if a mutation affects the 5′ ss of E12a. R1849Q (5547G>A) results in transcripts lacking E29 (ex29del) and transcripts lacking both E29+30 (ex29/30del) in equal amounts. The same transcripts were found in a patient with mutation IVS29+1G>C (Osborn et al., 1999). This region is involved in tissue-specific alternative splicing with ex29del expressed only in human brain tissues and ex29/30del detectable at low levels in all tissues (Park et al., 1998). Constitutive increase of the ex29del expression in non-brain tissues seems to be the pathogenic lesion causing NF1 in this family.

Seven mutations induced—beside exon skipping in some cases—the activation of a cryptic ss or the use of a novel created ss, i.e. IVS16+3delaaagtg, Y489C, V1093M, IVS26-2A>T (FIG. 10), IVS39-12T>A (FIG. 11) and S1765X. For all mutations affecting the 5′ ss of NF1 exons, the CV and SSS of the mutated ss (M) were lower than the wild type ss (N) sequences but differences between the SSSs were more pronounced.

Y489C (FIG. 12) and V1093M (FIG. 13) both create a novel 5′ ss with an almost identical CV compared to the wild type 5′ ss. Prediction of ss strength by SSPNN gives slightly better results (Table 7). The natural 5′ ss of E19b is unusual for primates (AA/gtaaat, rare nucleotides underlined), which may explain why it is inactivated even by the weak donor ss created by V1093M.

Mutations at the 3′ ss had a lower (⅗) or almost identical (⅖) CV and SSS compared to the wild type 3′ ss. Both the CVs or SSSs fail to predict whether the mutation will lead to exon skipping and/or activation of a cryptic 3′ ss.

IVS16-6delcttt and IVS39-12T>A both disrupt a tandem repeat, cttt and gttt respectively. For both mutations, the CV and SSS of the mutant sequence are identical compared to the wild type 3′ ss, yet missplicing occurs and the outcome of both mutations differs. IVS16-6delcttt leads to “simple” E17 skipping, although a strong cryptic 3′ ss resides 57 nt upstream (SSS 0.96) and—if activated—could result in the in frame insertion of 29 amino acids. For IVS39-12T>A the mutant 3′ sequence (gtttgttagttgtag/ggtacag (SEQ ID NO 15)) still has a high SSS (0.99) yet apparently gets inactivated and a novel created 3′ ss at IVS39-12 (gtttgtttgtttgtttgttag/tttttgtaggg) is used partially leading to a transcript that retains the last 10 nucleotides of IVS39, forming a peptide of 209 amino acids after in vitro translation. The mutation further causes skipping of E40 leading to a peptide shortened by only 44 amino acids. Both truncated peptides were discerned by PTT (FIG. 11) illustrating the power of this technique to detect multiple mutant transcripts.

The novel 3′ ss created by S1765X has a lower CV and SSS compared to the wild type ss, yet in frame skipping of the first 90 nucleotides of E29 is observed (FIG. 14). An intranuclear scanning mechanism capable of recognizing nonsense codons as proposed by Dietz and Kendzior (1994) and primarily concerned with the maintenance of an open reading frame may mediate this outcome.

R304X and splicing

R304X was shown by Hoffmeyer et al (1997) to result in in-frame E7 skipping without retention of the nonsense codon in the mutant transcripts. We studied 3 patients with mutation R304X from 2 unrelated families. Starting from RNA extracted from Pplus and Pmin EBV cultures, PTT clearly showed a truncated peptide of approximately 33 kDa, the size expected when the nonsense codon is retained in all patients (FIG. 15). Cycle sequencing of cDNA from the Pmin cultures showed unequal expression of the mutant transcript containing the nonsense codon (FIG. 15). Semi-quantitative RT-PCR analysis of the transcripts showed that E7 skipping was present in a minor fraction of the transcripts (6-13%; FIG. 5). Puromycin treatment did not alter the ratio of the E7-skipped versus full-length transcript (FIG. 5). In EBV lymphoblastoid cell lines, Ex7del transcripts were undetectable in normal control persons (data not shown) and in patients with the mutation Y2264X (FIG. 5). Our results indicate that at least in EBV cultures E7 skipping is not the major outcome of mutation R304X. We cannot exclude whether or not cell type dependent splicing differences may underly the apparent discrepancies between both studies.

Variant splicing due to environmental factors and not associated with mutations.

In specific tumor suppressor genes such as TSG101 and FHIT, some transcripts with internal deletions are not necessarily associated with a genomic mutation and can be found in the RNA from normal tissues as well, especially in lymphocytes not processed immediately after prelevation (“aged” blood) (Gayter et al., 1997). Initially PTT was developed starting from blood samples, but often spurious background bands were visible on autoradiograms, urging us to develop the technology starting from EBV transformed cell lines. Some blood samples are inevitably delayed in transit from the hospital to the laboratory and the background bands may be caused by misspliced NF1 transcripts in “aged” blood cells that lead to the formation of truncated peptides in the PTT. We analysed blood samples from 4 unrelated normal control persons for 2 regions in the NF1 transcript: the region where E7-skipping was observed to a low extent in patients with the mutation R304X and the region where equal expression of transcripts with E37-skipping was observed in patients with the mutation Y2264X (Messiaen et al., 1997). Semi-quantitative analysis showed that skipping of E7 and 37 was undetectable in all control samples processed immediately after prelevation of the blood. In all 4 “aged” blood samples however, a fraction of the transcripts showed skipping of E7 and E37. Typically the ratio of misspliced to full-length transcripts for E7 and E37 ranged between 0.09 and 0.14. Analyses starting from RNA that was not extracted immediately after prelevation of the blood samples can result in the occurrence of shorter transcripts in RT-PCR. This “noise” may in some cases obscure the real “signal” that is formed by the bona fide mutation.

Discussion

In this study 67 unrelated typical NF1 patients were analysed using a cascade of complemenatry techniques and the mutation was identified in 64 patients (>95%). Hence a sensitive molecular diagnostic test for NF1 can be achieved if classical NF1 patients are studied with multiple complementary and optimized techniques.

By PTT we identified mutations in 56/67 patients (83%). Missense mutations or small in frame insertions/deletions were found by HA in 6/67 patients (9%). 18 out of 44 single base pair substitutions were due to a C>T or G>A transition at CpG dinucleotides. By cytogenetic and/or FISH analysis 1 deletion of the entire gene and 1 balanced translocation t(14; 17)(q32;q11.2) interrupting the NF1 gene was found.

The mutations were evenly distributed along the NF1 coding sequence. However, exons 10a-10c and E37 seem to be more mutation-rich as would be expected if mutations were distributed at random. We did not find E4b to be a remarkably mutation-rich region, as indicated by Fahsold et al (in press). This may be due to our smaller patient cohort. Alternatively, some of the recurrent E4b mutations reported by Fahsold et al may be identical by descent as no data on sporadic versus familial status or haplotypes are available. If, for both studies, we consider the number of E4b mutations versus the number of patients that were studied instead of versus the number of mutations that were found, a similar pick-up rate for E4b is obtained (16 out of 521 patients by Fahsold et al and 2 out of 67 patients in this study; both ˜3%). Ten mutations were recurrent in our study and each account for ˜2.9% of the germline mutations. The high number of recurrent mutations was unexpected. Our results suggest that the exons 7, 10a-10c, 13, 23.2, 27a, 29, 37, 39 harbour recurrent mutations and that these exons, together with exons 4b, 22 and 31 reported by others to contain recurrent mutations (Fahsold et al., in press and Upadhyaya and Cooper, 1998), should be implemented with priority in NF1 mutation analysis.

A mutation was identified in 36 out of 39 sporadic patients (92%). This study shows for the first time that also in sporadic NF1 patients the pathogenic mutation can be identified with high efficiency. The most immediate result of this effort is the ability to provide presymptomatic/prenatal testing in the offspring of sporadic patients. Moreover, a sensitive test can help to diagnose young children presenting with NF1-related symptoms, but not (yet) fulfilling the N.I.H. diagnostic criteria.

The presence of somatic mosaicism in conditions with a high new mutation rate as in NF1 has been predicted (Hall, 1988). Comparisons between mutation detection rates after analyzing the total coding region in sporadic versus familial NF1 patients were not published so far. In our study the gDNA direct sequencing chromatograms of 2 patients suggest that the mutant and wild-type sequence are not present in equal amounts. This might reflect somatic mosaicism and is currently further investigated. All patients in whom we found no mutation are sporadic and low level somatic mosaicism may underly the failure to find a mutation. Alternatively, we may have missed the mutations as no technique is 100% sensitive or the mutations may reside in the exons 1 or 49 or in the 5′ or 3′ UTR that were not yet analysed.

In the majority of cases the clinical features of NF1 are caused by haploinsufficiency due to a mutation leading to a PTC and rapid decay of the mutant RNA. In this study 6 missense mutations and/or small in frame deletions were identified that may exert their effect in a dominant-negative fashion. Another group of mutations that may produce some truncated neurofibromin are mutations that affect splicing. The frequency of splicing errors in the NF1 gene is very high (28%) compared to other genetic disorders or as can be expected by calculation of relative target sizes (Krawczak et al., 1992). Only a minority of splice mutations (4/19) were found at the invariant AG/GT dinucleotides and mutations at 3′ ss were as frequent as at the 5′ ss. 8 splice mutations induce in frame skipping of total exons or part of an exon and have the potential to be “leaky”. As 4 of these splice mutations (S1765X, K2286N, C6792A and C6792G) form a stable mutant transcript it remains possible that a truncated neurofibromin is formed.

The observed splicing defects provide an unusual opportunity to examine splice site competition and the sequence determinants of splice site selection.

For some regions of the NF1 gene, we found exon-deleted transcripts in normal control persons. The presence of these transcripts was more pronounced in the RNA extracted from “aged” lymphocytes. Multiple alternatively spliced transcripts have been described for NF1 (Danglot et al., 1995; Suzuki et al., 1991; Cawthon et al., 1990; Park et al., 1998). The observation that other specific splice variants apparently are formed—albeit typically at low levels—if blood lymphocytes are not kept at physiological temperatures is intriguing. The results lend support to the hypothesis that epigenetic factors may contribute to the phenotypic variability in NF1 patients by altering the ratio of specific splice variants.

The fact that exon-deleted products are easily detected in PTT assays indicates that caution is needed in the interpretation of a “positive” PTT result, until a credible underlying mutation in the genomic DNA is identified. In particular, it is ill advised to use the results of the PTT for diagnostic purposes if the mutation at the cDNA and genomic DNA can not be identified.

Often clinical samples are delayed in transit and cDNA analysis of such samples may yield results that mimic splicing errors. We circumvented this problem by establishing EBV transformed cell cultures, however this is a time consuming and expensive step. Therefore, we are currently evaluating the efficiency and sensitivity of the PTT starting from short term cultures of phytohaemagglutinin stimulated lymphocytes. The combination of short term culture of stimulated lymphocytes and puromycin treatment may significantly decrease the time needed to identify in a reliable and sensitive way the mutations in the NF1 gene by PTT.

The availability of a powerful mutation detection technology for the NF1 gene will allow to adress some longstanding questions such as i/ what is the contribution of the NF1 gene to segmental NF, gastrointestinal NF, familial spinal NF, familial café-au-lait spots, late-onset NF and to conditions related to NF1 but with additional features; ii/ do genotype-phenotype correlations in NF1 exist; iii/ what is the contribution of somatic mosaicism in sporadic NF1 cases.

References

Abernathy C, Rasmussen S, Stalker H, Zori R, Driscoll D, Williams C, Kousseff B and Wallace M. 1997. NF1 mutation analysis using a combined heteroduplex/SSCP approach. Hum Mutat 9:548-554.

Andreutti-Zaugg C, Scott R and Iggo R. 1997. Inhibition of nonsense-mediated messenger RNA decay in clinical samples facilitates detection of human MSH2 mutations with an in vivo fusion protein assay and conventional techniques. Cancer Research 57:3288-3293.

Antonarakis S. 1998. Recommendations for a nomenclature system for human gene mutations. Nomenclature Working Group. Hum Mutat. 11:1-3.

Ars E, Serra E, Garcia J, Kruyer H, Gaona A, Lazaro C and Estivill X. 2000. Mutations affecting mRNA splicing are the most common molecular defects in patients with neurofibromatosis type 1. Hum Mol Genet 9:237-247.

Barker D, Wright E, Nguyen K, Cannon L, Fain P, Goldgar D, Bishop D, Carey J, Kivlin J, Willard H, Waye J S, Greig G, Leinwand L, Nakamura Y, O'Connell P, Leppert M, Lalouel J-M, White R, Skolnick H. 1987. Gene for von Recklinghausen neurofibromatosis is in the pericentromeric region of chromosome 17. Science 236:1100-1102.

Capsey, et al., 1988. Genetically Engineered Human Therapeutic Drugs, Stockton Press, New York.

Cawthon R, Weiss R, Xu G, Viskochil D, Culver M, Stevens J., Robertson M, Dunn D, Gesteland R, O'Connell P et al 1990. A major segment of the neurofibromatosis type 1 gene: cDNA sequence, genomic structure and point mutations. Cell 62:193-201.

Cichowski K, Shih T, Schmitt E, Santiago S, Reilly K, McLaughlin M, Bronson R and Jacks T. 1999. Mouse models of tumor development in neurofibromatosis type 1. Science 286:2172-2176.

Carey J. and Viskochil D. 1999. Neurofibromatosis Type 1: a model condition for the study of the molecular basis of variable expressivity in human disorders. Am J Med Genet (semin Med Genet) 89:7-13.

Claes K, Machackova E, Callens T, Van der Cruyssen G, Messiaen L. 1998. Improved conditions for PTT analysis of the BRCA1, BRCA2, NF1 and APC genes. Elsevier Trends Journals Technical Tips Online. t01457.

Cooper D, Krawczak M and Antonarakis S. 1998. The nature and mechanisms of human gene mutation. In: Vogelstein B and Kinzler K W, editors. The genetic basis of human cancer. McGraw-Hill p 65-94.

Correa C L, Brems H, Lazaro C, Estivill X, Clementi M, Mason S, Rutkowsky L, Marynen P, Legius E. 1999. Molecular studies in 20 submicroscopic neurofibromatosis type 1 gene deletions. Hum Mutat 14:387-393.

Cummings L, Glatfelter A. and Marchuk D. 1993. NF1-related loci on chromosomes 2, 12, 14, 15, 20, 21 and 22: a potential role of gene conversion in the high spontaneous mutation rate of NF1? Am. J. Hum. Genet 53: 672A.

Danglot G, Regnier V, Fauvet D, Vassal G, Kujas M, Bernheim A. 1995. Neurofibromatosis 1 (NF1) mRNAs expressed in the central nervous system are differentially spliced in the 5′ part of the gene. Hum Mol Genet 4:915-920.

Den Dunnen J and Van Ommen G-J. 1999.The protein truncation test: A review. Hum Mut 14:95-102.

Dietz H, Kendzior R. 1994. Maintenance of an open reading frame as an additional level of scrunity during splice site selection. Nat Genet 8:183-188.

Fahsold R, Hoffmeyer S, Mischung C, Gille C, Ehlers C, Kucukceylan N, Abdel-Nour M, Gewies A, Peters H, Kaufmann D, Buske A, Tinschert S and Nurnberg P. 2000. Minor lesion mutational spectrum of the entire NF1 gene does not explain its high mutability but points to a functional domain upstream of the GRD. Am J Hum Genet. 66, 790-818.

Gayter S, Barski P, Batley S, Li L, de Foy K, Cohen S, Ponder B and Caldas C. 1997. Aberrant splicing of the TSG 101 and FHIT genes occurs frequently in multiple malignancies and in normal tissues and mimics alterations previously described in tumours. Oncogene 15:2119-2126.

Gutmann D, Aylsworth A, Carey J, Korf B, Marks J and Pyeritz R., Rubenstein A and Viskochil D. 1997. The diagnostic evaluation and multidisciplinary management of neurofibromatosis 1 and neurofibromatosis 2. JAMA 278:51-57.

Hall J. 1988. Somatic mosaicism: observations related to clinical genetics. Am J Hum Genet 43:355-363.

Heim R, Kam-Morgan L, Binnie C, Corns D, Cayouette C , Farber R, Aylsworth A, Silverman L and Luce M. 1995. Distribution of 13 truncating mutations in the neurofibromatosis type 1 gene. Hum Mol Genet 4:975-981.

Hoffmeyer S, Assum G, Griesser J, Kaufmann D, Nurnberg P, Krone W. 1995) On unequal allelic expression of the neurofibromin gene in neurofibromatisis type 1 Hum Mol Genet 4:1267-1272.

Hoffmeyer S, Nurnberg P, Ritter H, Fahsold R, Leistner W, Kaufmann D, Krone W. 1998. Nearby stop codons in exons of the neurofibromatosis type 1 gene are disparate splice effectors. Am J Hum Genet 62:269-277.

Hulsebos T, Bijleveld E, Riegman P, Smink L and Dunham I. 1996. Identification and characterization of NF1-related loci on human chromosomes 22, 14 and 2. Hum Genet 98:7-11.

Huson S and Hughes R .1994.The neurofibromatoses: a clinical and pathogenetic overview. Chapman and Hall, London.

Kayes L, Riccardi V, Burke W, Bennet R. and Stephens K. 1992. Sporadic neurofibromatosis 1, mental retardation and dysmorphism. J Med Genet 29:686-687.

Kim M. R. and Tamanoi F. 1998 “Neurofibromatosis 1 GTPase activating protein-related domain and its functional significance” In the book: Neurofibromatosis Type 1, from genotype to phenotype, Eds. M. Upadhyaya and D. Cooper, Bios Scientific Publishers.

Krawczak M, Reiss J and Cooper D. 1992. The mutational spectrum of single base-pair substitutions in mRNA splice junctions of human genes: causes and consequences. Hum Genet 90:41-54.

Lambert J, Naeyaert J M, Callens T, De Paepe A and Messiaen L. 1998. Human myosin V gene produces different transcripts in a cell type-specific manner. Biochem Biophys Res Commun 252:329-33.

Lambert J, Naeyaert J. M., De Paepe A, Van Coster R, Ferster A, Song M, and Messiaen L. 2000. Arg-Cys substitution at codon 1246 of the human myosin Va gene is not associated with Griscelli syndrome. J. Invest. Dermatology 114:731-733.

Li Y, O'Connell P, Breidenbach H H, Cawthon R, Stevens J, Xu G, Neil S, Robertson M, White R, Viskochil D. 1995. Genomic organization of the neurofibromatosis 1 gene (NF1). Genomics 25:9-18.

Marchuk D, Saulino A, Tavakkol R, Swaroop M, Wallace M, Anderson L, Mitchell A, Gutmann D, Bogushi M and Collins F. 1991. cDNA cloning of the type 1 neurofibromatosis gene: complete sequence of the NF1 gene product. Genomics 11:931-940.

Mathew C, Thorpe K, Easton D, Chin K, Jadayel D, Ponder M, Moore G, Wallis C, Slater C, De Jong G et al., 1989. Linkage analysis of chromosome 17 markers in British and South African families with neurofibromatosis type 1. Am J Hum Genet 44:38-40.

Maynard J, Krawczak M, Upadhyaya M. 1997. Characterisation and significance of nine novel mutations in exon 16 of the neurofibromatosis type 1 (NF1) gene. Hum-Genet 99:674-6.

Messiaen L, Callens T, De Paepe A, Craen M and Mortier G. 1997. Characterization of two different nonsense mutations, C6792A and C6792G, causing skipping of exon 37 in the NF1 gene. Hum Genet 101:75-80.

Messiaen L, Callens T, Roux K, Mortier G, De Paepe A, Abramowicz M, Pericak-Vance M, Vance J, and Wallace M. 1999. Exon 10b of the NF1 gene represents a mutational hotspot and harbers a recurrent missense mutation Y489C associated with aberrant splicing. Genetics in Medicine 1: 248-253.

Messiaen L., Callens T., Mortier G., Beysen D., Vandenbroucke I., Van Roy N., Speleman F. and De Paepe A. 2000. Exhaustive mutation analysis of the NF1 gene allows to identify 95% of the mutations and reveals a high frequency of unusual splicing defects. Hum Mutat. 15:541-555, 2000 (in press).

Osborn M, Upadhyaya M. 1999. Evaluation of the protein truncation test and mutation detection in the NF1 gene: mutational analysis of 15 known and 40 unknown mutations. Hum Genet 105:327-332.

Park V and Pivnick. E. 1998. Neurofibromaosis type 1 (NF): a protein truncation assay yielding identification of mutations in 73% of patients. J Med Genet 35:813-820.

Park V, Kenwright K, Sturtevant D, Pivnick E. 1998. Alternative splicing of exons 29 and 30 in the neurofibromatosis type 1 gene. Hum Genet 103:382-385.

Purandare S, Lanyon W. and Connor J. 1994.Characterisation of inherited and sporadic mutations in neurofibromatosis type-1. Hum Mol Genet 3:1109-1115.

Rasmussen S and Friedman J. 2000. NF1 gene and neurofibromatosis 1. Am J. Epidemiol. 151:33-40.

Riccardi V. 1993. Genotype, malleotype, phenotype and randomness: lessons from neurofibromatosis I (NF1). Am J Hum Genet 53: 301-304.

Roest P, Roberts R, Sugino S, Van Ommen G-J and Den Dunnen. 1993. J Protein truncation test (PTT) for rapid detection of translation-terminating mutations. Hum Mol Genet 2: 1719-1721.

Ruttledge M, Andermann A, Phelan C, Claudio J, Han F, Chretien N, Rangaratnam S, MacCollin M, Short P, Parry D, Michels V, Riccardi V, Weksberg R, Kitamura K, Bradburn J, Hall B, Propping P. and Rouleau G. 1996. Type of mutation in the neurofibromatosis type 2 gene (NF2) frequently determines severity of disease. Am. J. Hum Genet. 59: 331-342.

Sambrook J, Fritsch E. F, Maniatis T. 1989. Molecular Cloning: A Laboratory Manual. Cold Spring Harbor Press, Cold Spring Harbor, N.Y.

Shapiro M, Senepathy P. 1987. RNA splice junctions of different classes of eukaryotes: sequence statistics and functional implications in gene expression. Nucl Acids Res 15:7155-7174.

Shen M, Harper P, Upadhyaya M. 1993. Neurofibromatosis type 1 (NF1): the search for mutations by PCR-heteroduplex analysis on Hydrolink gels. Hum Mol Genet 2:1861-1864.

Stumpf D, Alksne J, Annegers J, Brown S, Conneally P, Housman D and Leppert M. 1988.Neurofibromatosis conference statement. Arch Neurol 45:575-578.

Suzuki Y, Suzuki H, Kayama T, Yoshimoto T, Shibahara S. 1991. Brain tumors predominantly express the neurofibromatosis type 1 gene transcripts containing the 63 base insert in the region coding for GTPase activating protein-related domain. Biochem Biophys Res Commun 181:955-961.

Teraoka S, Telatar M, Becker-Catania S, Liang T, Onengut S, Tolun A, Chessa L, Sanal O, Bernatowska E, Gatti R, and Concannon P. 1999. Splicing defects in the ataxia-telangiectasia gene, ATM: underlying mutations and consequences. Am. J. Hum. Genet 64: 1617-1631.

Toliat M, Erdogan F, Gewies A, Fahsold R, Buske A, Tinschert S, Nurnberg P. 2000. Analysis of the NF1 gene by temperature gradient gel electrophoresis reveals a high incidence of mutations in exon 4b. Electrophoresis 21: 541-544.

Upadhyaya M and Cooper D, 1998. Eds. Neurofibromatosis type 1 from genotype to phenotype, BIOS Scientific Publishers.

Upadhyaya M, Cooper D. 1998. The mutational spectrum in neurofibromatosis 1 and its underlying mechanisms. In: Upadhyaya M and Cooper D, editors. Neurofibromatosis type 1 from genotype to phenotype. Oxford: BIOS Scientific Publishers p65-88.

Van Roy N., Laureys G., Cheng N. C., Willem P., Opdenakker G., Versteeg R., Speleman F. 1994. 1;17 translocations and other chromosome 17 rearrangements in human primary neuroblastoma tumours and cell lines. Genes, Chromosomes & Cancer 10:103-114 (1994).

Viskochil D. 1998. “Gene structure and expression” In: “NF1: from genotype to genotype” Eds. Upadhyaya and Cooper, p 39-56.

Viskochil D, Buchberg A, Xu G, Cawthon R, Stevens J. et al 1990. Deletion and a translocation interrupt a cloned gene at the neurofibromatosis type 1 locus. Cell 62:187-192.

Vogel K, Klesse L, Velasco-Miguel S, Meyers K, Rushing E and Parada L 1999. Mouse tumor model for neurofibromatosis type 1. Science 286: 2176-2179.

Wallace M, Marchuk D, Anderson L, Letcher R, Odeh H., Saulino A. et al 1990. Type 1 neurofibromatosis gene: identification of a large transcript disrupted in three NF1 patients. Science 249:182-186.

Wallace M, Trimpert R, Thomson A, Fishbein L. and Abernathy C. 1998. Cryptic out-of-frame NF1 exon splicing in neurofibromas Am J Hum Genet 63 Supp:A90.

Wu B, Schneider G and Korf B. 1997. Deletion of the entire NF1 gene causing distinct manifestations in a family. Am J Hum Genet 69:98-101.

Wu B, Austin M, Schneider G, Boles R and Korf B. 1995. Deletion of he entire NF1 gene detected by FISH: four deletion patients associated with severe manifestations. Am J Med Genet 59:528-535.

TABLE 1
Mutations detected by analysis of the whole coding region of the NF1 gene by
PTT, HA, Cytogenetic and FISH analysis.

| | Genomic | | Codon | Codon | Exon/ |
|---|---|---|---|---|---|
| Patient | Mutation¹ | Effect on cDNA | Change | Number | Intron |
| NF-018 | t(14; 17)(q32; q11.2) | / | / | / | / |
| NF-096 | Del total gene | / | / | / | / |
| NF-097 | Del total gene | / | / | / | / |
| NF-098 | Del total gene | / | / | / | / |
| NF-062 | Del total gene | / | / | / | / |
| NF-077 | 99A > G | 99del105 | K > K | 33 | 2 |
| NF-004 | 247C > T | 247C > T | Q > X | 83 | 3 |
| NF-023 | 278G > A | 278G > A | C > Y | 93 | 3 |
| NF-036 | 560G > A | 560G > A | C > Y | 187 | 4b |
| NF-080 | 574C > T | 574C > T | R > X | 192 | 4b |
| NF-056 | 574C > T | 574C > T | R > X | 192 | 4b |
| NF-029 | 603-604insT | 603-604insT | frameshift | 202 | 4c |
| NF-090 | 625C > T | 625C > T | Q > X | 209 | 4c |
| NF-086 | 819-821delCCT | 819-821delCCT | delL | 274 | 6 |
| NF-088 | 889-454del474 | 888del174 | Del58aa | / | IVS6 en E7 |
| NF-027 | 910C > T | 910C > T; | R > X | 304 | 7 |
| | | 888del174 | | | |
| NF-064 | 910C > T | 910C > T; | R > X | 304 | 7 |
| | | 888del174 | | | |
| NF-081 | 910C > T | 910C > T; | R > X | 304 | 7 |
| | | 888del174 | | | |
| NF-024 | 987-988insA | 987-988insA | frameshift | 330 | 7 |
| NF-066 | 1019delCT | 1019delCT | Frameshift | | 7 |
| NF-093 | 1261 − 19G > A | 1260insTTGTTTTTCTCTAGC | Frameshift | SEQ ID NO 16 | IVS9 |
| NF-048 | 1275G > A | 1275G > A | W > X | 425 | 10a |
| NF-042 | 1318C > T | 1318C > T | R > X | 440 | 10a |
| NF-057 | 1318C > T | 1318C > T | R > X | 440 | 10a |
| NF-030 | 1381C > T | 1381C > T | R > X | 461 | 10a |
| NF-026 | 1381C > T | 1381C > T | R > X | 461 | 10a |
| NF-017 | 1466A > G | 1465del62 | Y > C | 489 | 10b |
| NF-012 | 1466A > G | 1465del62 | Y > C | 489 | 10b |
| NF-065 | 1466A > G | 1465del62 | Y > C | 489 | 10b |
| NF-095 | 1466A > G | 1465del62 | Y > C | 489 | 10b |
| NF-016 | 1465-1466insC | 1466insC | Y > X | 489 | 10b |
| NF-068 | 1527 + 5G > A | 1392del135 | NA | NA | IVS10b |
| NF-033 | 1570G > T | 1570G > T | E > X | 524 | 10c |
| NF-073 | 1605insA | 1605insA | | | 10c |
| NF-052 | 1607C > A | 1607C > A | S > X | 536 | 10c |
| NF-076 | 1642 − 3C > G | 1641del80 | frameshift | | IVS10c |
| NF-045 | IVS12a + 1G > T | 1641del204 | none | NA | IVS12a |
| NF-034 | 2033-2034insC | 2033insC | frameshift | 678 | 13 |
| NF-035 | 2033-2034insC | 2033insC | frameshift | 678 | 13 |
| NF-074 | 2305insT | 2305insT | frameshift | | 14 |
| NF-010 | 2540T > C | 2540T > C | L > P | 847 | 16 |
| NF-070 | 2585insA | 2585insA | frameshift | | 16 |
| NF-071 | 2836insT | 2836insT | frameshift | | 16 |
| NF-051 | IVS16 + 2del6 | 2617del233 | frameshift | NA | IVS16 |
| NF-028 | IVS16-6del4 | 2850del140 | frameshift | NA | IVS16 |
| NF-014 | 2875C > T | 2875C > T | Q > X | 959 | 17 |
| NF-011 | 2887C > T | 2887C > T | Q > X | 963 | 17 |
| NF-001 | 2970-2972delAAT | 2970-2972delAAT | delM | 991 | 17 |
| NF-091 | 2990 + 3A > C | 2850del140 | Frameshift | NA | IVS17 |
| NF-072 | 3132C > A | 3132C > A | Y > X | 1044 | 19a |
| NF-053 | 3194delC | 3194delC | frameshift | 1065 | 19a |
| NF-063 | 3277G > A | 3277G > A, 3274del40 | V > M; frameshift | 1093 | 19b |
| NF-025 | IVS19b − 3C > G | 3314del182 | frameshift | NA | IVS19b |
| NF-039 | 3367G > T | 3367G > T | E > X | 1123 | 20 |
| NF-089 | 3457delCTCA | 3457delCTCA | Frameshift | NA | 20 |
| NF-046 | 3520C > T | 3520C > T | Q > X | 1174 | 21 |
| NF-083 | 3704delA | 3704delA | Frameshift | NA | 21 |
| NF-085 | 3708 + 1G > C | 3496del212 | frameshift | NA | IVS21 |
| NF-078 | 3826C > T | 3826C > T | R > X | 1276 | 22 |
| NF-059 | 3826C > T | 3826C > T | R > X | 1276 | 22 |
| NF-094 | 3826C > T | 3826C > T | R > X | 1276 | 22 |
| NF-079 | 4026delG | 4026delG | frameshift | NA | 23.2 |
| NF-041 | 4084C > T | 4084C > T | R > X | 1362 | 23.2 |
| NF-054 | 4084C > T | 4084C > T | R > X | 1362 | 23.2 |
| NF-082 | 4299delC | 4299delC | Frameshift | NA | 25 |
| NF-067 | 4480C > T | C > T | Q > X | 1494 | 26 |
| NF-044 | IVS26 − 2A > T | 4515-14 ins14; | frameshift | NA | IVS26 |
| | | 4515-17ins17 | | | |
| NF-021 | 4537C > T | 4537C > T | R > X | 1513 | 27a |
| NF-006 | 4537C > T | 4537C > T | R > X | 1513 | 27a |
| NF-058 | IVS27b − 2A > T | 4772del433; 4772del293 | frameshift | NA | IVS27b |
| NF-049 | 5033delG | 5033delG | frameshift | 1678 | 28 |
| NF-084 | 5717delT | 5117delT | Frameshift | | 30 |
| NF-009 | 5264C > G | 5264C > G | S > X | 1755 | 29 |
| NF-050 | 5294C > A | 5215del90 (5294C > A???) | S > X | 1765 | 29 |
| NF-037 | 5546G > A | 5205del341; 5205del544 | R > Q | 1849 | 29 |
| NF-038 | 5546G > A | 5205del341; 5205del544 | R > Q | 1849 | 29 |
| NF-069 | 5546 + 2T > G | 5205del341; 5205del544 | frameshift | NA | 29 |
| NF-047 | 5567delT | 5567delT | frameshift | 1856 | 30 |
| NF-031 | 5798delC | 5798delC | frameshift | 1933 | 31 |
| NF-060 | 5839C > T | 5839C > T | R > X | 1947 | 31 |
| NF-043 | 5896C > T | 5896C > T | Q > X | 1966 | 31 |
| NF-015 | 6577delGAGgta | 6364del215 | frameshift | 2193 | 34 |
| NF-040 | 6709C > T | 6709C > T | R > X | 2237 | 36 |
| NF-008 | 6789-6792delTTAC | 6789-6792delTTAC | frameshift | 2263 | 37 |
| NF-019 | 6792C > A | 6756del102 | Y > X | 2264 | 37 |
| NF-003 | 6792C > G | 6756del102 | Y > X | 2264 | 37 |
| NF-032 | 6858G > C | 6756del102 | K > N | 2286 | 37 |
| NF-013 | 7096-7101del6 | 7096-7101del6 | delNF | 2366 | 39 |
| NF-022 | 7096-7101del6 | 7096-7101del6 | delNF | 2366 | 39 |
| NF-005 | IVS39 − 12T > A | 7126del132; | NA | NA | IVS39 |
| | | 7127-10ins 10 | | | |
| NF-002 | 7201A > T | 7201A > T | K > X | 2401 | 40 |
| NF-092 | 7268delCA | 7268delCA | Frameshift | 2423 | 41 |
| NF-055 | 7285C > T | 7285C > T | R > X | 2429 | 41 |
| NF-075 | 7285C > T | 7285C > T | R > X | 2429 | 41 |
| NF-061 | 7486C > T | 7286C > T | R > X | 2496 | 42 |
| NF-020 | 7884-7885delGT | 7884-7885delGT | frameshift | 2628 | 45 |
| NF-007 | 8016delA | 8016delA | frameshift | 2672 | 46 |

| Patient | Type/effect | PTT (frag)² | CG³ | S/F⁴ | Previously described |
|---|---|---|---|---|---|
| NF-018 | / | / | / | F | Novel |
| NF-096 | / | / | / | S | / |
| NF-097 | / | / | / | S | / |
| NF-098 | / | / | / | F | / |
| NF-062 | / | / | / | S | / |
| NF-077 | Splice: creation of novel 5'ss, IF skip 105 nt | +(F1) | no | | Novel |
| NF-004 | nonsense | +(F1) | no | F | Osborn et al., 1999 |
| NF-023 | missense | −(HA) | no | F | Novel |
| NF-036 | missense | −(HA) | no | F | Novel |
| NF-080 | nonsense | +(F1) | Yes | | Fahsold et al, 2000 |
| NF-056 | nonsense | +(F1) | yes | S | Fahsold et al., 2000 |
| NF-029 | frameshift | +(F1) | NA | F | Novel |
| NF-090 | nonsense | +(F1) | | S | Novel |
| NF-086 | Amino acid deletion | — | | | Novel |
| NF-088 | Genomic microdeletion of 474 basepairs | +(F1) | NA | S | Novel |
| NF-027 | nonsense: truncation due to stopcodon; | +(F1) | yes | S | Hoffmeyer et al., 1998 |
| | splice: intact 3' and 5'ss, no cryptic or novel ss, IF | | | | |
| | skip E7 | | | | |
| NF-064 | identical as NF-027 | +(F1) | yes | F | Hoffmeyer et al., 1998 |
| NF-081 | identical as NF-027 | +(F1) | yes | | Hoffmeyer et al., 1998 |
| NF-024 | frameshift | +(F1) | NA | F | Novel |
| NF-066 | Frameshift | +(F1) | NA | fnyi | Fahsold et al., 2000 |
| NF-093 | frameshift | +(H1) | NA | F | Novel |
| NF-048 | nonsense | +(F1) | no | S | Novel |
| NF-042 | nonsense | +(F1) | yes | S | Heim et al., 1995 |
| NF-057 | nonsense | +(F1) | yes | S | Heim et al., 1995 |
| NF-030 | nonsense | +(F1) | yes | S | Fahsold et al., in press |
| NF-026 | nonsense | +(F1) | yes | S | Fahsold et al., in press |
| NF-017 | splice: intact WT 5'ss, creation novel 5'ss, | +(F1) | no | F | Messiaen et al., 1999 |
| | skip last 62 nt E10b forms immediate | | | | |
| | stopcodon | | | | |
| NF-012 | identical as NF-017 | +(F1) | no | S | Messiaen et al., 1999 |
| NF-065 | identical as NF-017 | +(F1) | no | S | Messiaen et al., 1999 |
| NF-095 | identical as NF-017 | +(F1) | No | S | Messiaen et al., 1999 |
| NF-016 | nonsense | +(F1) | NA | F | Novel |
| NF-068 | Splice: inactiv 5'ss, IF skip E10b | +(F1) | no | F | Novel |
| NF-033 | nonsense | +(F1) | no | S | Novel |
| NF-073 | frameshift | +(F1) | NA | S | Novel (Wallace/Messiaen) |
| NF-052 | nonsense | +(F1) | no | F | Novel |
| NF-076 | Splice: inact 3'ss, OOF skippinf E11 | +(F1 + 2) | | | Novel |
| NF-045 | splice: inactiv 5'ss, IF skip E11 + 12a | +(F2) | no | S | Abernathy et al., 1997 |
| NF-034 | frameshift | +(F2) | NA | S | Heim et al., 1995 |
| NF-035 | frameshift | +(F2) | NA | S | Heim et al., 1995 |
| NF-074 | frameshift | +(F2) | NA | | Novel |
| NF-010 | missense | −(HA) | no | F | Messiaen et al., 1998 |
| NF-070 | frameshift | +(F2) | NA | F | Novel |
| NF-071 | frameshift | +(F2) | NA | fnyi | Novel |
| NF-051 | splice: inact 5'ss, activ cryptic 5'ss, skip last | +(F2) | NA | S | Novel |
| | 233 nt E16 | | | | |
| NF-028 | splice: inactiv 3'ss, OOF skip E17 | +(F2) | NA | F | Novel |
| NF-014 | nonsense | +(F2) | no | F | Novel |
| NF-011 | nonsense | +(F2) | no | F | Novel |
| NF-001 | amino acid deletion | −(HA) | NA | F | Shen et al., 1993 |
| NF-091 | Splice: inactiv 5'ss, OOF skip E17 | +(F2) | | S | Novel |
| NF-072 | nonsense | +(F2) | no | fnyi | Novel |
| NF-053 | frameshift | +(F2) | NA | S | Novel |
| NF-063 | splice: intact WT 5'ss, creation novel 5'ss, | +(F2) | no | F | Novel |
| | skip last 40 nt E19b | | | | |
| NF-025 | splice: inactiv 3'ss, OOF skip E20 | +(F2) | no | F | Novel |
| NF-039 | nonsense | +(F2) | no | S | Novel |
| NF-089 | Frameshift | +(F2 + F3) | No | F | Novel |
| NF-046 | nonsense | +(F2) | no | S | Novel: MOSAIC!! |
| NF-083 | Frameshift | +(F3) | | | Novel |
| NF-085 | frameshift | +(F3) | | | Novel |
| NF-078 | nonsense | +(F2) | Yes | | Heim et al., 1995 |
| NF-059 | nonsense | +(F3) | yes | S | Heim et al., 1995 |
| NF-094 | Nonsense | +(F3) | Yes | S | Heim et al., 2000 |
| NF-079 | frameshift | +(F3) | | | Novel |
| NF-041 | nonsense | +(F3) | yes | S | Upadhyaya et al., 1997 |
| NF-054 | nonsense | +(F3) | yes | S | Upadhyaya et al., 1997 |
| NF-082 | Frameshift | +(F3) | | | Novel |
| NF-067 | nonsense | +(F3) | no | fnyi | Novel |
| NF-044 | splice: inactiv 3'ss, activ 2 cryptic 3'ss sites | +(F3) | no | S | Novel |
| | leading to 2 different OOF insertions | | | | |
| NF-021 | nonsense | +(F3) | yes | S | Side et al., 1995 |
| NF-006 | nonsense | +(F3) | yes | S | Side et al., 1995 |
| NF-058 | splice: inactv 3'ss, activ cryptic 3'ss, OOF | +(F3) | no | F | Novel |
| | skip E28; OOF skip first 293 nt E28 | | | | |
| NF-049 | frameshift | +(F3) | NA | F | Novel |
| NF-084 | frameshift | +(F4) | | | Novel |
| NF-009 | nonsense | +(F4) | no | S | Novel |
| NF-050 | splice: intact 3'ss, creation novel 3'ss, IF | +(F4) | yes | F | Novel |
| | skip first 90 nt E29 | | | | |
| NF-037 | splice: inact 5'ss, OOF skip E29 and E29 + 30 | +(F4) | yes | F* | Fahsold et al., in press |
| NF-038 | identical as NF-037 | +(F4) | yes | F* | Fahsold et al., in press |
| NF-069 | identical as NF-037 | +(F4) | yes | S | Fahsold et al., in press |
| NF-047 | frameshift | +(F4) | NA | S | Novel |
| NF-031 | frameshift | +(F4) | NA | S | Novel |
| NF-060 | nonsense | +(F4) | yes | F | Cawthon et al., 1990 |
| NF-043 | nonsense | +(F4) | no | F | novel |
| NF-015 | splice: inactiv 5'ss, OOF skip E34 | +(F4) | NA | S | novel |
| NF-040 | nonsense | +(F4) | yes | S | Fahsold et al., in press |
| NF-008 | frameshift | +(F4) | NA | S | Robinson et al., 1995 |
| NF-019 | splice: intact 3'and 5'ss, no cryptic or novel | +(F4) | no | F | Messiaen et al., 1997 |
| | ss, IF skip E37 | | | | |
| NF-003 | identical as NF-019 | +(F4) | no | F | Messiaen etal, 1997 |
| NF-032 | splice: inactiv 5'ss, IF skip E37 | +(F4) | no | F | novel |
| NF-013 | amino acid deletion | −(HA) | NA | S | Abernathy et al., 1994 |
| NF-022 | amino acid deletion | −(HA) | NA | F | Abernathy et al,. 1994 |
| NF-005 | splice: inactiv 3'ss, IF skip E40; activ cryptic | +(F5) | no | F | novel |
| | 3'ss, OOF ins last 10 nt IVS39 | | | | |
| NF-002 | nonsense | +(F5) | no | S | novel |
| NF-092 | Frameshift | +(F5) | No | S | novel |
| NF-055 | nonsense | +(F5) | yes | S | Fahsold et al., in press |
| NF-075 | nonsense | +(F5) | yes | S | Fahsold et al., in press, low |
| | | | | | level MOSAIC in the |
| | | | | | lymphocytes!! |
| NF-061 | nonsense | +(F5) | yes | S | Purandare et al., 1994 |
| NF-020 | frameshift | +(F5) | NA | S | novel |
| NF-007 | frameshift | +(F5) | NA | S | novel |

¹Numbering based on NF1 cDNA (Genbank M82814).
²+ denotes that a truncated peptide was seen; - denotes that no truncated peptide was seen but mutation was identified by heteroduplex analysis (HA); (F1 to F5) denotes that the truncated peptide was seen after in vitro translation of RT-PCR fragments F1 to F5 using primers for amplifying NF1 cDNA as described by Heim et al (1995). F1 spans cDNA nt 1-1868, F2 1468-583, F3 3217-5256, F4 4998-6987 and F5 6574-404.
³For all 44 transitions is mentioned whether they change aG > A or C > T at a CpG dinucleotide.
⁴Sporadic (S) or familial (F) patient.
Abbrebiations: E exon/IVS intervening sequence or intron; IF, inframe; OOF, out of frame; ss splice site; nt nucleotide; skip skipping; inactiv. inactivation; activ. activation; NAnot applicable, fnyi: family not yet investigated (novel families: cfr red);
*NF-037 and NF-038 are proven to be unrelated as the mutation resides on a different haplotype background in both families.

TABLE 2
NF1 PRIMERS SEQUENCING c-DNA

| Name + Label | AnnT° | Position | Sequence (5′-3′) | Origin |
|---|---|---|---|---|
| NF.1cy | 60 | 1 | atggccgcgcacaggccggt | own |
| | | | (SEQ ID NO 16) | |
| NF1s1 cy | 58 | 73 | acaggacagcagaacaca | Rina Wu |
| | | | (SEQ ID NO 17) | |
| NF1s2 cy | 58 | 400 | cttcggaattctgcctct | Rina Wu |
| | | | (SEQ ID NO 18) | |
| NF1s3 cy | 58 | 713 | ctgatatggctgaatgtg | Rina Wu |
| | | | (SEQ ID NO 19) | |
| NF2s1 fl, cy | 58 | 967 | gcctgtgtcaaactgtgt | Rina Wu |
| | | | (SEQ ID NO 20) | |
| NF2s2 fl, cy | 58 | 1367 | cacacccagcaatacgaa | Rina Wu |
| | | | (SEQ ID NO 21) | |
| NF2s3, on, fl, cy | 58 | 1685 | atcctgatgctcctgtag | Rina Wu |
| | | | (SEQ ID NO 22) | |
| NF2s3b cy | 55 | 1878 | tttttacggggtaggatg | own |
| | | | (SEQ ID NO 23) | |
| NF3s1, on, fl, cy | 60 | 2035 | atttgccgacaagcccag | Rina Wu |
| | | | (SEQ ID NO 24) | |
| NF3s2, on, fl, cy | 58 | 2308 | actgcaggaaacactgag | Rina Wu |
| | | | (SEQ ID NO 25) | |
| NF3s3, on, fl, cy | 58 | 2689 | ggctgttgtccttaatgg | Rina Wu |
| | | | (SEQ ID NO 26) | |
| NF4s1, on, fl, cy | 58 | 3001 | tgcttgggaatatggtc | Rina Wu |
| | | | (SEQ ID NO 27) | |
| NF4s1a, cy(H3b) | 58 | 3229 | gtttcacttctagctggtct | own |
| | | | (SEQ ID NO 28) | |
| NF4s1b, on, cy | 60 | 3376 | caaacaggtggcaggaaacg | own |
| | | | (SEQ ID NO 29) | |
| NF4s2 fl, cy | 56 | 3558 | ccttcaacaaggcacaga | Rina Wu |
| | | | (SEQ ID NO 30) | |
| NF4s3 fl, cy | 56 | 3756 | actctaccaactgctctg | Rina Wu |
| | | | (SEQ ID NO 31) | |
| NF5s1 fl, cy | 55 | 4011 | gaacctccttcagatgac | Rina Wu |
| | | | (SEQ ID NO 32) | |
| NF5s3 fl, cy | 55 | 4308 | agaagaacatatgcggcc | Rina Wu |
| | | | (SEQ ID NO 33) | |
| NF5s3b rev cy | 58 | 4450 | gcacattgccgtcacttatg | own |
| | | | (SEQ ID NO 34) | |
| NF5s4 fl, cy | 55 | 4658 | ctaggcatcaggtacatg | Rina Wu |
| | | | (SEQ ID NO 35) | |
| NF6s1 fl, cy | 55 | 4957 | gtctccgcagtctatatc | Rina Wu |
| | | | (SEQ ID NO 36) | |
| NF6s1b cy | 60 | 5071 | cctgggaaactggctgagca | own |
| | | | (SEQ ID NO 37) | |
| NF6s2 fl, cy | 55 | 5382 | ggagtgtgaagccattgt | Rina Wu |
| | | | (SEQ ID NO 38) | |
| NF6s3 fl, cy | 55 | 5670 | tagtaagacgctggcagc | Rina Wu |
| | | | (SEQ ID NO 39) | |
| NF6s4 fl, cy | 55 | 5934 | ccttgggcagattacaga | Rina Wu |
| | | | (SEQ ID NO 40) | |
| NF7s1 fl, cy | 55 | 6195 | gatgctgtccttcaacaa | Rina Wu |
| | | | (SEQ ID NO 41) | |
| NF7s2 fl, cy | 52 | 6544 | gaaacagtcacagaagct | Rina Wu |
| | | | (SEQ ID NO 42) | |
| NF7s2b, cy | 57 | 6576 | ggaggcatgcatgagagata | own |
| | | | (SEQ ID NO 43) | |
| NF7s3 fl, cy | 54 | 6841 | cagccacttcttaataagg | Rina Wu |
| | | | (SEQ ID NO 44) | |
| NF7s4 fl, cy | 58 | 7134 | gcatccttcacctgctatt | Rina Wu |
| | | | (SEQ ID NO 45) | |
| NF8s1 fl, cy | 55 | 7375 | catggtgacccttcctat | Rina Wu |
| | | | (SEQ ID NO 46) | |
| NF8s2 fl, cy | 52 | 7742 | atgttctcttggatgaag | Rina Wu |
| | | | (SEQ ID NO 47) | |
| NF8s3 fl, cy | 50 | 8119 | gctgagcttattgttaag | Rina Wu |
| | | | (SEQ ID NO 48) | |

TABLE 3

NF1
Primers g-DNA: Primers for amplification of all exons of the NF1 gene for HA

| | | | | | |
|---|---|---|---|---|---|
| Name + la- | | | | | |
| bel | Program | PCR | Length | Sequence (5′-3′) | Sequence origin |

| | | | | | | |
|---|---|---|---|---|---|---|
| NFex1.1cy | does not work | | | | cy-cccagcctccttgccaacgc | Shen et al |
| | | | | | (SEQ ID NO 49) | |
| NFex1.2 | | | | | gacccattccaccggcctgt | |
| | | | | | (SEQ ID NO 50) | |
| NFex2x.1 | 95° 5′ | 100 ng | 340 | bp | tttcaatggcaagtaagt | own |
| | | | | | (SEQ ID NO 51) | |
| NFex2x.2 | (95°45″-54°30″- | 10 × CS | | | gttatatccaaagtccaca | own |
| | 72°30″) × 35 | | | | (SEQ ID NO 52) | |
| NFex2x.1cy | 72°10′ | 1UPltaq | | | | |
| NFex2.1fl | 4° | 25 μl | | | fluo-tttaaggataaactgtt | own |
| (nested) | | | | | (SEQ ID NO 53) | |
| NFex3.1 | HS | 100 ng | | | tttcacttttcagatgtgtgttg | Purandare et al |
| | | | | | (SEQ ID NO 54) | |
| NFex3.2 | (95°1′-60°1′-72°1′) × 35 | 5 × Dy | 237 | bp | tggtccacatctgtactttg | |
| | | | | | (SEQ ID NO 55) | |
| NFex3.1cy | 72° 10′ | 1UtaqBRL | | | | |
| | 4° | TV = 25 μl | | | | |
| NFex4Ax.1 | 95° 5′ | 100 ng | 517 | bp | ttaaatctaggtggtgtgt | own |
| | | | | | (SEQ ID NO 56) | |
| NFex4Ax.2 | (95°45″-54°30″- | 10 × Boeh | | | aaactcatttctctggag | own |
| | 72°30″) × 35 | | | | (SEQ ID NO 57) | |
| | 72°10′ | 1UPltaq | | | | |
| | 4° | 25 μl | | | | |
| NFex4B.1wal | 95°5′ | 100 ng | | | ggcttcctgaagtgctgggat | Walace M., pers. |
| | | | | | (SEQ ID NO 58) | comm. |
| NFex4B.2wal | 95°45″-63°25″- | 10 × CS | 305 | bp | ccagtttggtgttctagttcagca | Walace M., pers. |
| | 72°25″) × 35 | | | | (SEQ ID NO 59) | comm. |
| | 72° 10′ | 1UPlTaq | | | | |
| NFex4B.1Vfl | 4° | TV = 25μl | | | fluo-gtgagataccacacctgtccc | Viskochil |
| (seq 60°) | | | | | (SEQ ID NO 60) | |
| NFex4c.1 | HS | 100 ng | | | tttcctagcagacaactatcga | Purandare et al |
| | | | | | (SEQ ID NO 61) | |
| NFex4c.2 | (95°1′-60°1′-72°1′) × 45 | 5 × Dy | 283 | bp | catcaaaaaaaaaattttaataccag | |
| | | | | | (SEQ ID NO 62) | |
| NFex4c.1fl | 72° 10′ | 1UtaqBRL | | | | |
| | 4° | TV = 25 μl | | | | |
| NFex5.1fl | HS | 200 ng | | | fluo-catgtggttctttatttataggc | Hoffmeyer et al |
| | | | | | (SEQ ID NO 63) | |
| NFex5.2 | (95°1′-52′1′-72°1′) × 35 | 10 × CS | 113 | bp | tcaatcgtatccttaccagcc | |
| | | | | | (SEQ ID NO 64) | |
| | 72° 10′ | 1UtaqBRL | | | | (primers lay |
| | | | | | | within |
| | | | | | | exon) |
| | 4° | TV = 25 μl | | | | |
| NFex6.1 | HS | 100 ng | | | catgtttatcttttaaaaatgttgcc | Purandare et al |
| | | | | | (SEQ ID NO 65) | |
| NFex6.2 | (95°1′-64°1′-72°1′) × 35 | 10 × Boeh | 299 | bp | ataatggaaataattttgccctcc | |
| | | | | | (SEQ ID NO 66) | |
| | 72° 10′ | 1UtaqBRL | | | | |
| | 4° | TV = 25 μl | | | | |
| NFex7.1 | HS | 100 ng | | | tgctataatattagctacatctgg | Purandare et al |
| | | | | | (SEQ ID NO 67) | |
| NFex7.2 | (95°1′-58°1′-72°1′) × 35 | 5 × Dy | 373 | bp | Cctatgaacttatcaacgaagag | |
| | | | | | (SEQ ID NO 68) | |
| NFex7.1cy | 72° 10′ | 1UtaqBRL | | | | |
| | 4° | TV = 25 μl | | | | |
| NFex7 + 8.1 | HS | 100 ng | | | tgctataatattagctacatctgg | Purandare et al |
| | | | | | (SEQ ID NO 69) | |
| NFex7 + 8.2 | (95°1′-55°1′-72°1′) × 35 | 10 × Boeh | 880 | bp | ctagtctttctgtttataaaggat | |
| | | | | | (SEQ ID NO 70) | |
| | 72° 10′ | 1UtaqBRL | | | | |
| | 4° | TV = 25 μl | | | | |
| NFex9.1br | HS | 100 ng | | | tccgctgtggctcagaacac | Purandare et al |
| | | | | | (SEQ ID NO 71) | |
| NFex9.2br | (95°1′-63°1′-72°1′) × 40 | 10 × Boeh | 335 | bp | agtagaagaggatgcacagcc | |
| | | | | | (SEQ ID NO 72) | |
| | 72° 10′ | 1UtaqBRL | | | | |
| | 4° | TV = 25 μl | | | | |
| NFex9.1 | 95°5′ | 100 ng | | | tttgacctcatttgtattactgag | |
| | | | | | (SEQ ID NO 73) | |
| NFex9.2 | (95°1′-60°1′-72°1′) × 35 | 10 × CS | 249 | bp | agaaccttttgaaaccaagagtg | Purandare et al |
| | | | | | (SEQ ID NO 74) | |
| | 72°10′ | 1UPltaq | | | | |
| | 4° | TV = 25 μl | | | | |
| NFex10A.1 | HS | 100 ng | | | acgtaattttgtactttttcttcc | Purandare et al |
| | | | | | (SEQ ID NO 75) | 1995 |
| NFex10A.2 | (95°1′-58°1′-72°1′) × 35 | 10 × Boeh | 222 | bp | caatagaaaggaggtgagattc | |
| | | | | | (SEQ ID NO 76) | |
| NEex10A.1fl | 72° 10′ | 1UtaqBRL | | | | |
| NFex10A.2cy | 4° | TV = 25 μl | | | | |
| NFex10C.1 | HS | 100 ng | | | cttggtaccctttagcagtcac | Purandare et al |
| | | | | | (SEQ ID NO 77) | |
| NFex10C.2 | (95°1′-59°1′-72°1′) × 35 | 5 × Dy | 379 | bp | ccttctttctccatggag | |
| | | | | | (SEQ ID NO 78) | |
| NFex10C.1cy | 72° 10′ | 1UtaqBRL | | | | |
| | 4° | TV = 25 μl | | | | |
| NEex11.1 | HS | 100 ng | | | gtactccagtgttatgtttacc | Purandare et al |
| | | | | | (SEQ ID NO 79) | 1995 |
| NFex11.2 | (95°1′-55°1′-72°1′) × 40 | 5 × Dy | 190 | bp | taaagttgaaatttaaaaattaaagtac | |
| | | | | | (SEQ ID NO 80) | |
| NFex11.1cy | 72° 10′ | 1UtaqBRL | | | | |
| NFex11.2cy | 4° | TV = 25 μl | | | | |
| NFex12A.1 | HS | 100 ng | | | acttgtattcattatgggagaatg | MRC (Maynard) |
| | | | | | (SEQ ID NO 81) | |
| NFex12A.2 | (95°1′-60°1′-72°1′) × 35 | 5 × Dy | 284 | bp | agtaatctctcaccattaccattc | |
| | | | | | (SEQ ID NO 82) | |
| NFex12A.2cy | 72° 10′ | 1UtaqBRL | | | | |
| | 4° | TV = 25 μl | | | | |
| NFex12B.1 | HS | 100 ng | | | tttctagtgaatctccttcaagt | Purandare et al |
| | | | | | (SEQ ID NO 83) | |
| NFex12B.2 | (95°1′-59°1′-72°1′) × 40 | 5 × Dy | 382 | bp | atgaaatttaccaaatttcattcag | |
| | | | | | (SEQ ID NO 84) | |
| NFex12B.1cy | 72° 10′ | 1UtaqBRL | | | | |
| | 4° | TV = 25 μl | | | | |
| NFex13X.1cy | 95°5′ | 100 ng | | | cy-gagttattgtatgcggagac | own |
| | | | | | (SEQ ID NO 85) | |
| NFex13X.2 | (95°1′-55°1′-72°1′) × 30 | 10 × CS,B,D | 494 | bp | ttgaatttcccctgtaaac | own |
| | | | | | (SEQ ID NO 86) | |
| NFex13.1fl | 72°10′ | 1UplTaq | | | fluo-cacagtttattgcattgttagat | Purandare et al |
| (seq bij°) | | | | | (SEQ ID NO 87) | |
| | 4° | TV = 25 μl | | | | |
| NFex14.1 | HS | 100 ng | | | tccttttgggtggagcttatc | Purandare et al |
| | | | | | (SEQ ID NO 88) | |
| NFex14.2 | (95°1′-59°1′-72°1′) × 35 | 10 × Boeh | 286 | bp | tatacttgtaatatgcacgtatc | |
| | | | | | (SEQ ID NO 89) | |
| NFex14.1cy | 72° 10′ | 1UtaqBRL | | | | |
| | 4° | TV = 25 μl | | | | |
| NFex15.1fl | HS | 100 ng | | | fluo-tgtgatcaggaatagcttttgaa | Purandare et al |
| | | | | | (SEQ ID NO 90) | |
| NFex15.2 | (95°1′-57°1′-72°1′) × 40 | 5 × Dy | 276 | bp | ttaacagataaaagtcaactttac | |
| | | | | | (SEQ ID NO 91) | |
| | 72° 10′ | 1UtaqBRL | | | | |
| | 4° | TV = 25 μl | | | | |
| NFex16.1 | HS | 100 ng | | | tggataaagcataatttgtcaagt | Purandare et al |
| | | | | | (SEQ ID NO 92) | |
| NFex16.2 | (95°1′-58°1′-72°1′) × 35 | 5 × Dy | 549 | bp | tagagaaaggtgaaaaataagag | |
| | | | | | (SEQ ID NO 93) | |
| NFex16.1cy | 72° 10′ | 1UtaqBRL | | | | |
| NFex16seq | 4° | TV = 25 μl | | | cy-ccagtcagtgacgtaaggg | own |
| cy | | | | | (SEQ ID NO 94) | |
| NFex17.1 | HS | 100 ng | | | ctctgtgtgtttagatcagtca | Purandare et al |
| | | | | | (SEQ ID NO 95) | |
| NFex17.2 | (95°1′-55°1′-72°1′) × 35 | 10 × Boeh | 319 | bp | tttatcaattactaccagtatcag | |
| | | | | | (SEQ ID NO 96) | |
| NFex17.1cy | 72° 10′ | 1UtaqBRL | | | | |
| | 4° | TV = 25 μl | | | | |
| NFex18.1 | HS | 200 ng | | | tagtaaggtagccagaagttgtgt | MRC (Maynard) |
| | | | | | (SEQ ID NO 97) | |
| NFex18.2 | (95°1′-60°1′-72°1′) × 35 | 10 × Boeh | 320 | bp | atttacaaaaccctacattgctc | |
| | | | | | (SEQ ID NO 98) | |
| NFex18.1cy | 72° 10′ | 1UtaqBRL | | | | |
| | 4° | TV = 25 μl | | | | |
| NFex19A.1 | HS | 100 ng | | | tcatgtcacttaggttatctgg | Purandare et al |
| | | | | | (SEQ ID NO 99) | |
| NFex19A.2 | (95°1′-53°1′-72°1′) × 40 | 5 × Dy | 272 | bp | tgtaattaagtagttataactctc | |
| | | | | | (SEQ ID NO 100) | |
| NFex19A.1cy | 72° 10′ | 1UtaqBRL | | | cy-tcatgtcacttaggttatctgg | |
| | | | | | (SEQ ID NO 101) | |
| | 4° | TV = 25 μl | | | | |
| NFex19B.1x | 95°5′ | 100 ng | | | attaccttctccccatttga | own |
| | | | | | (SEQ ID NO 102) | |
| NFex19B.2x | (95°1′-55°1′-72°1′) × 30 | 10 × boeh | 371 | bp | ggctttatttgctttttgc | |
| | | | | | (SEQ ID NO 103) | |
| NFex19B.1x | 72°10′ | 1Upltaq | | | | |
| cy | | | | | | |
| NFex19B.2x | 4° | TV = 25 μl | | | | |
| cy | | | | | | |
| NFex20.1 | HS | 100 ng | | | ccaccctggctgattatcg | Purandare et al |
| | | | | | (SEQ ID NO 104) | |
| NFex20.2 | (95°1′-62°1′-72°1′) × 35 | 10 × Dy | 402 | bp | taatttttgcttctcttacatgc | |
| | | | | | (SEQ ID NO 105) | |
| NFex20.1cy | 72° 10′ | 1UtaqBRL | | | | |
| | 4° | TV = 25 μl | | | | |
| NFex20- | 95°5′ | 100 ng | | | ctatatcaggtaaaatcatgtccaac | Fahsold et al |
| 21.1 | | | | | (SEQ ID NO 106) | |
| NFex20- | (95°1′-60°1′-72°1′)*35 | 10 × boeh | | | gatttgctatgtgccagggac | |
| 21.2 | | | | | (SEQ ID NO 107) | |
| | 72°10′ | 1Upltaq | | | | |
| | 4° | TV = 25 μl | | | | |
| NFex21xx.1 | 95° 5′ | 100 ng | | | gtcaaacttactcaatgcc | own |
| | | | | | (SEQ ID NO 108) | |
| NFex21xx.2 | (95°45″-54°30″- | 10 × Boeh | 542 | bp | caaccacttccctacag | own |
| | 72°30″) × 31 | | | | (SEQ ID NO 109) | |
| NFex21xx.1 | 72°10′ | 1UPltaq | | | | |
| cy | | | | | | |
| NFex21x1.cy | 4° | 25 μl | | | cy-aactggcatgtaagagaag | own |
| | | | | | (SEQ ID NO 110) | |
| NFex22.1 | HS | 100 ng | | | tgctactctttagcttcctac | Purandare et al |
| | | | | | (SEQ ID NO 111) | |
| NFex22.2 | (95°1′-58°1′-72°1′) × 35 | 10 × CS | 331 | bp | ccttaaaagaagacaatcagcc | |
| | | | | | (SEQ ID NO 112) | |
| NFex22.1cy | 72° 10′ | 1UtaqBRL | | | | |
| | 4° | TV = 25 μl | | | | |
| NFex23AN.1 | 95°5′ | 100 ng | 382 | bp | gattgggtctcaacatttc | |
| | | | | | (SEQ ID NO 113) | |
| NFex23AN.2 | (95°1′-57°1′-72°1′) × 35 | 10 × Dy | | | cy-aataggctgaagtgaagatantc | own? |
| cy | | | | | (SEQ ID NO 114) | |
| | 72°10′ | 1UplTaq | | | | |
| | 4° | TV = 25 μl | | | | |
| NFex23.1.1 | HS | 100 ng | | | tttgtatcattcattttgtgtgta | Purandare et al |
| | | | | | (SEQ ID NO 115) | |
| NFex23.1.2 | (95°1′-60°1′-72°1′) × 35 | 5 × Dy | 282 | bp | aaaaacacggttctatgtgaaaag | |
| | | | | | (SEQ ID NO 116) | |
| NFex23.1. | 72° 10′ | 1UtaqBRL | | | | |
| 1cy | 4° | TV = 25 μl | | | | |
| NFex23.2.1 | HS | 100 ng | | | cttaatgtctgtataagagtctc | Purandare et al |
| | | | | | (SEQ ID NO 117) | |
| NFex23.2.2 | (95°1′-52°1′-72°1′) × 35 | 10 × CS | 268 | bp | actttagattaataatggtaatctc | |
| | | | | | (SEQ ID NO 118) | |
| NFex23.2.1 | 72° 10′ | 1UtaqBRL | | | | |
| cy | 4° | TV = 25 μl | | | | |
| NFex24b.1 | HS | 100 ng | | | ttgaactctttgttttcatgtctt | Purandare et al |
| | | | | | (SEQ ID NO 119) | |
| NFex24b.2 | (95°1′-57°1′-72°1′) × 35 | 5 × Dy | 267 | bp | ggaatttaagatagctagattatc | |
| | | | | | (SEQ ID NO 120) | |
| NFex24MRC, | 72° 10′ | 1UtaqBRL | | | | |
| not ok | 4° | TV = 25 μl | | | | |
| NFex25b.1 | HS | 100 ng | | | aatataataattatatttgggaaggt | Purandare et al |
| | | | | | (SEQ ID NO 121) | |
| NFex25b.2 | (95°1′-57°1′-72°1′) × 35 | 10 × Boeh | 338 | bp | gaaaatatttgattcaaacagagc | |
| | | | | | (SEQ ID NO 122) | |
| NFex25b.1cy | 72° 10′ | 1UtaqBRL | | | cy-aatataataattatatttgggaaggt | |
| | | | | | (SEQ ID NO 123) | |
| | 4° | TV = 25 μl | | | | |
| NFex25L.1 | HS | 100 ng | | | cattttattatagcagatgtc | own |
| | | | | | (SEQ ID NO 124) | |
| NFex25L.2 | (95°45″-57°45″- | 5 × Dy | 534 | bp | acttacacaggaacttcat | |
| | 72°45″) × 35 | | | | (SEQ ID NO 125) | |
| | 72° 10′ | 1UtaqBRL | | | | |
| | 4° | TV = 25 μl | | | | |
| NFex26.1fl | HS | 100 ng | | | fluo-gctttgtctaatgtcaagtcac | Purandare et al |
| | | | | | (SEQ ID NO 126) | |
| NFex26.2 | (95°1′-58°1′-72°1′) × 35 | 5 × Dy | 342 | bp | ttaaacggagagtgttcactatc | |
| | | | | | (SEQ ID NO 127) | |
| | 72° 10′ | 1UtaqBRL | | | | |
| | 4° | TV = 25 μl | | | | |
| NFex27A.1 | HS | 100 ng | | | gttacaagttaaagaaatgtgtag | Purandare et al |
| | | | | | (SEQ ID NO 128) | |
| NFex27A.2 | (95°30″-62°30″- | 10 × Boeh | 300 | bp | ctaacaagtggcctggtggcaaac | |
| | 72°30″) × 45 | | | | (SEQ ID NO 129) | |
| NFex27A.1fl | 72° 5′ | 1UtaqBRL | | | | |
| | 4° | TV = 25 μl | | | | |
| NFex27B.1fl | does not work | | | | fluo-tttattgtttatccaattatagactt | Purandare et al |
| | | | | | (SEQ ID NO 130) | |
| NFex27B.2 | | | | | tcctgttaagtcaactgggaaaaac | |
| | | | | | (SEQ ID NO 131) | |
| NFex28 Aber | 95°5′ | 200 ng | | | cactgctaataatctttgtcttttttgtc | Abernathy |
| .1 | | | | | (SEQ ID NO 132) | |
| NFex28 Aber | (95°1′-65°1′-72°1′) × 40 | 10 × Boeh | 501 | bp | cgtttacaaaacacagactggaactta | |
| .2 | | | | | (SEQ ID NO 133) | |
| NFex28 Aber | 72°10′ | 1UPltaq | | | | |
| .1cy | | | | | | |
| NFex28 Aber | 4° | TV = 25 μl | | | | |
| .2cy | | | | | | |
| NFex28.1fl | | | | | ttccttaggttcaaaactggtca | own |
| (nested) | | | | | (SEQ ID NO 134) | |
| NFex29L1 | H.S. | 100 ng | | | tacaatggtgggaactc | own |
| | | | | | (SEQ ID NO 135) | |
| NFex29.L2 | (95°1′-56°45″- | 10 × CS | 567 | bp | atattaaggtagaggctgttt | own |
| | 72°45″) × 35 | | | | (SEQ ID NO 136) | |
| NFex29L1cy | 72°10′ | 1UtaqBRL | | | | |
| NFex29L2cy | 4° | TV = 25 μl | | | | |
| NFex29.1cy | | | | | cy-attcttctccacttcaccc | MRC (Shen) |
| (nested) | | | | | (SEQ ID NO 137) | |
| NFex29.2cy | | | | | cy-cccaaatcaaactgaagaga | MRC |
| (nested) | | | | | (SEQ ID NO 138) | |
| NFex30x.1 | 95°5′ | 100 ng | 350 | bp | tggaactataaggaaaaa | own |
| | | | | | (SEQ ID NO 139) | |
| NFex30x.2cy | (95°1′-50°1′- | 10 × CS | | | cy-aaagtcttcactggaaa | own |
| | 72°1′30″) × 30 | | | | (SEQ ID NO 140) | |
| | 72°10′ | 1UPITaq | | | | |
| NFex30.2fl | 4° | TV = 25 μl | | | | |
| (3)(nested) | | | | | | |
| NFex31.1(4) | HS | 100 ng | | | ataattgttgatgtgattttcattg | Cawthon |
| | | | | | (SEQ ID NO 141) | |
| NFex31.2f1 | (93°1′-63°1′- | 10 × Koh | 424 | bp | fluo-aattttgaaccagatgaagag | |
| (4) | 72°1′30″) × 31 | | | | (SEQ ID NO 142) | |
| NFex31.1cy | 72° 5′ | 1UtaqBRL | | | | |
| | 4° | TV = 25 μl | | | | |
| NFex32.1 | HS | 100 ng | | | ggtagagtgattaaaacatg | |
| | | | | | (SEQ ID NO 143) | |
| NFex32.2 | (95°1′-51°1′-72°1′) × 35 | 10 × CS | 220 | bp | tatgctatagtacagaaggc | Rina Wu |
| | | | | | (SEQ ID NO 144) | |
| NFex32.1cy | 72° 10′ | 1UtaqBRL | | | | |
| | 4° | TV = 25 μl | | | | |
| NFex33.1fl | HS | 100 ng | | | fluo-catatctgttttatcatcaggagg | |
| | | | | | (SEQ ID NO 145) | |
| NFex33.2 | (95°1′-61°1′-72°1′) × 35 | 5 × Dy | 462 | bp | aagtaaaatggagaaaggaactgg | Cawthon |
| | | | | | (SEQ ID NO 146) | |
| NFex33.2cy | 72° 10′ | 1UtaqBRL | | | | |
| | 4° | TV = 25 μl | | | | |
| NFex34.1fl | HS | 100 ng | | | fluo-caaaatgaaacatggaactttaga | |
| | | | | | (SEQ ID NO 147) | |
| NFex34.2 | (95°1′-57°1′-72°1′) × 35 | 5 × Dy | 400 | bp | taagcattaagtacaaatagcaca | Cawthon |
| | | | | | (SEQ ID NO 148) | |
| | 72° 10′ | 1UtaqBRL | | | | |
| | 4° | TV = 25 μl | | | | |
| NFex35.1fl | HS | 100 ng | | | fluo-agaagccaaaatgataagaa | |
| | | | | | (SEQ ID NO 149) | |
| NFex35.2 | (95°1′-52°1′-72°1′) × 35 | 10 × CS | 495 | bp | acccaaagacaacaagag | |
| | | | | | (SEQ ID NO 150) | |
| | 72° 10′ | 1UtaqBRL | | | | |
| | 4° | TV=25 μl | | | | |
| NFex36.1 | HS | 100 ng | | | ggaccagtggacagaac | own |
| | | | | | (SEQ ID NO 151) | |
| NFex36.2fl | (95°1′-55°1′-72°1′) × 35 | 10 × Boeh | 345 | bp | fluo-atatgctttacaacttgagaa | |
| | | | | | (SEQ ID NO 152) | |
| | 72° 10′ | 1UtaqBRL | | | | |
| | 4° | TV = 25 μl | | | | |
| NFex37.1 | HS | 100 ng | | | tacattaagctagctaccaa | own |
| | | | | | (SEQ ID NO 153) | |
| NFex37.2fl | (95°1′-54°1′-72°1′) × 35 | 10 × Boeh | 460 | bp | fluo-cgcttgagaacatactatcc | |
| | | | | | (SEQ ID NO 154) | |
| | 72° 10′ | 1UtaqBRL | | | | |
| | 4° | TV = 25 μl | | | | |
| NFex38.1fl | HS | 100 ng | | | fluo-ccagctaacagtgtctt | own |
| | | | | | (SEQ ID NO 155) | |
| NFex38.2 | (95°1′-50°1′-72°1′) × 35 | 10 × Boeh | 474 | bp | aaggaaatatactcacaataa | |
| | | | | | (SEQ ID NO 156) | |
| | 72° 10′ | 1UtaqBRL | | | | |
| | 4° | TV = 25 μl | | | | |
| NFex39.1fl | HS | 100 ng | | | fluo-gaacctaatcaaccatctc | |
| | | | | | (SEQ ID NO 157) | |
| NFex39.2 | (95°1′-52°1′-72°1′) × 35 | 5 × Dy | 286 | bp | ttgcatttaaagtaagacat | |
| | | | | | (SEQ ID NO 158) | |
| | 72° 10′ | 1UtaqBRL | | | | |
| | 4° | TV = 25 μl | | | | |
| NFex40.1fl | HS | 100 ng | | | fluo-cctttccttgcagagttgtta | |
| | | | | | (SEQ ID NO 159) | |
| NFex40.2 | (95°1′-57°1′-72°1′) × 35 | 5 × Dy | 371 | bp | caccactaaaggactagactgt | |
| | | | | | (SEQ ID NO 160) | |
| | 72° 10′ | 1UtaqBRL | | | | |
| | 4° | TV = 25 μl | | | | |
| NFex41.1fl | HS | 100 ng | | | fluo-ttcatcctgttttaagtcacacttg | |
| | | | | | (SEQ ID NO 161) | |
| NFex41.2 | (95°1′-61°1′-72°1′) × 40 | 10 × Boeh | 273 | bp | ttgcctccattagttggaaaattg | Abernathy et al |
| | | | | | (SEQ ID NO 162) | |
| NFex41.1 | 72° 10′ | 1UtaqBRL | | | ttcatcctgttttaagtcacacttg | |
| | | | | | (SEQ ID NO 163) | |
| | 4° | TV = 25 μl | | | | |
| NFex42.1fl | HS | 100 ng | | | fluo-cttggaaggagcaaacgatggttg | |
| | | | | | (SEQ ID NO 164) | |
| NFex42.2 | (95°1′-61°1′-72°1′) × 35 | 10 × Boeh | 356 | bp | caaaaactttgctacactgacatgg | Abernathy et al |
| | | | | | (SEQ ID NO 165) | |
| | 72°10′ | 1UTaq BRL | | | | |
| | 4° | TV = 25 μl | | | | |
| NFex43.1fl | HS | 100 ng | | | fluo-agacactgtagttaatgaacttgc | |
| | | | | | (SEQ ID NO 166) | |
| NFex43.2 | (95°1′-60°1′-72°1′) × 35 | 10 × Boeh | 224 | bp | catgtactctcccaccttattttc | Abernathy et al |
| | | | | | (SEQ ID NO 167) | |
| NFex43.1Cy | 72° 5′ | Taq ST | | | cy-agacactgtagttaatgaacttgc | |
| | | 1/20 | | | (SEQ ID NO 168) | |
| | 4° | TV = 25 μl | | | | |
| NFex44.1fl | HS | 100 ng | | | fluo-cacgttaattccctatcttgctgc | F Shen et al |
| | | | | | (SEQ ID NO 169) | |
| NFex44.2 | (95°1′-65°1′-72°1′) × 35 | 10 × Boeh | 200 | bp | taaaaatttgagggtgggggactc | R? |
| | | | | | (SEQ ID NO 170) | |
| | 72° 5′ | Taq ST | | | | |
| | | 1/20 | | | | |
| | 4° | TV = 25 μl | | | | |
| NFex45.1fl | HS | 100 ng | | | fluo-catgaataggatacagtcttctac | |
| | | | | | (SEQ ID NO 171) | |
| NFex45.2 | (95°1′-60°1′-72°1′) × 35 | 10 × Boeh | 269 | bp | cacattactgggtaagcatttaac | Abernathy |
| | | | | | (SEQ ID NO 172) | |
| NFex45.2fl | 72° 5′ | 1UtaqBRL | | | | |
| NFex45.1bio | 4° | TV = 25 μl | | | | |
| NFex46.1fl | HS | 100 ng | | | fluo-gggaatgtatattatgttttccac | |
| | | | | | (SEQ ID NO 173) | |
| NFex46.2 | (95°1′-60°1′-72°1′) × 35 | 5 × Dy | 275 | bp | atgttaggaagttcatcaaccatc | Abernathy |
| | | | | | (SEQ ID NO 174) | |
| NFex46.2cy | 72° 5′ | 1UtaqBRL | | | | |
| | 4° | TV = 25 μl | | | | |
| NFex47.1 | HS | 100 ng | | | ctgttacaattaaaagataccttgc | MRC (Upadhyaya) |
| | | | | | (SEQ ID NO 175) | |
| NFex47.2 | (95°1′-60°1′-72°2′) × 35 | 5 × Dy | 185 | bp | tgtgtgttcttaaagcaggcatac | MRC (Upadhyaya) |
| | | | | | (SEQ ID NO 176) | |
| | 72° 10′ | 1UtaqBRL | | | | |
| | 4° | TV = 25 μl | | | | |
| NFex48.1 | HS | 100 ng | | | ttttggcttcagatggggatttac | MRC (Upadhyaya) |
| | | | | | (SEQ ID NO 177) | |
| NFex48.2 | (95°1′-65°1′-72°1′) × 35 | 5 × Dy | 352 | bp | aagggaattcctaatgttggtgtc | MRC (Upadhyaya) |
| | | | | | (SEQ ID NO 178) | |
| | 72° 10′ | 1UtacBRL | | | | |
| | 4° | TV = 25 μl | | | | |
| NFex48A.1fl | HS | 100 ng | | | fluo-atctagtatctaattgtatttcacc | Li et al |
| | | | | | (SEQ ID NO 179) | |
| NFex48A.2 | (95°1′-60°1′-72°2′) × 40 | 5 × Dy | | | gcagactgagcttacagggac | |
| | | | | | (SEQ ID NO 180) | |
| | 72° 10′ | 1UtaqBRL | | | | |
| | 4° | TV = 25 μl | | | | |

Legend

| | | | | |
|---|---|---|---|---|
| Labels | fl | Fluoresceïn label | | |
| | cy | Cy5 label | | |
| | bio | Biothine label | | |
| Buffers | Dy | Dynazyme buffer | 5X buffer contains | 50 mM Tris- |
| | | | | HCl(pH8.8) |
| | | | | 250 mM KCl |
| | | | | 0.5% Triton X- |
| | | | | 100 |
| | | | | 7.5 mM MgCl₂ |
| | Boeh | Boehringer buffer | 10X buffer contains | 100 mM Tris HCl |
| | | | | 15 mM MgCl₂ |
| | | | | 500 mM KCl |
| | | | | pH 8.3 (20° C.) |
| | Koh | Kohan buffer | 10X buffer contains | 166 mM |
| | | | | (NH₄)₂SO₄ |
| | | | | 670 mM Tris- |
| | | | | HCl(pH8.8) |
| | | | | 67 mM MgCl₂ |
| | | | | 100 mM β- |
| | | | | mercaptoethanol |
| | | | | 67 μM EDTA |
| | | | | 1700 μg BSA/ml |
| | | | | 5% DMSO |
| | CS | AC-Syvänen buffer | 10X buffer contains | 200 mM Tris- |
| | | | | HCl(pH8.8) |
| | | | | 15 mM MgCl₂ |
| | | | | 150mM |
| | | | | (NH₄)₂SO₄ |
| | | | | 1% Triton X-100 |
| | | | | 0.1% gelatin |
| others | HS | Hot Start | 95°C. 5 min, 80°C. 5 min while adding the | |
| | | | polymerase | |

| | |
|---|---|
| 1 UtaqBRL | 1 unit of Taq Polymerase from BRL |
| 1UPltaq | 1 unit of Platinum Taq from BRL |

| | | |
|---|---|---|
| references | Shen et al | Neurofibromatosis type 1 (NF1): the search for mutations |
| | | by PCR-heteroduplex analysis on Hydrolink gels. Hum |
| | | Mol Gen, 1993, Vol2, No 11, 1861-1864 |
| | Purandare et al | Identification of Neurofibromatosis 1 (NF1) Homologous |
| | | Loci by Direct Sequencing, Fluorescence in Situ |
| | | Hybridization, and PCR Amplification of Somatic Cell |
| | | Hybrids. Genomics; 1995: 30, 476-485 |
| | Wallace et al | personal communication |
| | Sawada et al | Identification of NF1 mutation in both alleles of a dermal |
| | | neurofibroma. Nat Genet;1996 Sep:14(1):110-2 |
| | Hoffmeyer et al | An Rsal polymorphism in the transcribed region of the |
| | | neurofibromatosis (NF1)-gene. Hum Genet; 1994:93:481- |
| | | 482 |
| | Fahsold et al | Minor Lesion Mutational Spectrum of the NF1 Gene Does |
| | | Not Explain Its High Mutability but Points to a Functional |
| | | Domain Upstream of the Gap-Related domain. Am J |
| | | Hum Genet; 2000:66:790-818 |
| | Abernathy et al | NF1 mutation analysis using a combined |
| | | heteroduplex/SSCP approach. Hum Mut 1997;9:548-554 |
| | Cawthon et al | A Major Segment of the Neurofibromatosis Type 1 Gene: |
| | | cDNA, Genomic Stucture, and Point Mutations. Cell; |
| | | 1990:62:193-201 |
| | MRC | Medical Research Counsil, UK, HGMP primers database |
| | Rina Wu et al | PhD dissertation KUL |
| | Li et al | Genomic Organization of the Neurofibromatosis 1 Gene |
| | | (NF1). Genomics 1995;25:9-18 |

TABLE 4

HA-PCR primers and conditions

| | | | |
|---|---|---|---|
| | primer sequences | Fragment | |
| Exon | (5′ to 3′) | size | Tₐ(° C.)¹ |

| | | | |
|---|---|---|---|
| 2 | TTTCAATGGCAAGTAAGT | 340 | 54 |
| | GTTATATCCAAAGTCCACA | | |
| 4a | TTAAATCTAGGTGGTGTGT | 517 | 54 |
| | AAACTCATTTCTCTGGAG | | |
| 13 | GAGTTATTGTATGCGGAGAC | 494 | 55 |
| | TTGAATTTCCCCTGTAAAC | | |
| 19b | ATTACCTTCTCCCCATTTGA | 371 | 55 |
| | GGCTTTATTTGCTTTTTGC | | |
| 21 | GTCAAACTTACTCAATGCC | 542 | 54 |
| | CAACCACTTCCCTACAG | | |
| 23a | GATTGGGTCTCAACATTTC | 382 | 57 |
| | AATAGGCTGAAGTGAAGATANTC | | |
| 29 | TACAATGGTGGGAACTC | 567 | 56 |
| | ATATTAAGGTAGAGGCTGTTT | | |
| 30 | TGGAACTATAAGGAAAAA | 350 | 50 |
| | AAAGTCTTCACTGGAAA | | |
| 32 | GGTAGAGTGATTAAAACATG | 220 | 51 |
| | TATGCTATAGTACAGAAGGC | | |
| 35 | AGAAGCCAAAATGATAAGAA | 495 | 52 |
| | ACCCAAAGACAACAAGAG | | |
| 36 | GGACCAGTGGACAGAAC | 345 | 55 |
| | ATATGCTTTACAACTTGAGAA | | |
| 37 | TACATTAAGCTAGCTACCAA | 460 | 54 |
| | CGCTTGAGAACATACTATCC | | |
| 38 | CCAGCTAACAGTGTCTT | 474 | 50 |
| | AAGGAAATATACTCACAATAA | | |
| 39 | GAACCTAATCAACCATCTC | 286 | 52 |
| | TTGCATTTAAAGTAAGACAT | | |
| 40 | CCTTTCCTTGCAGAGTTGTTA | 371 | 57 |
| | CACCACTAAAGGACTAGACTGT | | |
| 44 | CACGTTAATTCCCTATCTTGCTGC | 200 | 65 |
| | TAAAAATTTGAGGGTGGGGGACTC | | |

¹Tₐ(° C.), annealing temperature for the primers during PCR

TABLE 5
Mutations detected by analysis of the whole coding region of the NF1 gene by PTT and HA;
analysis of 67 unrelated NF1 patients.

| | | | | Codon | |
|---|---|---|---|---|---|
| Patient | Genomic Mutation¹ | Effect on cDNA | Codon Change | Number | Exon/Intron |
| NF-004 | 247C > T | 247C > T | Q > X | 83 | 3 |
| NF-023 | 278G > A | 278G > A | C > Y | 93 | 3 |
| NF-036 | 560G > A | 560G > A | C > Y | 187 | 4b |
| NF-056 | 574C > T | 574C > T | R > X | 192 | 4b |
| NF-029 | 603-604insT | 603-604insT | frameshift | 202 | 4c |
| NF-027 | 910C > T | 910C > T; | R > X | 304 | 7 |
| | | 888del174 | | | |
| NF-064 | 910C > T | 910C > T; | R > X | 304 | 7 |
| | | 888del174 | | | |
| NF-024 | 987-988insA | 987-988insA | frameshift | 330 | 7 |
| NF-048 | 1275G > A | 1275G > A | W > X | 425 | 10a |
| NF-042 | 1318C > T | 1318C > T | R > X | 440 | 10a |
| NF-057 | 1318C > T | 1318C > T | R > X | 440 | 10a |
| NF-030 | 1381C > T | 1381C > T | R > X | 461 | 10a |
| NF-026 | 1381C > T | 1381C > T | R > X | 461 | 10a |
| NF-017 | 1466A > G | 1465del62 | Y > C | 489 | 10b |
| NF-012 | 1466A > G | 1465del62 | Y > C | 489 | 10b |
| NF-016 | 1465-1466insC | 1466insC | Y > X | 489 | 10b |
| NF-033 | 1570G > T | 1570G > T | E > X | 524 | 10c |
| NF-052 | 1607C > A | 1607C > A | S > X | 536 | 10c |
| NF-045 | IVS12a + 1G > T | 1641del204 | none | NA | IVS12a |
| NF-034 | 2033-2034insC | 2033insC | frameshift | 678 | 13 |
| NF-035 | 2033-2034insC | 2033insC | frameshift | 678 | 13 |
| NF-010 | 2540T > C | 2540T > C | L > P | 847 | 16 |
| NF-051 | IVS16 + 2del6 | 2617del233 | frameshift | NA | IVS16 |
| NF-028 | IVS16 − 6del4 | 2850del140 | frameshift | NA | IVS16 |
| NF-014 | 2875C > T | 2875C > T | Q > X | 959 | 17 |
| NF-011 | 2887C > T | 2887C > T | Q > X | 963 | 17 |
| NF-001 | 2970-2972delAAT | 2970-2972delAAT | delM | 991 | 17 |
| NF-053 | 3193delC | 3193delC | frameshift | 1065 | 19a |
| NF-063 | 3277G > A | 3277G > A, 3274del40 | V > M; | 1093 | 19b |
| | | | frameshift | | |
| NF-025 | IVS19b − 3C > G | 3314del182 | frameshift | NA | IVS19b |
| NF-039 | 3367G > T | 3367G > T | E > X | 1123 | 20 |
| NF-046 | 3520C > T | 3520C > T | Q > X | 1174 | 21 |
| NF-059 | 3826C > T | 3826C > T | R > X | 1276 | 22 |
| NF-041 | 4084C > T | 4084C > T | R > X | 1362 | 23.2 |
| NF-054 | 4084C > T | 4084C > T | R > X | 1362 | 23.2 |
| NF-044 | IVS26 − 2A > T | 4515-14 ins 14; | frameshift | NA | IVS26 |
| | | 4515-17ins17 | | | |
| NF-021 | 4537C > T | 4537C > T | R > X | 1513 | 27a |
| NF-006 | 4537C > T | 4537C > T | R > X | 1513 | 27a |
| NF-058 | IVS27b − 2A > T | 4772del433; 4772del293 | frameshift | NA | IVS27b |
| NF-049 | 5033delG | 5033delG | frameshift | 1678 | 28 |
| NF-009 | 5264C > G | 5264C > G | S > X | 1755 | 29 |
| NF-050 | 5294C > A | 5215del90 (5294C > A???) | S > X | 1765 | 29 |
| NF-037 | 5546G > A | 5205del341; 5205del544 | R > Q | 1849 | 29 |
| NF-038 | 5546G > A | 5205del341; 5205del544 | R > Q | 1849 | 29 |
| NF-047 | 5567delT | 5567delT | frameshift | 1856 | 30 |
| NF-031 | 5798delC | 5798delC | frameshift | 1933 | 31 |
| NF-060 | 5839C > T | 5839C > T | R > X | 1947 | 31 |
| NF-043 | 5896C > T | 5896C > T | Q > X | 1966 | 31 |
| NF-015 | 6577delGAGgta | 6364del215 | frameshift | 2193 | 34 |
| NF-040 | 6709C > T | 6709C > T | R > X | 2237 | 36 |
| NF-008 | 6789-6792delTTAC | 6789-6792delTTAC | frameshift | 2263 | 37 |
| NF-019 | 6792C > A | 6756del102 | Y > X | 2264 | 37 |
| NF-003 | 6792C > G | 6756del102 | Y > X | 2264 | 37 |
| NF-032 | 6858G > C | 6756del102 | K > N | 2286 | 37 |
| NF-013 | 7096-7101del6 | 7096-7101del6 | delNF | 2366 | 39 |
| NF-022 | 7096-7101del6 | 7096-7101del6 | delNF | 2366 | 39 |
| NF-005 | IVS39 − 12T > A | 7126del132; | NA | NA | IVS39 |
| | | 7127-10ins10 | | | |
| NF-002 | 7201A > T | 7201A > T | K > X | 2401 | 40 |
| NF-055 | 7285C > T | 7285C > T | R > X | 2429 | 41 |
| NF-061 | 7486C > T | 7486C > T | R > X | 2496 | 42 |
| NF-020 | 7884-7885delGT | 7884-7885delGT | frameshift | 2628 | 45 |
| NF-007 | 8016delA | 8016delA | frameshift | 2672 | 46 |

| | | PTT | | | |
|---|---|---|---|---|---|
| Patient | Type/effect | (frag)² | CG³ | S/F⁴ | Previously described |
| NF-004 | nonsense | +(F1) | no | F | Osborn et al., 1999 |
| NF-023 | missense | −(HA) | no | F | Novel |
| NF-036 | missense | −(HA) | no | F | Novel |
| NF-056 | nonsense | +(F1) | yes | S | Fahsold et al., in press |
| NF-029 | frameshift | +(F1) | NA | F | Novel |
| NF-027 | nonsense: truncation due to stopcodon; | +(F1) | yes | S | Hoffmeyer et al., 1998 |
| | splice: intact 3' and 5'ss, no cryptic or | | | | |
| | novel ss, IF skip E7 | | | | |
| NF-064 | identical as NF-027 | +(F1) | yes | F | Hoffmeyer et al., 1998 |
| NF-024 | frameshift | +(F1) | NA | F | Novel |
| NF-048 | nonsense | +(F1) | no | S | Novel |
| NF-042 | nonsense | +(F1) | yes | S | Heim et al., 1995 |
| NF-057 | nonsense | +(F1) | yes | S | Heim et al., 1995 |
| NF-030 | nonsense | +(F1) | yes | S | Fahsold et al., in press |
| NF-026 | nonsense | +(F1) | yes | S | Fahsold et al., in press |
| NF-017 | splice: intact WT 5'ss, creation novel | +(F1) | no | F | Messiaen et al., 1999 |
| | 5'ss, skip last 62 nt E10b forms | | | | |
| | immediate stopcodon | | | | |
| NF-012 | identical as NF-017 | +(F1) | no | S | Messiaen et al., 1999 |
| NF-016 | nonsense | +(F1) | NA | F | Novel |
| NF-033 | nonsense | +(F1) | no | S | Novel |
| NF-052 | nonsense | +(F1) | no | F | Novel |
| NF-045 | splice: inactive 5'ss, IF skip E11 + 12a | +(F2) | no | S | Abernathy et al., 1997 |
| NF-034 | frameshift | +(F2) | NAo | S | Heim et al., 1995 |
| NF-035 | frameshift | +(F2) | NA | S | Heim et al., 1995 |
| NF-010 | missense | −(HA) | no | F | Messiaen et al., 1998 |
| NF-051 | splice: inact 5'ss, activ cryptic 5'ss skip | +(F2) | NA | S | Novel |
| | last 233 nt E16 | | | | |
| NF-028 | splice: inactiv 3'ss, OOF skip E17 | +(F2) | NA | F | Novel |
| NF-014 | nonsense | +(F2) | no | F | Novel |
| NF-011 | nonsense | +(F2) | no | F | Novel |
| NF-001 | amino acid deletion | −(HA) | NA | F | Shen et al., 1993 |
| NF-053 | frameshift | +(F2) | NA | S | Novel |
| NF-063 | splice: intact WT 5'ss, creation novel | +(F2) | no | F | Novel |
| | 5'ss, skip last 40 nt E19b | | | | |
| NF-025 | splice: inactiv 3'ss, OOF skip E20 | +(F2) | no | F | Novel |
| NF-039 | nonsense | +(F2) | no | S | Novel |
| NF-046 | nonsense | +(F2) | no | S | Novel |
| NF-059 | nonsense | +(F3) | yes | S | Heim et al., 1995 |
| NF-041 | nonsense | +(F3) | yes | S | Upadhyaya et al., 1997 |
| NF-054 | nonsense | +(F3) | yes | S | Upadhyaya et al., 1997 |
| NF-044 | splice: inactiv 3'ss, activ 2 cryptic 3'ss | +(F3) | no | S | Novel |
| | sites leading to 2 different OOF | | | | |
| | insertions | | | | |
| NF-021 | nonsense | +(F3) | yes | S | Side et al., 1995 |
| NF-006 | nonsense | +(F3) | yes | S | Side et al., 1995 |
| NF-058 | splice: inactiv 3'ss, activ cryptic 3'ss, | +(F3) | no | F | Novel |
| | OOF skip E28; OOF skip first 293 nt | | | | |
| | E28 | | | | |
| NF-049 | frameshift | +(F3) | NA | F | Novel |
| NF-009 | nonsense | +(F4) | no | S | Novel |
| NF-050 | splice: intact 3'ss, creation novel 3'ss, IF | +(F4) | yes | F | Novel |
| | skip first 90 nt E29 | | | | |
| NF-037 | splice: inact 5'ss, OOF skip E29 and | +(F4) | yes | F | Ars et al., 2000 |
| | E29 + 30 | | | | |
| NF-038 | identical as NF-037 | +(F4) | yes | F | Ars et al., 2000 |
| NF-047 | frameshift | +(F4) | NA | S | Novel |
| NF-031 | frameshift | +(F4) | NA | S | Novel |
| NF-060 | nonsense | +(F4) | yes | F | Cawthon et al., 1990 |
| NF-043 | nonsense | +(F4) | no | F | novel |
| NF-015 | splice: inactiv 5'ss, OOF skip E34 | +(F4) | NA | S | novel |
| NF-040 | nonsense | +(F4) | yes | S | Fahsold et al., in press |
| NF-008 | frameshift | +(F4) | NA | S | Robinson et al., 1995 |
| NF-019 | splice: intact 3' and 5'ss, no cryptic or | +(F4) | no | F | Messiaen et al., 1997 |
| | novel ss, IF skip E37 | | | | |
| NF-003 | identical as NF-019 | +(F4) | no | F | Messiaen etal, 1997 |
| NF-032 | splice: inactiv 5'ss, IF skip E37 | +(F4) | no | F | novel |
| NF-013 | amino acid deletion | −(HA) | NA | S | Abernathy et al., 1994 |
| NF-022 | amino acid deletion | −(HA) | NA | F | Abernathy et al., 1994 |
| NF-005 | splice: inactiv 3'ss, IF skip E40; activ | +(F5) | no | F | novel |
| | cryptic 3'ss, OOF ins last 10 nt IVS39 | | | | |
| NF-002 | nonsense | +(F5) | no | S | novel |
| NF-055 | nonsense | +(F5) | yes | S | Fahsold et al., in press |
| NF-061 | nonsense | +(F5) | yes | S | Purandare et al., 1994 |
| NF-020 | frameshift | +(F5) | NA | S | novel |
| NF-007 | frameshift | +(F5) | NA | S | novel |

¹Numbering based on NF1 cDNA (Genbank M82814).
²+ denotes that a truncated peptide was seen; − denotes that no truncated peptide was seen but mutation was identified by heteroduplex analysis (HA); (F1 to F5) denotes that the truncated peptide was seen after in vitro translation of RT-PCR fragments F1 to F5 using primers for amplifying NF1 cDNA as described by Heim et al (1995). F1 spans cDNA nt 1-1868, F2 1468-3583, F3 3217-5256, F4 4998-6987 and F5 6574-404.
³For all 44 transitions is mentioned whether they change a G > A or C > T at a CpG dinucleotide.
⁴Sporadic (S) or familial (F) patient.
Abbreviations: E exon/IVS intervening sequence or intron; IF, inframe; OOF, out of frame; ss splice site; nt nucleotide; skip skipping; inactiv. inactivation; activ. activation; NA not applicable

TABLE 6
Comparison of the sensitivity of detecting NF1 mutations by direct
cDNA cycle sequencing starting from Pmin and Pplus EBV cultures as
measured by the ratio between mutant/wild-type peak height on
sequencing chromatograms.

| | | | | With |
|---|---|---|---|---|
| | | | Without | puromycin |
| | | | puromycin | Ratio |
| | | Genomic | ratio | mutant/ |
| Patient | Exon | Mutation | mutant/wild-type | wild-type |
| NF-056 | 4b | R192X | 0.51 | 0.80 |
| NF-064 | 7 | R304X | 0.34 | 1.00 |
| NF-057 | 10a | R440X | 0.60 | 1.00 |
| NF-012 | 10b | Y489C | 0.60 | 1.00 |
| NF-016 | 10b | 1466insC | 0.65 | 1.00 |
| NF-033 | 10c | E524X | 0.35 | 1.00 |
| NF-035 | 13 | 2033insC | 0.35 | 0.76 |
| NF-051 | IVS16 | 2850 + 2deltaaagt | 0.84 | 1.00 |
| NF-063 | 19b | V1093M | 0.58 | 0.92 |
| NF-039 | 20 | E1123X | 0.55 | 1.00 |
| NF-046 | 21 | Q1174X | 0.32 | 0.72 |
| NF-002 | 40 | K2401X | 0.28 | 0.75 |
| NF-020 | 42 | 7884delGT | 0.29 | 0.91 |

TABLE 7
Consensus values (CV) and Splice Site Scores (SSS) of splice sites (ss) involved
in splicing mutations in the NF1 gene

| | Consensus Values | | | | | | |
|---|---|---|---|---|---|---|---|
| | according to Shapiro | | | Splice Site Scores | | | |
| | and Senepathy | | | according to NNPSS | | | |
| Splice site | CV | CV | CVc | SSS- | SSS- | SSS- | |
| (Mutation) | N | M | ry | N | M | cry | Comment |

Mutations at 5'ss

| | | | | | | | | |
|---|---|---|---|---|---|---|---|---|
| 6577delGAGgta | TG(delGAGgta)tagaag | 0.70 | 0.33 | NA | 0.41 | / | NA | skipping E34 |
| IVS12a + 1G > T | AG(g > t)taagc | 0.94 | 0.76 | NA | 1.00 | / | NA | skipping E11 + E12a |
| IVS16 + 2delaaagtg | AGgt(delaaagtg)ttct | 0.81 | 0.64 | 0.82 | 0.92 | 0.12 | 0.95 | activ. cryptic 5'ss 233 nt |
| | | | | | | | | upst. |
| 5546G > A | C(G > A)gtaggt | 0.81 | 0.69 | NA | 0.97 | 0.10 | NA | skipping E29/E29 + E30 |
| (R1849Q) | | | | | | | | |
| 6858G > C | A(G > C)gtaatt | 0.86 | 0.72 | NA | 0.99 | 0.73 | NA | skipping E37 |
| (K2286N) | | | | | | | | |

Mutations at 3'ss

| | | | | | | | | |
|---|---|---|---|---|---|---|---|---|
| IVS26 − 2A > T | tttgctgtatct(a > t)gG | 0.82 | 0.66 | IVS2 | 0.49 | / | IVS2 | activ. cryptic 3'ss 14 nt |
| | | | | 6- | | | 6- | upst. and 17 nt upst. |
| | | | | 14:0.84 | | | 14:0.92 | |
| | | | | IVS2 | | | IVS2 | |
| | | | | 6- | | | 6-17:/ | |
| | | | | 17:0.81 | | | | |
| IVS27b − 2A > T | gtcattttcctt(a > t)gG | 0.84 | 0.68 | 0.84 | 1.00 | / | 0.84 | skipping E28 and activ. |
| | | | | | | | | cryptic 3'ss 293 nt downst |
| IVS16-6delcttt | tatttgttcttt(delcttt)agG | 0.89 | 0.89 | NA | 0.99 | 0.97 | NA | skipping E17 |
| IVS19bC > G | tttttatttct(c > g)agA | 0.96 | 0.84 | NA | 0.96 | 0.04 | NA | skipping E20 |
| IVS39 − 12T > A | tt(t > a)gttttttgtagG | 0.90 | 0.89 | 0.79 | 1.00 | 0.99 | 0.92 | skipping E40 and use of |
| | | | | | | | | novel created 5'ss 10 nt |
| | | | | | | | | downst. |

| | | | | | | | |
|---|---|---|---|---|---|---|---|
| Splice site | CV | CV | CVn | SSS- | SSS- | SSS- | Comment |
| (Mutation) | N | M | ov | N | M | nov | |

Creation of novel 5'ss

| | | | | | | | | |
|---|---|---|---|---|---|---|---|---|
| Y489C | CT(a > g)taagt | 0.79 | NA | 0.79 | 0.86 | NA | 0.97 | novel 5'ss 62 nt upst. |
| V1093M | TGgt(g > a)tgg | 0.73 | NA | 0.76 | 0.13 | NA | 0.27 | novel 5'ss 40 nt upst. |

Creation of novel 3'ss

| | | | | | | | | |
|---|---|---|---|---|---|---|---|---|
| S1765X | aatgacatttattatgctt(c > a)gGA | 0.90 | NA | 0.83 | 0.81 | NA | 0.18 | novel 3'ss 90 nt downst. |

CVN and CVM, consensus value normal and mutant;
CVcry, consensus value of activiated cryptic splice site;
CVnov, consensus value of novel created splice site;
ss, splice site;
E, exon;
upst., upstream;
downst., downstream;
NA, not applicable;
/, no SSS can be obtained by SSPNN not even at the lowest threshold; capital letters denote nucleotides within exons and small letters within introns.

What is claimed is:

1. Method for mutation analysis of the NF1 gene of a patient comprising the steps of:
a) isolating peripheral blood lymphocytes of said patient;
b) establishing an EBV transformed B-lymphoblastoid cell line with said peripheral blood lymphocytes of said patient, or short-term culturing of the blood lymphocytes by phytohaemaglutinin (PHA) stimulation
c) treatment of the EBV transformed B-lymphoblastoid cell line or short-term cultures with a protein synthesis inhibitor with a protein synthesis inhibitor,
d) immediate extraction of RNA of cultures of said EBV transformed B-lymphoblastoid cell line,
e) amplifying said RNA using suitable primers, and,
f) obtaining peptide fragments by means of in vitro transcription/translation of said amplified fragments of step e).

2. Method according to any claim 1 wherein said protein synthesis inhibitor is puromycin, cycloheximide, actinomycinD or possible analogues.

3. Method according to any claims 1 to 2 wherein RNA is extracted within a time period of less than 48 hours at room temperature preferentially immediately after the protein synthesis inhibitor treatment of EBV transformed B-lymphoblastoid cell lines.

4. Method according to any of claims 1 to 3 wherein said amplification step is a polymerase chain reaction.

5. Method according to any of claim 1 to 4 wherein said RNA extracted in step d) as defined in claim 1 is total RNA.

6. Method according to any of claims 1 to 5 wherein step f) as defined in claim 1 is followed by a separation of said peptide fragments.

7. Method according to claim 6 wherein in case a truncated peptide is observed by means of protein separation, the amplified cDNA fragment obtained in step e) of claim 1 is analyzed by means of suitable primers allowing the characterization of some or all exons present in said cDNA fragment.

8. Method according to claim 7 wherein said analysis is performed by means of cycle sequencing of the suitable fragment by means of suitable primers.

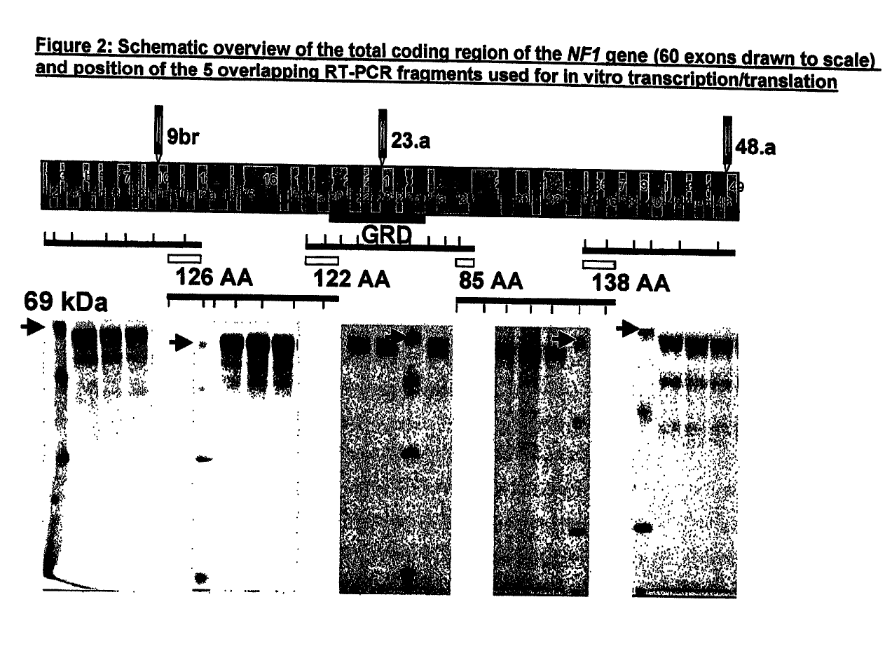

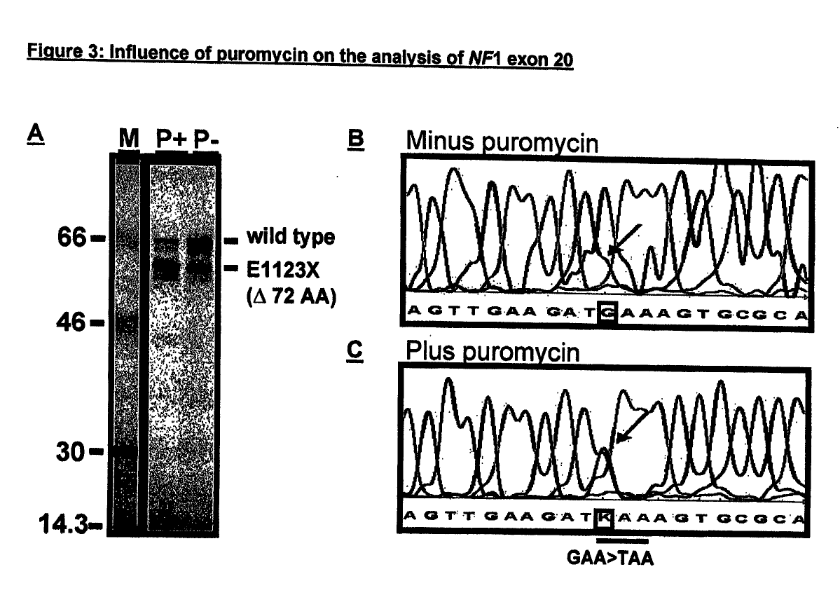

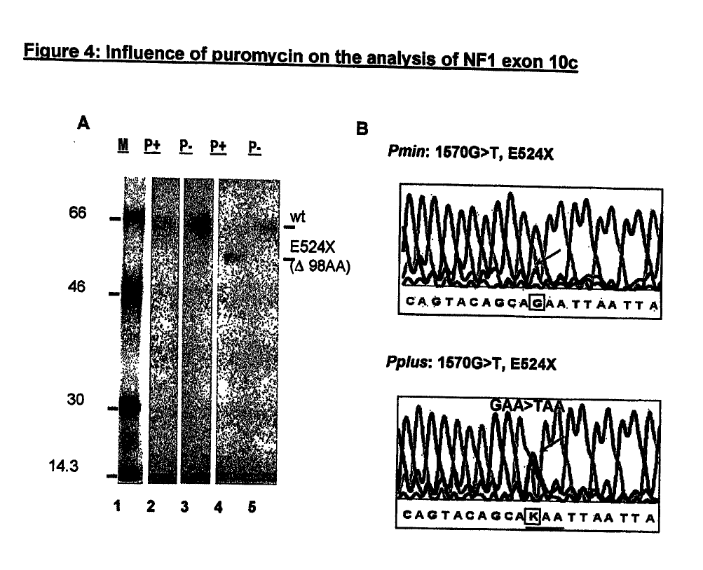

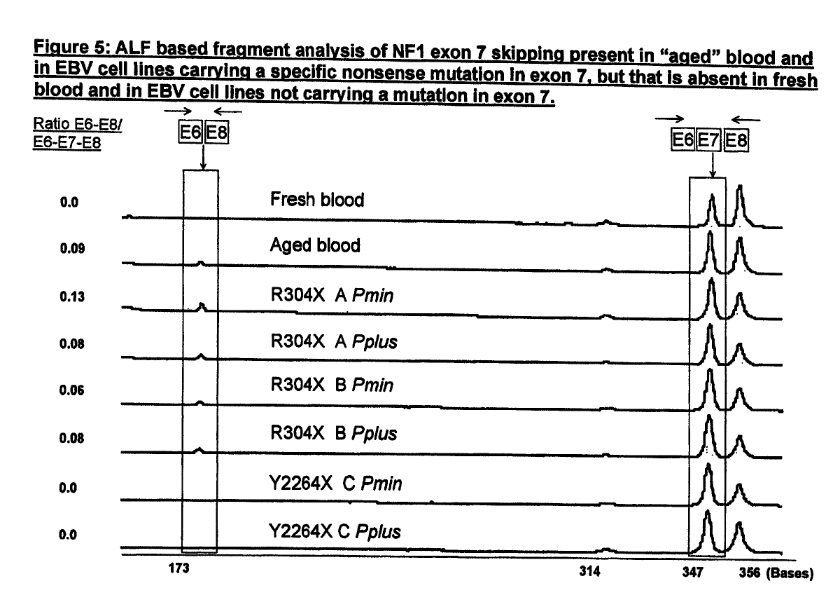

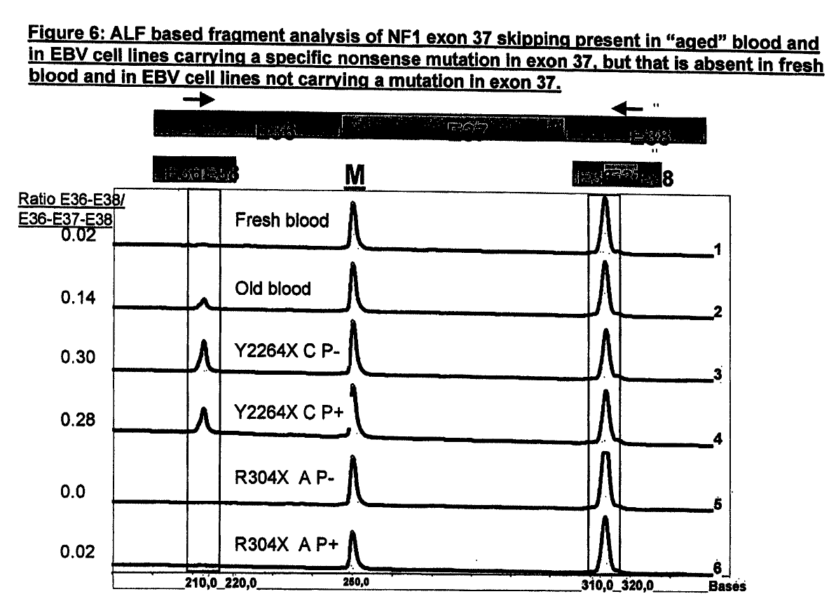

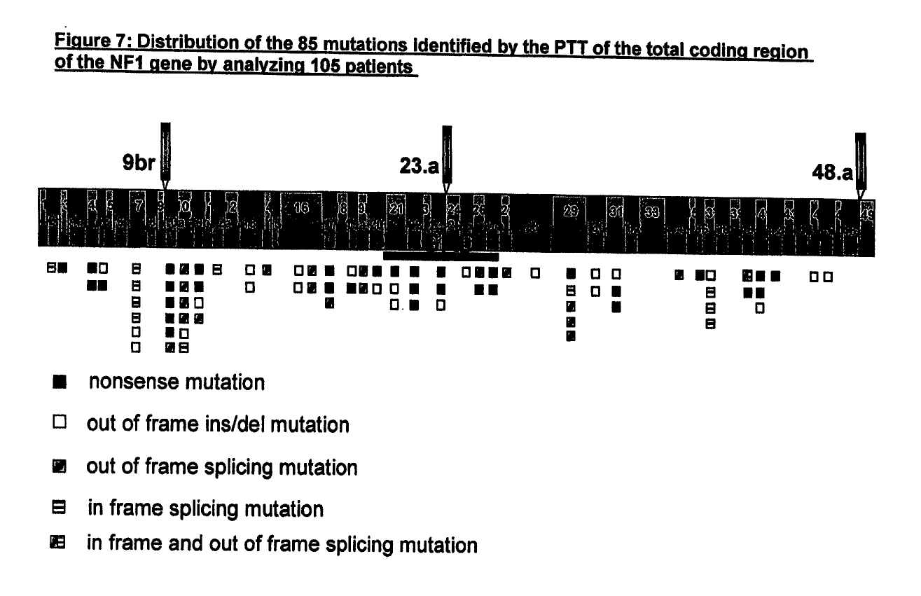

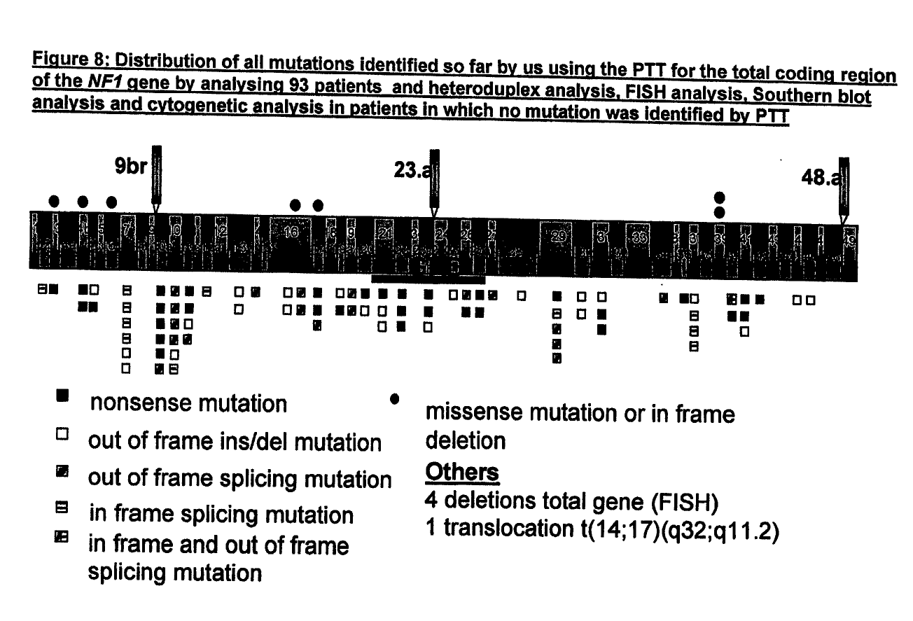

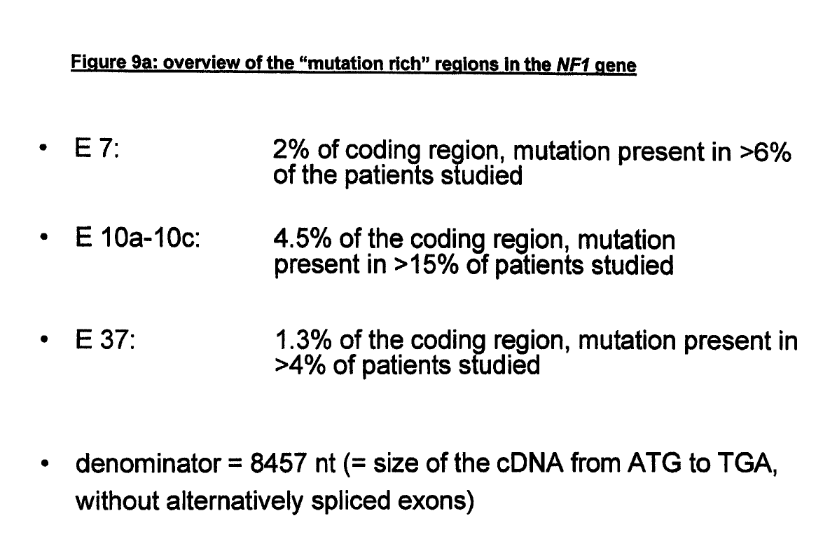

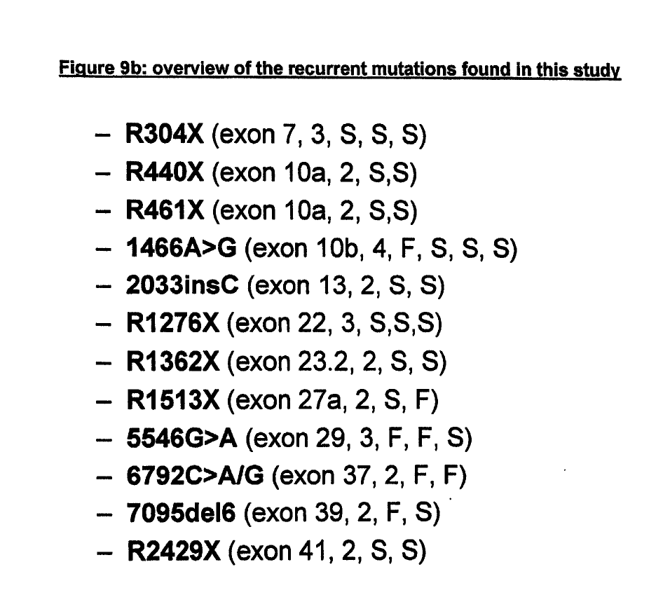

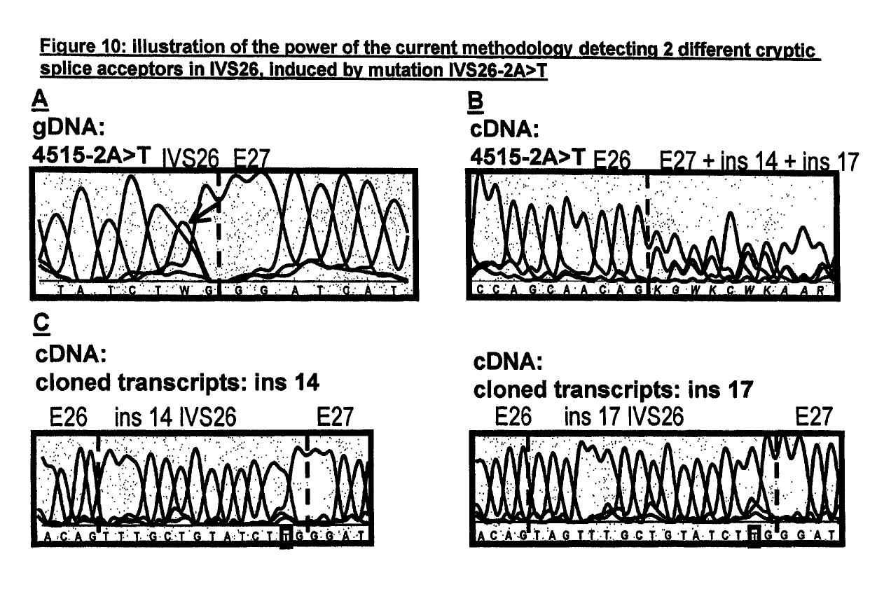

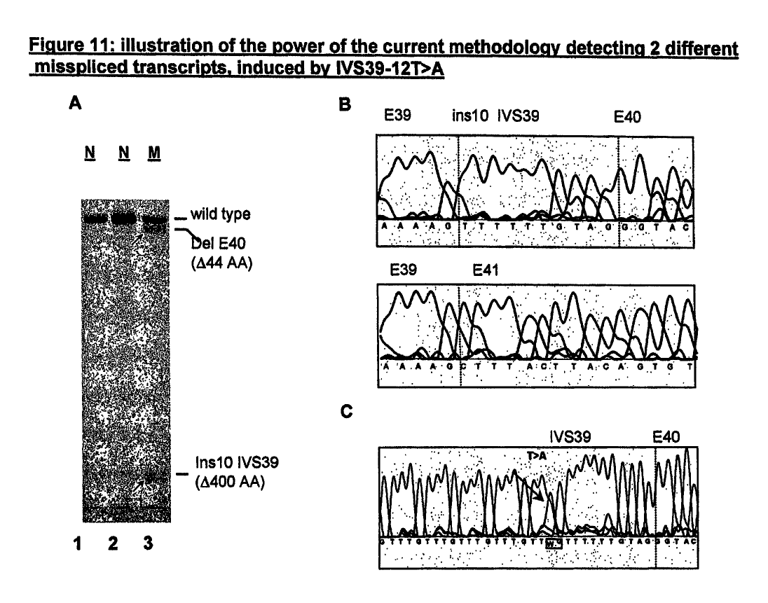

9. Method according to any of the claims 1 to 8 wherein said primers of step e) are represented on FIG. 2 or in any of the tables.

10. Method according to any of the claims 1 to 9 for mutation analysis of the NF1 gene of a patient involving the detection of a frame shift, missense or silent mutation.

11. Method according to any of the claims 1 to 9 wherein said primers are located in exon 4b, 7, 10a-10c, 13, 22, 23.2, 27a, 29, 37 or 39.

12. Method according to any of the claims 1 to 11 wherein said primers are labeled.

13. Method according to any of the claims 1 to 11 wherein said cDNA fragments are further analyzed by means of ALF or another (semi)-automated sequencing method.

14. Method according to any of claims 1 to 13 wherein in addition also DNA is extracted from said peripheral blood lymphocytes of said patient for further analysis of the genomic mutations present in the DNA of said patient.

15. Method according to claim 14 wherein said analysis is performed by means of heteroduplex analysis and/or single stranded conformation polymorphism analysis and/or conformation sensitive gelelectrophoresis (to detect aberrant migrating PCR fragments which are then further analyzed by cycle sequencing) or immediate cycle sequencing.

16. Method for mutation analysis of the NF1 gene of a patient involving the detection of a mutation in exon 7, 10a-10b-10c, 13, 23.2, 27a, 29 or 39.

17. Method for detecting at least one of the following specific mutations of the NF1 gene: K33K (99del105), C93Y (278G>A), C187Y (560G>A), R192X (574C>T), 603-604insT (idem), Q209X (625C>T), 819-821delCCT (idem), 889-454del474nt (888del174), 987-988insA (idem), 1261-19G>A (1260insTTTGTTTTTCTCTAGTC), W425X (1275G>A), R461X (1381C>T), Y489C (1465del62), 1466insC (idem), 1527+5G>A (1392del135), E524X (1570G>T), 1605insA (idem), S536X (1607C>A), 1642-3C>G (1641del80), 2305insT (idem), 2585insA (idem), 2836insT (idem), 2850+2del6 (2617del233), 2851-6del4 (2850del140), Q959X (2875C>T), Q963X (2887C>T), 2990+3A>C (2850del140), Y1044X (3132C>A), 3193delC (idem), V1093M (3277G>A/ 3274 del40), 3108-3C>G (3314del182), E1123X (3367G>T), 3457delCTCA (idem), Q1174X (3520C>T), 3704delA (idem), 3708+1G>C (3496del212), 4026delG (idem), 4299delC (idem), Q1494X (4480C>T), 4515-2A>T (4515-14ins14/4515-17ins17), 4773-2A>T (4772del433/4772del293), 5033delG (idem), 5117delT (idem), R1849 (502del341/5205del544), S1755X (5264C>G), S1765X (5215del90/5294C>A), 5567delT (idem), 5798delC (idem), Q1966X (5896C>T), 6577delGAGgta (6364del215), R2237X (6709C>T), 6858G>C (6756del102), 7127-12T>A (7126del132/7127-10ins10), 7268delCA (idem), K2401X (7201A>T), R2429X (7285C>T), 7884-7885delGT (idem), 8016delA (idem); with said letters and numbering referring to the mutation at the amino acid level which is mentioned first and the effect of said mutation at the mRNA level being mentioned between parentheses; or the genomic mutation t(14;17)(q32;q11.2) interrupting the NF1 gene.

18. Diagnostic kit comprising primers specifically amplifying the mutation region or positions as defined in claim 16 or 17.

19. Diagnostic kit comprising a probe specifically detecting a mutation region or position as defined in claim 16 or 17.

20. Method for identifying a compound correcting the defective structure of the mutated NF1 protein, coded by a mutated DNA as described in claim 17.

21. Model systems comprising an NF1 gene mutation as defined by claim 17 which can be used to screen for a therapeutic agent.


SEQUENCE LISTING (264 sequences)

SEQ ID NO: 1 - DNA, 17 bp, Homo sapiens
tttgtttttctctagtc

SEQ ID NO: 2 - DNA, 18 bp, Homo sapiens
ttgacttggtggtggttt

SEQ ID NO: 3 - DNA, 20 bp, Homo sapiens
ttgagaatggcttacttgga

SEQ ID NO: 4 - DNA, 31 bp, Homo sapiens
gtttgtttgtttgtttgttagttttttgtag

SEQ ID NO: 5 - DNA, 18 bp, Homo sapiens
cttcggaattctgcctct

SEQ ID NO: 6 - DNA, 18 bp, Homo sapiens
ctgatatggctgaatgtg

SEQ ID NO: 7 - DNA, 18 bp, Homo sapiens
gcctgtgtcaaactgtgt

SEQ ID NO: 8 - DNA, 18 bp, Homo sapiens
cacacccagcaatacgaa

SEQ ID NO: 9 - DNA, 15 bp, Homo sapiens
gctttgtgtaagtat

SEQ ID NO: 10 - DNA, 15 bp, Homo sapiens
agaagctgtaagtat

SEQ ID NO: 11 - DNA, 19 bp, Homo sapiens
ttgacttggtggatggttt

SEQ ID NO: 12 - DNA, 20 bp, Homo sapiens
gggcagataaagcagataat

SEQ ID NO: 13 - DNA, 18 bp, Homo sapiens
ccggattgccataaatac

SEQ ID NO: 14 - DNA, 26 bp, Homo sapiens
gtttgttagttttttgtagggtacag

SEQ ID NO: 15 - DNA, 15 bp, Homo sapiens
ttgtttttctctagc

SEQ ID NO: 16 - DNA, 20 bp, Homo sapiens
atggccgcgcacaggccggt

SEQ ID NO: 17 - DNA, 18 bp, Homo sapiens
acaggacagcagaacaca

SEQ ID NO: 18 - DNA, 18 bp, Homo sapiens
cttcggaattctgcctct

SEQ ID NO: 19 - DNA, 18 bp, Homo sapiens
ctgatatggctgaatgtg

SEQ ID NO: 20 - DNA, 18 bp, Homo sapiens
gcctgtgtcaaactgtgt

SEQ ID NO: 21 - DNA, 18 bp, Homo sapiens
cacacccagcaatacgaa

SEQ ID NO: 22 - DNA, 18 bp, Homo sapiens
atcctgatgctcctgtag

SEQ ID NO: 23 - DNA, 18 bp, Homo sapiens
tttttacggggtaggatg

SEQ ID NO: 24 - DNA, 18 bp, Homo sapiens
atttgccgacaagcccag

SEQ ID NO: 25 - DNA, 18 bp, Homo sapiens
actgcaggaaacactgag

SEQ ID NO: 26 - DNA, 18 bp, Homo sapiens
ggctgttgtccttaatgg

SEQ ID NO: 27 - DNA, 17 bp, Homo sapiens
tgcttgggaatatggtc

SEQ ID NO: 28 - DNA, 20 bp, Homo sapiens
gtttcacttctagctggtct

SEQ ID NO: 29 - DNA, 20 bp, Homo sapiens
caaacaggtggcaggaaacg

SEQ ID NO: 30 - DNA, 18 bp, Homo sapiens
ccttcaacaaggcacaga

SEQ ID NO: 31 - DNA, 18 bp, Homo sapiens
actctaccaactgctctg

SEQ ID NO: 32 - DNA, 18 bp, Homo sapiens
gaacctccttcagatgac

SEQ ID NO: 33 - DNA, 18 bp, Homo sapiens
agaagaacatatgcggcc

SEQ ID NO: 34 - DNA, 20 bp, Homo sapiens
gcacattgccgtcacttatg

SEQ ID NO: 35 - DNA, 18 bp, Homo sapiens
ctaggcatcaggtacatg

SEQ ID NO: 36 - DNA, 18 bp, Homo sapiens
gtctccgcagtctatatc

SEQ ID NO: 37 - DNA, 20 bp, Homo sapiens
cctgggaaactggctgagca

SEQ ID NO: 38 - DNA, 18 bp, Homo sapiens
ggagtgtgaagccattgt

SEQ ID NO: 39 - DNA, 18 bp, Homo sapiens
tagtaagacgctggcagc

SEQ ID NO: 40 - DNA, 18 bp, Homo sapiens
ccttgggcagattacaga

SEQ ID NO: 41 - DNA, 18 bp, Homo sapiens
gatgctgtccttcaacaa

SEQ ID NO: 42 - DNA, 18 bp, Homo sapiens
gaaacagtcacagaagct

SEQ ID NO: 43 - DNA, 20 bp, Homo sapiens
ggaggcatgcatgagagata

SEQ ID NO: 44 - DNA, 19 bp, Homo sapiens
cagccacttcttaataagg

SEQ ID NO: 45 - DNA, 19 bp, Homo sapiens
gcatccttcacctgctatt

SEQ ID NO: 46 - DNA, 18 bp, Homo sapiens
catggtgacccttcctat

SEQ ID NO: 47 - DNA, 18 bp, Homo sapiens
atgttctcttggatgaag

SEQ ID NO: 48 - DNA, 18 bp, Homo sapiens
gctgagcttattgttaag

SEQ ID NO: 49 - DNA, 20 bp, Homo sapiens
cccagcctccttgccaacgc

SEQ ID NO: 50 - DNA, 20 bp, Homo sapiens
gacccattccaccggcctgt

SEQ ID NO: 51 - DNA, 18 bp, Homo sapiens
tttcaatggcaagtaagt

SEQ ID NO: 52 - DNA, 19 bp, Homo sapiens
gttatatccaaagtccaca

SEQ ID NO: 53 - DNA, 23 bp, Homo sapiens
tttaaggataaactgtttacgtg

SEQ ID NO: 54 - DNA, 23 bp, Homo sapiens
tttcacttttcagatgtgtgttg

SEQ ID NO: 55 - DNA, 20 bp, Homo sapiens
tggtccacatctgtactttg

SEQ ID NO: 56 - DNA, 19 bp, Homo sapiens
ttaaatctaggtggtgtgt

SEQ ID NO: 57 - DNA, 18 bp, Homo sapiens
aaactcatttctctggag

SEQ ID NO: 58 - DNA, 21 bp, Homo sapiens
ggcttcctgaagtgctgggat

SEQ ID NO: 59 - DNA, 24 bp, Homo sapiens
ccagtttggtgttctagttcagca

SEQ ID NO: 60 - DNA, 21 bp, Homo sapiens
gtgagataccacacctgtccc

SEQ ID NO: 61 - DNA, 22 bp, Homo sapiens
tttcctagcagacaactatcga

SEQ ID NO: 62 - DNA, 26 bp, Homo sapiens
catcaaaaaaaaaattttaataccag

SEQ ID NO: 63 - DNA, 23 bp, Homo sapiens
catgtggttctttatttataggc

SEQ ID NO: 64 - DNA, 21 bp, Homo sapiens
tcaatcgtatccttaccagcc

SEQ ID NO: 65 - DNA, 26 bp, Homo sapiens
catgtttatcttttaaaaatgttgcc

SEQ ID NO: 66 - DNA, 24 bp, Homo sapiens
ataatggaaataattttgccctcc

SEQ ID NO: 67 - DNA, 24 bp, Homo sapiens
tgctataatattagctacatctgg

SEQ ID NO: 68 - DNA, 23 bp, Homo sapiens
cctatgaacttatcaacgaagag

SEQ ID NO: 69 - DNA, 24 bp, Homo sapiens
tgctataatattagctacatctgg

SEQ ID NO: 70 - DNA, 24 bp, Homo sapiens
ctagtctttctgtttataaaggat

SEQ ID NO: 71 - DNA, 20 bp, Homo sapiens
tccgctgtggctcagaacac

SEQ ID NO: 72 - DNA, 21 bp, Homo sapiens
agtagaagaggatgcacagcc

SEQ ID NO: 73 - DNA, 24 bp, Homo sapiens
tttgacctcatttgtattactgag

SEQ ID NO: 74 - DNA, 23 bp, Homo sapiens
agaaccttttgaaaccaagagtg

SEQ ID NO: 75 - DNA, 24 bp, Homo sapiens
acgtaattttgtactttttcttcc

SEQ ID NO: 76 - DNA, 22 bp, Homo sapiens
caatagaaaggaggtgagattc

SEQ ID NO: 77 - DNA, 22 bp, Homo sapiens
cttggtaccctttagcagtcac

SEQ ID NO: 78 - DNA, 18 bp, Homo sapiens
ccttctttctccatggag

SEQ ID NO: 79 - DNA, 22 bp, Homo sapiens
gtactccagtgttatgtttacc

SEQ ID NO: 80 - DNA, 28 bp, Homo sapiens
taaagttgaaatttaaaaattaaagtac

SEQ ID NO: 81 - DNA, 24 bp, Homo sapiens
acttgtattcattatgggagaatg

SEQ ID NO: 82 - DNA, 24 bp, Homo sapiens
agtaatctctcaccattaccattc

SEQ ID NO: 83 - DNA, 23 bp, Homo sapiens
tttctagtgaatctccttcaagt

SEQ ID NO: 84 - DNA, 25 bp, Homo sapiens
atgaaatttaccaaatttcattcag

SEQ ID NO: 85 - DNA, 20 bp, Homo sapiens
gagttattgtatgcggagac

SEQ ID NO: 86 - DNA, 19 bp, Homo sapiens
ttgaatttcccctgtaaac

SEQ ID NO: 87 - DNA, 23 bp, Homo sapiens
cacagtttattgcattgttagat

SEQ ID NO: 88 - DNA, 21 bp, Homo sapiens
tccttttgggtggagcttatc

SEQ ID NO: 89 - DNA, 23 bp, Homo sapiens
tatacttgtaatatgcacgtatc

SEQ ID NO: 90 - DNA, 23 bp, Homo sapiens
tgtgatcaggaatagcttttgaa

SEQ ID NO: 91 - DNA, 24 bp, Homo sapiens
ttaacagataaaagtcaactttac

SEQ ID NO: 92 - DNA, 24 bp, Homo sapiens
tggataaagcataatttgtcaagt

SEQ ID NO: 93 - DNA, 23 bp, Homo sapiens
tagagaaaggtgaaaaataagag

SEQ ID NO: 94 - DNA, 20 bp, Homo sapiens
ccagtcagtgaacgtaaggg

SEQ ID NO: 95 - DNA, 22 bp, Homo sapiens
ctctgtgtgtttagatcagtca

SEQ ID NO: 96 - DNA, 24 bp, Homo sapiens
tttatcaattactaccagtatcag

SEQ ID NO: 97 - DNA, 24 bp, Homo sapiens
tagtaaggtagccagaagttgtgt

SEQ ID NO: 98 - DNA, 23 bp, Homo sapiens
atttacaaaaccctacattgctc

SEQ ID NO: 99 - DNA, 22 bp, Homo sapiens
tcatgtcacttaggttatctgg

SEQ ID NO: 100 - DNA, 24 bp, Homo sapiens
tgtaattaagtagttataactctc

SEQ ID NO: 101 - DNA, 22 bp, Homo sapiens
tcatgtcacttaggttatctgg

SEQ ID NO: 102 - DNA, 20 bp, Homo sapiens
attaccttctccccatttga

SEQ ID NO: 103 - DNA, 19 bp, Homo sapiens
ggctttatttgctttttgc

SEQ ID NO: 104 - DNA, 19 bp, Homo sapiens
ccaccctggctgattatcg

SEQ ID NO: 105 - DNA, 23 bp, Homo sapiens
taatttttgcttctcttacatgc

SEQ ID NO: 106 - DNA, 26 bp, Homo sapiens
ctatatcaggtaaaatcatgtccaac

SEQ ID NO: 107 - DNA, 21 bp, Homo sapiens
gatttgctatgtgccagggac

SEQ ID NO: 108 - DNA, 19 bp, Homo sapiens
gtcaaacttactcaatgcc

SEQ ID NO: 109 - DNA, 17 bp, Homo sapiens
caaccacttccctacag

SEQ ID NO: 110 - DNA, 19 bp, Homo sapiens
aactggcatgtaagagaag

SEQ ID NO: 111 - DNA, 21 bp, Homo sapiens
tgctactctttagcttcctac

SEQ ID NO: 112 - DNA, 22 bp, Homo sapiens
ccttaaaagaagacaatcagcc

SEQ ID NO: 113 - DNA, 19 bp, Homo sapiens
gattgggtctcaacatttc

SEQ ID NO: 114 - DNA, 23 bp, Homo sapiens
  (21)..(21): N is any nucleotide
aataggctgaagtgaagatantc

SEQ ID NO: 115 - DNA, 24 bp, Homo sapiens
tttgtatcattcattttgtgtgta

SEQ ID NO: 116 - DNA, 24 bp, Homo sapiens
aaaaacacggttctatgtgaaaag

SEQ ID NO: 117 - DNA, 23 bp, Homo sapiens
cttaatgtctgtataagagtctc

SEQ ID NO: 118 - DNA, 25 bp, Homo sapiens
actttagattaataatggtaatctc

SEQ ID NO: 119 - DNA, 24 bp, Homo sapiens
ttgaactctttgttttcatgtctt

SEQ ID NO: 120 - DNA, 24 bp, Homo sapiens
ggaatttaagatagctagattatc

SEQ ID NO: 121 - DNA, 26 bp, Homo sapiens
aatataataattatatttgggaaggt

SEQ ID NO: 122 - DNA, 24 bp, Homo sapiens
gaaaatatttgattcaaacagagc

SEQ ID NO: 123 - DNA, 26 bp, Homo sapiens
aatataataattatatttgggaaggt

SEQ ID NO: 124 - DNA, 21 bp, Homo sapiens
cattttattatagcagatgtc

SEQ ID NO: 125 - DNA, 19 bp, Homo sapiens
acttacacaggaacttcat

SEQ ID NO: 126 - DNA, 22 bp, Homo sapiens
gctttgtctaatgtcaagtcac

SEQ ID NO: 127 - DNA, 23 bp, Homo sapiens
ttaaacggagagtgttcactatc

SEQ ID NO: 128 - DNA, 24 bp, Homo sapiens
gttacaagttaaagaaatgtgtag

SEQ ID NO: 129 - DNA, 24 bp, Homo sapiens
ctaacaagtggcctggtggcaaac

SEQ ID NO: 130 - DNA, 26 bp, Homo sapiens
tttattgtttatccaattatagactt

SEQ ID NO: 131 - DNA, 25 bp, Homo sapiens
tcctgttaagtcaactgggaaaaac

SEQ ID NO: 132 - DNA, 29 bp, Homo sapiens
cactgctaataatctttgtcttttttgtc

SEQ ID NO: 133 - DNA, 27 bp, Homo sapiens
cgtttacaaaacacagactggaactta

SEQ ID NO: 134 - DNA, 23 bp, Homo sapiens
ttccttaggttcaaaactggtca

SEQ ID NO: 135 - DNA, 17 bp, Homo sapiens
tacaatggtgggaactc

SEQ ID NO: 136 - DNA, 21 bp, Homo sapiens
atattaaggtagaggctgttt

SEQ ID NO: 137 - DNA, 19 bp, Homo sapiens
attcttctccacttcaccc

SEQ ID NO: 138 - DNA, 20 bp, Homo sapiens
cccaaatcaaactgaagaga

SEQ ID NO: 139 - DNA, 18 bp, Homo sapiens
tggaactataaggaaaaa

SEQ ID NO: 140 - DNA, 17 bp, Homo sapiens
aaagtcttcactggaaa

SEQ ID NO: 141 - DNA, 25 bp, Homo sapiens
ataattgttgatgtgattttcattg

SEQ ID NO: 142 - DNA, 21 bp, Homo sapiens
aattttgaaccagatgaagag

SEQ ID NO: 143 - DNA, 20 bp, Homo sapiens
ggtagagtgattaaaacatg

SEQ ID NO: 144 - DNA, 20 bp, Homo sapiens
tatgctatagtacagaaggc

SEQ ID NO: 145 - DNA, 24 bp, Homo sapiens
catatctgttttatcatcaggagg

SEQ ID NO: 146 - DNA, 24 bp, Homo sapiens
aagtaaaatggagaaaggaactgg

SEQ ID NO: 147 - DNA, 24 bp, Homo sapiens
caaaatgaaacatggaactttaga

SEQ ID NO: 148 - DNA, 24 bp, Homo sapiens
taagcattaagtacaaatagcaca

SEQ ID NO: 149 - DNA, 20 bp, Homo sapiens
agaagccaaaatgataagaa

SEQ ID NO: 150 - DNA, 18 bp, Homo sapiens
acccaaagacaacaagag

SEQ ID NO: 151 - DNA, 17 bp, Homo sapiens
ggaccagtggacagaac

SEQ ID NO: 152 - DNA, 21 bp, Homo sapiens
atatgctttacaacttgagaa

SEQ ID NO: 153 - DNA, 20 bp, Homo sapiens
tacattaagctagctaccaa

SEQ ID NO: 154 - DNA, 20 bp, Homo sapiens
cgcttgagaacatactatcc

SEQ ID NO: 155 - DNA, 17 bp, Homo sapiens
ccagctaacagtgtctt

SEQ ID NO: 156 - DNA, 21 bp, Homo sapiens
aaggaaatatactcacaataa

SEQ ID NO: 157 - DNA, 19 bp, Homo sapiens
gaacctaatcaaccatctc

SEQ ID NO: 158 - DNA, 20 bp, Homo sapiens
ttgcatttaaagtaagacat

SEQ ID NO: 159 - DNA, 21 bp, Homo sapiens
cctttccttgcagagttgtta

SEQ ID NO: 160 - DNA, 22 bp, Homo sapiens
caccactaaaggactagactgt

SEQ ID NO: 161 - DNA, 25 bp, Homo sapiens
ttcatcctgttttaagtcacacttg

SEQ ID NO: 162 - DNA, 24 bp, Homo sapiens
ttgcctccattagttggaaaattg

SEQ ID NO: 163 - DNA, 25 bp, Homo sapiens
ttcatcctgttttaagtcacacttg

SEQ ID NO: 164 - DNA, 24 bp, Homo sapiens
cttggaaggagcaaacgatggttg

SEQ ID NO: 165 - DNA, 25 bp, Homo sapiens
caaaaactttgctacactgacatgg

SEQ ID NO: 166 - DNA, 24 bp, Homo sapiens
agacactgtagttaatgaacttgc

SEQ ID NO: 167 - DNA, 24 bp, Homo sapiens
catgtactctcccaccttattttc

SEQ ID NO: 168 - DNA, 24 bp, Homo sapiens
agacactgtagttaatgaacttgc

SEQ ID NO: 169 - DNA, 24 bp, Homo sapiens
cacgttaattccctatcttgctgc

SEQ ID NO: 170 - DNA, 24 bp, Homo sapiens
taaaaatttgagggtgggggactc

SEQ ID NO: 171 - DNA, 24 bp, Homo sapiens
catgaataggatacagtcttctac

SEQ ID NO: 172 - DNA, 24 bp, Homo sapiens
cacattactgggtaagcatttaac

SEQ ID NO: 173 - DNA, 24 bp, Homo sapiens
gggaatgtatattatgttttccac

SEQ ID NO: 174 - DNA, 24 bp, Homo sapiens
atgttaggaagttcatcaaccatc

SEQ ID NO: 175 - DNA, 25 bp, Homo sapiens
ctgttacaattaaaagataccttgc

SEQ ID NO: 176 - DNA, 24 bp, Homo sapiens
tgtgtgttcttaaagcaggcatac

SEQ ID NO: 177 - DNA, 24 bp, Homo sapiens
ttttggcttcagatggggatttac

SEQ ID NO: 178 - DNA, 24 bp, Homo sapiens
aagggaattcctaatgttggtgtc

SEQ ID NO: 179 - DNA, 25 bp, Homo sapiens
atctagtatctaattgtatttcacc

SEQ ID NO: 180 - DNA, 21 bp, Homo sapiens
gcagactgagcttacagggac

SEQ ID NO: 181 - DNA, 18 bp, Homo sapiens
tttcaatggcaagtaagt

SEQ ID NO: 182 - DNA, 19 bp, Homo sapiens
gttatatccaaagtccaca

SEQ ID NO: 183 - DNA, 19 bp, Homo sapiens
ttaaatctaggtggtgtgt

SEQ ID NO: 184 - DNA, 18 bp, Homo sapiens
aaactcatttctctggag

SEQ ID NO: 185 - DNA, 20 bp, Homo sapiens
gagttattgtatgcggagac

SEQ ID NO: 186 - DNA, 19 bp, Homo sapiens
ttgaatttcccctgtaaac

SEQ ID NO: 187 - DNA, 20 bp, Homo sapiens
attaccttctccccatttga

SEQ ID NO: 188 - DNA, 19 bp, Homo sapiens
ggctttatttgctttttgc

SEQ ID NO: 189 - DNA, 19 bp, Homo sapiens
gtcaaacttactcaatgcc

SEQ ID NO: 190 - DNA, 17 bp, Homo sapiens
caaccacttccctacag

SEQ ID NO: 191 - DNA, 19 bp, Homo sapiens
gattgggtctcaacatttc

SEQ ID NO: 192 - DNA, 23 bp, Homo sapiens
  (21)..(21): N is any nucleotide
aataggctgaagtgaagatantc

SEQ ID NO: 193 - DNA, 17 bp, Homo sapiens
tacaatggtgggaactc

SEQ ID NO: 194 - DNA, 21 bp, Homo sapiens
atattaaggtagaggctgttt

SEQ ID NO: 195 - DNA, 18 bp, Homo sapiens
tggaactataaggaaaaa

SEQ ID NO: 196 - DNA, 17 bp, Homo sapiens
aaagtcttcactggaaa

SEQ ID NO: 197 - DNA, 20 bp, Homo sapiens
ggtagagtgattaaaacatg

SEQ ID NO: 198 - DNA, 20 bp, Homo sapiens
tatgctatagtacagaaggc

SEQ ID NO: 199 - DNA, 20 bp, Homo sapiens
agaagccaaaatgataagaa

SEQ ID NO: 200 - DNA, 18 bp, Homo sapiens
acccaaagacaacaagag

SEQ ID NO: 201 - DNA, 17 bp, Homo sapiens
ggaccagtggacagaac

SEQ ID NO: 202 - DNA, 21 bp, Homo sapiens
atatgctttacaacttgagaa

SEQ ID NO: 203 - DNA, 20 bp, Homo sapiens
tacattaagctagctaccaa

SEQ ID NO: 204 - DNA, 20 bp, Homo sapiens
cgcttgagaacatactatcc

SEQ ID NO: 205 - DNA, 17 bp, Homo sapiens
ccagctaacagtgtctt

SEQ ID NO: 206 - DNA, 21 bp, Homo sapiens
aaggaaatatactcacaataa

SEQ ID NO: 207 - DNA, 19 bp, Homo sapiens
gaacctaatcaaccatctc

SEQ ID NO: 208 - DNA, 20 bp, Homo sapiens
ttgcatttaaagtaagacat

SEQ ID NO: 209 - DNA, 21 bp, Homo sapiens
cctttccttgcagagttgtta

SEQ ID NO: 210 - DNA, 22 bp, Homo sapiens
caccactaaaggactagactgt

SEQ ID NO: 211 - DNA, 24 bp, Homo sapiens
cacgttaattccctatcttgctgc

SEQ ID NO: 212 - DNA, 24 bp, Homo sapiens
taaaaatttgagggtgggggactc

SEQ ID NO: 213 - DNA, 8 bp, Homo sapiens
tgtagaag

SEQ ID NO: 214 - DNA, 8 bp, Homo sapiens
agttaagc

SEQ ID NO: 215 - DNA, 8 bp, Homo sapiens
aggtttct

SEQ ID NO: 216 - DNA, 8 bp, Homo sapiens
cagtaggt

SEQ ID NO: 217 - DNA, 8 bp, Homo sapiens
acgtaatt

SEQ ID NO: 218 - DNA, 15 bp, Homo sapiens
tttgctgtatcttgg

SEQ ID NO: 219 - DNA, 15 bp, Homo sapiens
gtcattttcctttgg

SEQ ID NO: 220 - DNA, 15 bp, Homo sapiens
tatttgttctttagg

SEQ ID NO: 221 - DNA, 15 bp, Homo sapiens
tttttatttctgaga

SEQ ID NO: 222 - DNA, 15 bp, Homo sapiens
ttagttttttgtagg

SEQ ID NO: 223 - DNA, 8 bp, Homo sapiens
ctgtaagt

SEQ ID NO: 224 - DNA, 8 bp, Homo sapiens
tggtatgg

SEQ ID NO: 225 - DNA, 23 bp, Homo sapiens
aatgacatttattatgcttagga

SEQ ID NO: 226 - DNA, 21 bp, Homo sapiens
agttgaagatgaaagtgcgca

SEQ ID NO: 227 - DNA, 21 bp, Homo sapiens
agttgaagatkaaagtgcgca

SEQ ID NO: 228 - DNA, 20 bp, Homo sapiens
cagtacagcagaattaatta

SEQ ID NO: 229 - DNA, 20 bp, Homo sapiens
cagtacagcakaattaatta

SEQ ID NO: 230 - DNA, 14 bp, Homo sapiens
tatctwgggatcat

SEQ ID NO: 231 - DNA, 20 bp, Homo sapiens
ccagcaacagkgwkcwkaar

SEQ ID NO: 232 - DNA, 22 bp, Homo sapiens
acagtttgctgtatcttgggat

SEQ ID NO: 233 - DNA, 25 bp, Homo sapiens
acagtagtttgctgtatcttgggat

SEQ ID NO: 234 - DNA, 20 bp, Homo sapiens
aaaagttttttgtagggtac

SEQ ID NO: 235 - DNA, 20 bp, Homo sapiens
aaaagctttacttacagtgt

SEQ ID NO: 236 - DNA, 36 bp, Homo sapiens
gtttgtttgtttgtttgttwgttttttgtagggtac

SEQ ID NO: 237 - DNA, 18 bp, Homo sapiens
agaagctrtaagtatctt

SEQ ID NO: 238 - DNA, 18 bp, Homo sapiens
agaagctaatccaagaaa

SEQ ID NO: 239 - DNA, 11 bp, Homo sapiens
agctataagta

SEQ ID NO: 240 - DNA, 15 bp, Homo sapiens
gctttgtgtaagtat

SEQ ID NO: 241 - DNA, 11 bp, Homo sapiens
agctgtaagta

SEQ ID NO: 242 - DNA, 15 bp, Homo sapiens
gctttgtgtaagtat

SEQ ID NO: 243 - DNA, 20 bp, Homo sapiens
ggagatggtrtggaattgat

SEQ ID NO: 244 - DNA, 21 bp, Homo sapiens
aggagatgrtrykkmaywkww

SEQ ID NO: 245 - DNA, 21 bp, Homo sapiens
aggagatggtgtggaattgat

SEQ ID NO: 246 - DNA, 21 bp, Homo sapiens
aggagatgatacttcacatta

SEQ ID NO: 247 - DNA, 12 bp, Homo sapiens
gatggtgtggaa

SEQ ID NO: 248 - DNA, 11 bp, Homo sapiens
cttaagtaaat

SEQ ID NO: 249 - DNA, 12 bp, Homo sapiens
gatggtatggaa

SEQ ID NO: 250 - DNA, 12 bp, Homo sapiens
cttaagtaaatt

SEQ ID NO: 251 - DNA, 20 bp, Homo sapiens
attatgcttmggaaattgaa

SEQ ID NO: 252 - DNA, 19 bp, Homo sapiens
attaaagwwrktkmwrmwr

SEQ ID NO: 253 - DNA, 25 bp, Homo sapiens
ccaccactttccaggttggttctac

SEQ ID NO: 254 - DNA, 20 bp, Homo sapiens
tttattatgcttcggaaatt

SEQ ID NO: 255 - DNA, 25 bp, Homo sapiens
ccaccactttccaggttggttctac

SEQ ID NO: 256 - DNA, 20 bp, Homo sapiens
tttattatgcttaggaaatt

SEQ ID NO: 257 - DNA, 11 bp, Homo sapiens
agtctaygaaa

SEQ ID NO: 258 - DNA, 11 bp, Homo sapiens
agtctacgaaa

SEQ ID NO: 259 - DNA, 11 bp, Homo sapiens
agtctaygaaa

SEQ ID NO: 260 - protein, 19 aa, Homo sapiens
KYTTDEFDQRILYEYLAEA

SEQ ID NO: 261 - protein, 19 aa, Rattus sp.
KYTTDEFDQRILYEYLAEA

SEQ ID NO: 262 - protein, 19 aa, Mus musculus
KYTTDEFDQRILYEYLAEA

SEQ ID NO: 263 - protein, 19 aa, Fugu rubripes
KYSTDDFDQRILYEYLAEA

SEQ ID NO: 264 - protein, 19 aa, Drosophila melanogaster
KYSSDRGETRVLYQYLAEG